(19) United States
(12) Patent Application Publication
(10) Pub. No.: US 2024/0279651 A1
(43) Pub. Date: Aug. 22, 2024

(54) NUCLEIC ACID NANOPARTICLES, PHARMACEUTICAL COMPOSITIONS, AND METHODS OF USE THEREOF

(71) Applicant: IOWA STATE UNIVERSITY RESEARCH FOUNDATION INC., Ames, IA (US)

(72) Inventors: Eric HENDERSON, Ames, IA (US); Chang Yong OH, Ames, IA (US)

(21) Appl. No.: 18/583,301

(22) Filed: Feb. 21, 2024

Related U.S. Application Data
(60) Provisional application No. 63/447,258, filed on Feb. 21, 2023.

(51) Int. Cl. C12N 15/11; A61K 9/14; A61K 47/54; C12N 9/22

(52) U.S. Cl. CPC C12N 15/11; A61K 9/14; A61K 47/543; C12N 9/22

(57) ABSTRACT

The present disclosure relates to a nucleic acid nanoparticle comprising a single stranded nucleic acid scaffold encoding one or more RNA molecules of interest and a plurality of single stranded nucleic acid oligomers, where each oligomer is complementary to at least a portion of the single stranded nucleic acid scaffold. Also disclosed are pharmaceutical compositions and methods of use thereof.

### PRIORITY

This application claims the priority benefit of U.S. Provisional Patent Application Ser. No. 63/447,258, filed Feb. 21, 2023, which is hereby incorporated by reference in its entirety.

### SEQUENCE STATEMENT

The Sequence Listing was submitted electronically in XML format and is hereby incorporated by reference in its entirety. Said XML copy, created on Feb. 21, 2024, is named 147426001471.xml and is 276,023 bytes in size. No new matter is introduced.

### FIELD

The present disclosure relates to nucleic acid nanoparticles, as well as pharmaceutical compositions and methods of their use.

### BACKGROUND

The worldwide outbreak of COVID-19 has accelerated vaccine development and implementation at an unprecedented rate. Consequently, enormous investment has been made by governmental and independent agencies while, in parallel, the FDA has accelerated the clinical trial process. Most importantly, rapid vaccine development has led to rapid implementation of novel technologies such as DNA and mRNA-based vaccines (Li et al., “Coronavirus Vaccine Development: From SARS and MERS to COVID-19,” Journal of Biomedical Science 27:104 (2020)). Despite the clear (albeit possibly transient) mitigation of the COVID-19 outbreak owing to rapid distribution of DNA and mRNA-based vaccines, current DNA and mRNA-based vaccines still retain several liabilities and can, therefore, be further improved. With the advent of CRISPR-based gene editing, it may be possible to modify genomes of infectious agents or their hosts to mitigate infection and treat or cure genetic disease.

DNA Vaccines. DNA-based vaccines provide a highly adaptable platform for vaccine development for novel pathogens and known pathogen mutants due to the rapid, flexible, and relatively economical nature of this methodology (Li et al., “Coronavirus Vaccine Development: From SARS and MERS to COVID-19,” Journal of Biomedical Science 27:104 (2020)). However, there are several drawbacks to this approach. First, most DNA-based vaccines rely on viral vectors for in vivo delivery. Although a number of viral vectors such as adenovirus vectors, ensures safe and efficient delivery, viral vector mediated DNA delivery carries the risk of genomic integration which could result in undesirable genomic alterations as severe as mutagenesis and oncogenesis. The production requirements to minimize (but not totally eliminate) this risk are costly and introduce steps that are inherently possible sources of error (Li et al., “Coronavirus Vaccine Development: From SARS and MERS to COVID-19,” Journal of Biomedical Science 27:104 (2020)). A second liability of this platform is that preexisting immunity may decrease transfection efficiency of viral vectors (Li et al., “Coronavirus Vaccine Development: From SARS and MERS to COVID-19,” Journal of Biomedical Science 27:104 (2020); Zheng et al., “Lentiviral Vectors and Adeno-Associated Virus Vectors: Useful Tools for Gene Transfer in Pain Research,” The Anatomical Record 301:825-836 (2018); and Yin et al., “Delivery Technologies for Genome Editing,” Nature Reviews Drug Discovery 16:387-399 (2017)).

Messenger RNA (mRNA) Vaccines. mRNA-based vaccines also provide the benefit of a highly adaptable vaccine development and production platform and, unlike DNA-based vaccines, mRNA-based vaccines, typically configured as lipid nanoparticles, carry minimal risk of genomic integration in contrast to virally encapsulated DNA vaccines (Li et al., “Coronavirus Vaccine Development: From SARS and MERS to COVID-19,” Journal of Biomedical Science 27:104 (2020); Zhang et al., “Advances in mRNA Vaccines for Infectious Diseases,” Frontiers in Immunology 10:594 (2019); and Pardi et al., “mRNA Vaccines-A New Era in Vaccinology,” Nature Reviews Drug Discovery 17:261-279 (2018)). Unfortunately, despite the enhanced safety of mRNA-based vaccines, they commonly suffer from temperature sensitivity and instability (Zheng et al., “Lentiviral Vectors and Adeno-Associated Virus Vectors: Useful Tools for Gene Transfer in Pain Research,” The Anatomical Record 301:825-836 (2018)). Natural RNA is far less chemically stable than DNA, and an abundance of RNAse enzymes in living systems makes mRNA susceptible to rapid degradation (Zhang et al., “Advances in mRNA Vaccines for Infectious Diseases,” Frontiers in Immunology 10:594 (2019); Pardi et al., “mRNA Vaccines-A New Era in Vaccinology,” Nature Reviews Drug Discovery 17:261-279 (2018); Xu et al., “mRNA Vaccine Eri-Mechanisms, Drug Platform and Clinical Prospection,” Molecular Sciences 21:6582 (2020); and Maruggi et al., “mRNA as a Transformative Technology for Vaccine Development to Control Infectious Diseases,” Molecular Therapy 27(4): 757-772 (2019)). Thus, the most successful mRNA-based vaccines require enhancements such as chemical modification, nanoparticle configuration, and lipid coupling to enhance stability, intracellular delivery, and half-life of the mRNA in vivo (Zhang et al., “Advances in mRNA Vaccines for Infectious Diseases,” Frontiers in Immunology 10:594 (2019); Pardi et al., “mRNA Vaccines-A New Era in Vaccinology,” Nature Reviews Drug Discovery 17:261-279 (2018); Wadhwa et al., “Opportunities and Challenges in the Delivery of mRNA-Based Vaccines,” Pharmaceutics 12:102 (2020); Wang et al., “mRNA Vaccines: A Potential Therapeutic Strategy,” Molecular Cancer 20:33 (2021); Xu et al., “mRNA Vaccine Eri-Mechanisms, Drug Platform and Clinical Prospection,” Molecular Sciences 21:6582 (2020); Maruggi et al., “mRNA as a Transformative Technology for Vaccine Development to Control Infectious Diseases,” Molecular Therapy 27(4): 757-772 (2019); and Borah et al., “Perspectives on RNA Vaccine Candidates for COVID-19,” Frontiers in Molecular Biosciences 8:635245 (2021).

The present invention is directed to overcoming these and other deficiencies in the art.

### SUMMARY

A first aspect of the present disclosure relates to a nucleic acid nanoparticle comprising a single stranded nucleic acid scaffold encoding one or more RNA molecules and/or proteins of interest and a plurality of single stranded nucleic acid oligomers, where each oligomer is complementary to at least a portion of the single stranded nucleic acid scaffold.

Another aspect of the present disclosure relates to a pharmaceutical composition comprising a nucleic acid nanoparticle according to the present disclosure and a pharmaceutically acceptable carrier.

Another aspect of the present disclosure relates to a method of eliciting an immune response in a subject. This method involves administering a nucleic acid nanoparticle or a pharmaceutical composition according to the present disclosure to a subject, where said administering is effective to elicit an immune response in the subject.

Another aspect of the present disclosure relates to a method of expressing a heterologous RNA molecule and/or protein of interest in a population of cells. This method involves contacting a population of cells with a nucleic acid nanoparticle according to the present disclosure.

Self-assembling nucleic acid nanoparticles (i.e., DNA nanoparticles) provide an architecturally and chemically malleable platform for a wide range of applications (Kearney et al., “DNA Origami: Folded DNA-Nanodevices That Can Direct and Interpret Cell Behavior,” Advanced Materials 28(27):5509-5524 (2016); Ora et al., “Cellular Delivery of Enzyme-Loaded DNA Origami,” Chemical Communications 52(98): 14161-14164 (2016); Ramakrishnan et al., “Structural Stability of DNA Origami Nanostructures under Application-Specific Conditions,” Computat. Struct. Biotechnol. J. 16:342-349 (2018); Mathur and Medintz, “Analyzing DNA Nanotechnology: A Call to Arms for the Analytical Chemistry Community,” Anal. Chem. 89(5):2646-2663 (2017); Wang et al., “DNA Origami Protection and Molecular Interfacing through Engineered Sequence-Defined Peptoids,” PNAS 117(12):6339-6348 (2020); Daljit et al., “Switchable DNA-Origami Nanostructures that Respond to their Environment and their Applications,” Biophys. Rev. 10(5): 1283-1293 (2018); Wang et al., “The Beauty and Utility of DNA Origami,” Chem. 2:359-382 (2017); and Coleridge and Dunn, “Assessing the Cost-Effectiveness of DNA Origami Nanostructures for Targeted Delivery of Anti-Cancer Drugs to Tumours,” Biomed Phys. Eng. Express 6(6) (2020), which are hereby incorporated by reference in their entirety). Exemplary uses of DNA nanoparticles include targeted drug delivery (Kearney et al., “DNA Origami: Folded DNA-Nanodevices That Can Direct and Interpret Cell Behavior,” Advanced Materials 28(27):5509-5524 (2016); Ora et al., “Cellular Delivery of Enzyme-Loaded DNA Origami,” Chemical Commun. 52(98): 14161-14164 (2016); Wang et al., “DNA Origami Protection and Molecular Interfacing through Engineered Sequence-Defined Peptoids,” PNAS 117(12):6339-6348 (2020); Wang et al., “The Beauty and Utility of DNA Origami,” Chem. 2:359-382 (2017); Burns et al., “DNA Origami Inside-Out Viruses,” ACS Synth. Biol. 7(3): 767-773 (2018); Li et al., “A DNA Nanorobot Functions as a Cancer Therapeutic in Response to a Molecular Trigger in vivo,” Nat. Biotechnol. 36:258-264 (2018); Brglez et al., “Designed Intercalators for Modification of DNA Origami Surface Properties,” Chemistry 21(26):9440-9446 (2015); Chi et al., “DNA Nanostructure as an Efficient Drug Delivery Platform for Immunotherapy,” Front. Pharmacol. 10:1585 (2020); and Dobrovolskaia and Bathe, “Opportunities and Challenges for the Clinical Translation of Structured DNA Assemblies as Gene Therapeutic Delivery and Vaccine Vectors,” Wiley Interdisciplinary Rev. Nanomed. Nanobiotechnol. 13(1):e1657 (2021), which are hereby incorporated by reference in their entirety), biosensing (Kearney et al., “DNA Origami: Folded DNA-Nanodevices That Can Direct and Interpret Cell Behavior,” Advanced Materials 28(27):5509-5524 (2016); Ramakrishnan et al., “Structural Stability of DNA Origami Nanostructures under Application-Specific Conditions,” Computat. Struct. Biotechnol. J. 16:342-349 (2018); Wang et al., “DNA Origami Protection and Molecular Interfacing through Engineered Sequence-Defined Peptoids,” PNAS 117(12):6339-6348 (2020); Wang et al., “The Beauty and Utility of DNA Origami,” Chem. 2:359-382 (2017); and Madsen and Gothelf, “Chemistries for DNA Nanotechnology,” Chem. Rev. 119(10):6384-6458 (2019), which are hereby incorporated by reference in their entirety), nanoelectronics (Ramakrishnan et al., “Structural Stability of DNA Origami Nanostructures under Application-Specific Conditions,” Computat. Struct. Biotechnol. J. 16:342-349 (2018); Daljit et al., “Switchable DNA-Origami Nanostructures that Respond to their Environment and their Applications,” Biophys. Rev. 10(5): 1283-1293 (2018); Ouyang et al., “Rolling Circle Amplification-Based DNA Origami Nanostructures for Intracellular Delivery of Immunostimulatory Drugs,” Small 9(18):3082-3087 (2013); and Hannewald et al., “DNA Origami Meets Polymers: A Powerful Tool for the Design of Defined Nanostructures,” Angew. Chem. Int. Ed. Engl. 60(12):6218-6229 (2020), which are hereby incorporated by reference in its entirety), bioimaging (Kearney et al., “DNA Origami: Folded DNA-Nanodevices That Can Direct and Interpret Cell Behavior,” Advanced Materials 28(27):5509-5524 (2016), which is hereby incorporated by reference in its entirety), nanorobotics (Ora et al., “Cellular Delivery of Enzyme-Loaded DNA Origami,” Chemical Communications 52(98): 14161-14164 (2016); Ramakrishnan et al., “Structural Stability of DNA Origami Nanostructures under Application-Specific Conditions,” Computat. Struct. Biotechnol. J. 16:342-349 (2018); Wang et al., “DNA Origami Protection and Molecular Interfacing through Engineered Sequence-Defined Peptoids,” PNAS 117(12):6339-6348 (2020); and Ouyang et al., “Rolling Circle Amplification-Based DNA Origami Nanostructures for Intracellular Delivery of Immunostimulatory Drugs,” Small 9(18):3082-3087 (2013), which are hereby incorporated by reference in their entirety), nanophotonics (Wang et al., “DNA Origami Protection and Molecular Interfacing through Engineered Sequence-Defined Peptoids,” PNAS 117(12):6339-6348 (2020); Wang et al., “The Beauty and Utility of DNA Origami,” Chem. 2:359-382 (2017); Madsen and Gothelf, “Chemistrics for DNA Nanotechnology,” Chem. Rev. 119(10):6384-6458 (2019); and Hannewald et al., “DNA Origami Meets Polymers: A Powerful Tool for the Design of Defined Nanostructures,” Angew. Chem. Int. Ed. Engl. 60(12):6218-6229 (2020), which are hereby incorporated by reference in their entirety), CpG triggered immunostimulation (Ora et al., “Cellular Delivery of Enzyme-Loaded DNA Origami,” Chemical Communications 52(98): 14161-14164 (2016); Wang et al., “DNA Origami Protection and Molecular Interfacing through Engineered Sequence-Defined Peptoids,” PNAS 117(12):6339-6348 (2020); Dobrovolskaia and Bathe, “Opportunities and Challenges for the Clinical Translation of Structured DNA Assemblies as Gene Therapeutic Delivery and Vaccine Vectors,” Wiley Interdisciplinary Rev. Nanomed. Nanobiotechnol. 13(1):c1657 (2021); and Ouyang et al., “Rolling Circle Amplification-Based DNA Origami Nanostructures for Intracellular Delivery of Immunostimulatory Drugs,” Small 9(18):3082-3087 (2013), which are hereby incorporated by reference in their entirety), synthetic nanopore formation (Kearney et al., “DNA Origami: Folded DNA-Nanodevices That Can Direct and Interpret Cell Behavior,” Advanced Materials 28(27):5509-5524 (2016), which is hereby incorporated by reference in its entirety), and templates for in vivo genomic integration and subsequent protein expression (Lin-Shiao et al., “CRISPR-Cas9-Mediated Nuclear Transport and Genomic Integration of Nanostructured Genes in Human Primary Cells,” Nucleic Acids Res. 50(3): 1256-1268 (2022), which is hereby incorporated by reference in its entirety). The embedded biological code in DNA makes sculpted DNA nanoparticles particularly attractive for uses in diagnostics and therapeutics. Although DNA is inherently immunogenic, it can be chemically modified to tune its biocompatibility (Kearney et al., “DNA Origami: Folded DNA-Nanodevices That Can Direct and Interpret Cell Behavior,” Advanced Materials 28(27):5509-5524 (2016); Ora et al., “Cellular Delivery of Enzyme-Loaded DNA Origami,” Chemical Communications 52(98): 14161-14164 (2016); Ramakrishnan et al., “Structural Stability of DNA Origami Nanostructures under Application-Specific Conditions,” Computat. Struct. Biotechnol. J. 16:342-349 (2018); Wang et al., “The Beauty and Utility of DNA Origami,” Chem. 2:359-382 (2017); and Coleridge and Dunn, “Assessing the Cost-Effectiveness of DNA Origami Nanostructures for Targeted Delivery of Anti-Cancer Drugs to Tumours,” Biomed Phys. Eng. Express 6(6) (2020), which are hereby incorporated by reference in their entirety). Thus, it may be feasible to employ self-assembling DNA nanoparticles as a platform to ferry biologically active, intentionally camouflaged gene cassettes to specific locations in cell culture and even in vivo wherein, they may be expressed without the requirement of chromosomal insertion (Lin-Shiao et al., “CRISPR-Cas9-Mediated Nuclear Transport and Genomic Integration of Nanostructured Genes in Human Primary Cells,” Nucleic Acids Res. 50(3):1256-1268 (2022), which is hereby incorporated by reference in its entirety). Such a delivery system would be useful in the context of molecular therapeutics (e.g., targeted gene therapy), especially when a transient expression is desirable. As a first step toward this goal, the ability of gene-bearing DNA nanoparticles constructed by the method of DNA origami (Oh and Henderson, “A Comparison of Methods for the Production of Kilobase-Length Single-Stranded DNA,” DNA 2(1):56-67 (2022), which is hereby incorporated by reference in its entirety) to undergo gene expression in vitro in test tube and cell culture systems is reported herein. Further, the examples of the present disclosure demonstrate that while cells possess the remarkable capability to express genes within highly folded architectures, several parameters, such as the concurrent folding of promoter/enhancer and gene, the presence of ssDNA domains, and even the buffer composition where the DNA nanoparticles are suspended and significantly influence overall levels of gene expression.

### BRIEF DESCRIPTION OF THE DRAWINGS

FIG. 1 is an illustration of one embodiment of a gene construct used herein and the process of scaffold and DNA nanoparticle generation via aPCR and DNA origami, respectively, along with a schematic diagram of different DNA nanoparticles used. Primary variations include the number/position of crossovers in the origami architecture and relative accessibility of the T7 RNA polymerase promoter region; the T7 promoter being located either on a linear duplex extending from the body of the nanoparticle, embedded within the nanoparticle, or absent altogether.

FIGS. 2A-2B show agarose gel electrophoretic (AGE) result of DNA nanoparticle production. FIG. 2A demonstrates ssDNA production. Representative non-denaturing agarose gel electrophoretic results of PCR and aPCR. M: molecular marker; 1: GFP plasmid template for PCR (pCMV-T7-EGFP); 2: unpurified PCR product; 3: purified PCR product (dsT7EGFP); 4: unpurified aPCR product; 5: purified aPCR product (ssT7EGFP). The bands at approximately 3 kb, 1.7 kb, and 0.9 kb represent GFP plasmid, double-stranded GFP gene, and single-stranded GFP genc, respectively. FIG. 2B demonstrates the generation of DNA nanoparticles from GFP gene-containing scaffolds±the T7 promoter; ssT7EGFP and ssT7EGFP-T7. Representative agarose gel electrophoresis display of the DNA nanoparticles used in work described in the Examples. M: molecular marker; 1: dsT7EGFP; 2: ssT7EGFP; 3: T7GHL PO (DNA nanoparticle with a linear duplex promoter and single-stranded gene); 4: T7GHL HS (DNA nanoparticle with a linear duplex promoter and partially folded gene with half a set of staples); 5: T7GHL FS (DNA nanoparticle with a linear duplex promoter and a full set of staples); 6: T7GHL BP (DNA nanoparticle with a linear duplex promoter buried inside the fully folded genc); 7: dsT7EGFP-T7 (double-stranded GFP genc without T7 promoter); 8: ssT7EGFP-T7 (single-stranded GFP gene without T7 promoter); 9: T7GHL-T7 (fully folded DNA nanoparticle without T7 promoter). For lanes 1 and 2, the band at approximately 1.7 kb represents dsT7EGFP, and the smear that runs from approximately 0.7 kb to 0.3 kb represents ssT7EGFP. The bands around 1 kb for lanes 3, 4, 5, and 6 represent successfully self-assembled DNA nanoparticles. For lanes 7 and 8, the band at approximately 1.1 kb represents dsT7EGFP-T7 and the smear that runs from 0.5 kb to 0.4 kb represents ssT7EGFP-T7. The band at approximately 0.6 kb in Lane 9 represents the successfully self-assembled DNA nanoparticle lacking the T7 promoter.

FIG. 3 verifies the structural integrity of gel-purified DNA nanoparticles by transmission electron microscopy (TEM). A representative DNA nanoparticle indicating key structural elements to the lower right of each representative TEM field. Single-stranded and loosely crosslinked domains appear as relatively amorphous consolidated networks whereas linear duplexes containing the T7 promoter appear as curved tails due to the greater persistence length of double-stranded DNA relative to single-stranded DNA (Yang et al., “A Single Strand: A Simplified Approach to DNA Origami,” Front. Chem. 11:1126177 (2023), which is hereby incorporated by reference in its entirety). Compact, highly crosslinked origami regions display the canonical multi-helix particle morphology.

FIG. 4 demonstrates the in vitro transcription of DNA nanoparticles. Representative non-denaturing agarose gel electrophoresis results of in vitro transcription reactions. M: molecular marker; ss: ssT7EGFP; ds: dsT7GFP; Lp: LpCMV-T7-EGFP (linearized GFP plasmid); PO: T7GHL PO; HS: T7GHL HS; FS: T7GHL FS; BP: T7GHL BP;-T7: T7GHL-T7. LpCMV-T7-EGFP and dsT7EGFP were positive controls. ssT7EGFP, T7GHL BP, and T7GHL-T7 were putative negative controls. The bands at approximately 0.9 kb, 1.1 kb, and 1.3 kb represent GFP RNA. The 0.9 kb band represents transcripts that terminate at a T7 RNA polymerase termination/stall sequence, known to allow a significant amount of read-through (Lyakhov et al., “Pausing and Termination by Bacteriophage T7 RNA Polymerase,” J. Mol. Biol. 280(2):201-213 (1998) and Song and Kang, “Sequence-Specific Termination by T7 RNA Polymerase Requires Formation of Paused Conformation prior to the Point of RNA Release,” Genes Cells 6(4):291-301 (2001), which is hereby incorporated by reference in its entirety). The 1.1 kb and 1.3 kb RNA products represent read-through transcripts, the latter's greater length resulting from the linearized plasmid being 200 bp longer than the PCR-generated linear duplex. The bands of approximately 2.5 kb for most of the samples and 1.5 kb in the T7GHL-T7 lane result from the presence of residual DNA templates.

FIG. 5 verifies RNA production by RT-PCR. Representative agarose gel electrophoresis results of RT-PCR. M: molecular marker. +RT: Reverse transcription of RNA products followed by PCR,-RT: PCR of IVT products without reverse transcription of RNA products. The bands around 0.7 kb represent the amplified GFP gene. GFP bands in-RT lanes reflect the amplification of residual DNA that persists after DNase treatment. Regardless of presence of GFP bands on both +RT and −TR lanes, brighter band intensities in +RT lanes support the successful reverse transcription, meaning the presence of GFP RNA products; resultants of the IVT.

FIG. 6 shows an IVT time course with various nanoparticle constructs. Representative non-denaturing agarose gel electrophoresis results of an IVT time course with DNA nanoparticles (T7GHL PO, T7GHL HS, T7GHL FS, and T7GHL BP). M: molecular marker. The bands around 1.1 kb and 0.9 kb represent GFP RNA. The band above the RNA bands (i.e., approximately 2.5 kb) represents the DNA template. For each DNA nanoparticle, IVT reactions were carried out for 30, 60, 90, and 120 minutes. All DNA nanoparticles used for the time course produced RNA at the earliest time period (30 minutes), and production of RNA persisted and increased to the 120-minute time point.

FIG. 7 provides the caDNAno designs of various DNA nanoparticles (GFP gene with T7 promoter with Honeycomb Loop shape structure (T7GHL)). T7GHL PO: DNA nanoparticle with a linear duplex promoter and single-stranded gene; T7GHL HS: DNA nanoparticle with a linear duplex promoter and partially folded gene with half a set of staples; T7GHL FS: DNA nanoparticle with a linear duplex promoter and fully folded gene; T7GHL-T7: fully folded DNA nanoparticle missing T7 promoter; T7GHL BP: DNA nanoparticle with a linear duplex promoter buried inside the fully folded gene. The colors on 3D models indicate relative flexibility within DNA nanoparticles. Red and blue indicate high and low flexibility respectively, and the color ranging in between correlates with the flexibility.

FIG. 8 shows a representative agarose gel electrophoresis result verifying the structural integrity of DNA nanoparticle under IVT reaction buffer and temperature with increasing Mg²⁺ concentration. M: molecular marker; 1: T7GHL FS; 2: Treatment of T7GHL FS under IVT reaction buffer and temperature with an additional 0.9 mM Mg²⁺total; 3: Treatment of T7GHL FS under IVT reaction buffer and temperature T7GHL FS with an additional 1.9 mM Mg²⁺total; 4: Treatment of T7GHL FS under IVT reaction buffer and temperature T7GHL FS with an additional 3.8 mM Mg²⁺total; 5: Treatment of T7GHL FS under IVT reaction buffer and temperature T7GHL FS with an additional 5 mM Mg²⁺total. The bands around 1 kb represent T7GHL FS. The result indicates a minimal shift in DNA nanoparticle migrations suggesting that DNA nanoparticles retain their structural integrities under IVT reaction conditions.

FIG. 9 shows a representative agarose gel electrophoresis result of products of dsT7EGFP and T7GHL BP IVT with increasing Mg²⁺ concentrations. M: molecular marker; 1: IVT on dsT7EGFP; 2: IVT on dsT7EGFP with an addition of 1.6 mM Mg²⁺; 3: IVT on dsT7EGFP with an addition of 3.2 mM Mg²⁺; 4: IVT on dsT7EGFP with an addition 5 mM Mg²⁺; 5: IVT on T7GHL BP with an addition 1.6 mM Mg²⁺; 6: IVT on dsT7EGFP with an addition 3.2 mM Mg²⁺; 7: IVT on dsT7EGFP with an addition 5 mM Mg²⁺. The bands around 1.1 kb and 0.9 kb represent GFP RNA. Intensities of the bands pre- and post-treatment of additional Mg²⁺to IVT reactions do not show noticeable differences. Additional magnesium ions were added to IVT reactions to ensure structural integrities of DNA nanoparticles prior to the initiation of the transcription. The result suggests that T7GHL BP remains intact prior to the initiation of the transcription, which further suggests that the transcription of T7GHL BP occurs without pre-disintegration of the nanoparticle structure.

FIG. 10 shows a representative agarose gel electrophoretic result of RT-PCR products on IVT products with increased DNase treatment time. M: molecular marker; +RT 0′: RT-PCR product on IVT with 0 minutes DNase treatment;-RT 0′: PCR product on IVT with 0 minutes DNase treatment (no RT); +RT 5′: RT-PCR product on IVT with 5 minutes DNase treatment;-RT 5′: PCR product on IVT with 5 minutes DNase treatment (no RT); +RT 15′: RT-PCR product on IVT with 15 minutes DNase treatment;-RT 15′: PCR product on IVT with 15 minutes DNase treatment (no RT); +RT 30′: RT-PCR product on IVT with 30 minutes DNase treatment;-30RT 30′: PCR product on IVT with 30 minutes DNase treatment (no RT). The bands around 0.7 kb represent the GFP gene. The intensities of GFP bands on-RT lanes decrease upon increased duration of DNase treatment whereas the intensities of GFP bands on +RT lanes remained noticeably bright even upon increased duration of DNase treatment. This supports that background noise on-RT lanes are caused by undegraded full-length DNA template that survived after DNase treatment.

FIGS. 11A-11B demonstrate caDNAno designs of various DNA nanoparticles. FIG. 11A provides the design of DNA nanoparticles with linear promoter/enhancer (GFP gene Honeycomb structure with a Linear promoter and enhancer; GHL). GHL PO: DNA nanoparticle with linear promoter/enhancer and single-stranded gene; GHL HS: DNA nanoparticle with linear promoter/enhancer and a half complement of staples; GHL FS: DNA nanoparticle with linear promoter/enhancer and a mostly full complement of staples; GHL FSC: DNA nanoparticle with linear promoter/enhancer and a full complement of staples. FIG. 11B provides the design of DNA nanoparticles with folded promoter/enhancer (GFP gene Honeycomb structure with a Honeycomb-loop folded promoter and enhancer; GHH). GHH: DNA nanoparticle with loosely folded promoter/enhancer and tightly folded gene; GHH LB: DNA nanoparticle with loosely folded promoter/enhancer and linear duplex gene; GHH CP: DNA nanoparticle with tightly folded promoter/enhancer and genc.

FIG. 12 demonstrates the generation and purification of DNA nanoparticles from GFP gene-containing scaffolds. Representative agarose gel electrophoresis display of the purified DNA nanoparticles disclosed herein. M: molecular marker; 1: dsGFP (fully duplex GFP genc); 2: ssGFP (single-stranded antisense strand (i.e., scaffolds for DNA nanoparticles)); GHL: DNA nanoparticles with linear promoter/enhancer; 3: PO (GHL PO; DNA nanoparticle with linear promoter/enhancer and single-stranded genc); 4: HS (GHL HS; DNA nanoparticle with linear promoter/enhancer and a half complement of staples); 5: FS (GHL FS; DNA nanoparticle with linear promoter/enhancer and a mostly full complement of staples); 6: FSC (GHL FSC; DNA nanoparticle with linear promoter/enhancer and a full complement of staples); GHH: DNA nanoparticles with folded promoter/enhancer; 7: HH (GHH); 8: LB (GHH LB; DNA nanoparticle with loosely folded promoter/enhancer and linear duplex gene); 9: CP (GHH CP; DNA nanoparticle with tightly folded promoter/enhancer and gene). For lanes 1 and 2, the band at approximately 1.7 kb represents dsGFP, and the band at approximately 0.7 kb represents ssGFP. The bands between 1.7 kb and 1 kb for lanes 3, 4, 5, and 6 represent successfully self-assembled DNA nanoparticles with linear duplex promoter/enhancer. The smear that runs from the well to 0.5 kb in lane 7, bands at approximately 3 kb and 1.5 kb on lane 8, and the band around 0.9 kb on lane 9 represent self-assembled DNA nanoparticles with either loosely or tightly folded promoter/enhancer.

FIGS. 13A-13B verify the structural integrity of ultracentrifugation-filtered DNA nanoparticles by transmission electron microscopy (TEM). A representative DNA nanoparticle with key structural elements is located to the lower right of each representative TEM field. ssDNA and loosely crosslinked domains appear as relatively amorphous consolidated networks whereas linear duplexes containing the promoter and enhancer appear as curved tails due to the increased persistence length of dsDNA relative to ssDNA (Yang et al., “A Single Strand: A Simplified Approach to DNA Origami,” Front. Chem. 11:1126177 (2023), which is hereby incorporated by reference in its entirety). Compact, highly crosslinked origami regions display the canonical multi-helix particle morphology. FIG. 13A shows GHL PO, GHL HS, GHL FS, and GHL FSC nanoparticles. FIG. 13B shows GHH, GHH LB, and GHH CP nanoparticles.

FIGS. 14A-14D are representative images of cultured cells expressing GFP upon transfection with various DNA nanoparticles. The green color, regardless of intensity, represents cells expressing GFP. FIG. 14A shows cultured cells transfected with dsGFP (positive control) to obtain the maximum degree of GFP expression. FIG. 14B shows cultured cells transfected with ssGFP (bare scaffold) as a negative control. FIG. 14C shows cultured cells transfected with DNA nanoparticles with linear duplex promoter/enhancer. There is an apparent inverse relationship between the degree of single-strandedness and GFP expression. FIG. 14D shows cultured cells transfected with DNA nanoparticles with a folded promoter/enhancer domain. GFP expression was observed regardless of how tightly folded (i.e., staple density) the promoter and enhancer were with the most densely stapled architecture having the highest level of expression.

FIG. 15 is a graphical representation of EGFP expression statistics from various DNA samples. GFP expression was measured by counting the number of GFP-positive cells. The percentage of GFP expression for each DNA sample was calculated and adjusted relative to a positive control (dsGFP). The average relative percentage of each sample is represented as a bar. Error bars represent one standard deviation from the mean of three images and each dot represents a data point obtained from each image taken.

FIGS. 16A-16K are representative flow cytometry plots of cultured cells expressing GFP upon transfection with various DNA nanoparticles. DNA nanoparticles were assembled in TAEM buffer which was exchanged with phosphate buffered saline (PBS) during the nanoparticle purification process. Cells were gated based on size and granularity using FSC-A vs SSC-A. From the “cells” gate, single cells were sub-gated by their FSC-A vs. FSC-H properties. Single cells were evaluated for EGFP expression. FIGS. 16A-16K show data for blank cells (negative control; FIG. 16A), dsGFP (FIG. 16B), dsGFP-a (FIG. 16C), ssGFP (FIG. 16D), GHL PO (FIG. 16E), GHL HS (FIG. 16F), GHL FS (FIG. 16G), GHL FSC (FIG. 16H), GHH (FIG. 16I), GHH LB (FIG. 16J), and GHH CP (FIG. 16K). SSC-A: side scatter area; FSC-A: forward scatter area; FSC-H: forward scatter height. (GFP) FITC-A: green emission (500-550 nm) excited from the 488 nm laser; GFP signal area measurement.

FIG. 17 is a graphical representation of the statistics obtained from flow cytometry data in FIGS. 16B-16K. FIG. 17 shows the relative percentage of EGFP expression adjusted based on the level of expression measured by positive control (dsGFP) as well as baseline measured through negative control (blank cells) corresponding to the flow cytometry data shown for dsGFP-a (FIG. 16C), ssGFP (FIG. 16D), GHL PO (FIG. 16E), GHL HS (FIG. 16F), GHL FS (FIG. 16G), GHL FSC (FIG. 16H), GHH (FIG. 16I), GHH LB (FIG. 16J), and GHH CP (FIG. 16K), relative to the EGFP expression corresponding to the flow cytometry data shown for dsGFP (FIG. 16B).

### DETAILED DESCRIPTION

### Definitions

Unless otherwise indicated, the definitions and embodiments described in this and other sections are intended to be applicable to all embodiments and aspects of the present disclosure herein described for which they are suitable as would be understood by a person skilled in the art.

Singular forms “a”, “an”, and “the” include plural references unless the context clearly dictates otherwise. Thus, for example, a reference to “a method” includes one or more methods and/or steps of the type described herein and/or which will become apparent to a person of ordinary skill in the art upon reading this disclosure. In another example, reference to “a compound” includes both a single compound and a plurality of different compounds.

The term “about” or “approximately” includes being within a statistically meaningful range of a value. Such a range can be within an order of magnitude, such as within 50%, or within 20%, or within 10%, or within 5% (or any amount or range within 5-50%) of a given value or range. The allowable variation encompassed by the term “about” or “approximately” may depend on the context.

The term “and/or” as used herein means that the listed features are present, or used, individually or in combination. In effect, this term means that “at least one of” or “one or more” of the listed features is used or present.

The term “genetically active” as used herein refers to nucleic acid molecule encoding a gene which is capable of being recognized and acted upon by molecular systems responsible for regulation and expression and of genes and gene controlling and modifying elements including but not limited to DNA, RNA, protein, and all controlling sequence elements including, but not limited to, promoters, enhancers, epigenetic modification sites, and poly-A tails. The term “genetically inactive” refers to a nucleic acid molecule encoding a gene which is incapable of being recognized or acted upon by molecular systems responsible for regulation and expression and of genes and gene controlling and modifying elements including but not limited to DNA, RNA, protein, and all controlling sequence elements.

As will be understood by a person of ordinary skill in the art, for any and all purposes, such as in terms of providing a written description, all ranges disclosed herein also encompass any and all possible subranges and combinations of subranges thereof, as well as any value within a range. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by a person of ordinary skill in the art all language such as “up to,” “at least,” and the like include the number recited and refer to ranges which can be subsequently broken down into subranges or specific values therein as discussed above. Finally, as will be understood by a person of ordinary skill in the art, and as discussed above, a range includes each individual value.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

In some embodiments comprising an “additional” or “second” component, the second component as used herein is different from the other components or first component. A “third” component is different from the other, first, and second components, and further enumerated or “additional” components are similarly different.

The term “self-assembly” refers to the process by which nucleic acid molecules may assemble into specific architecture based on sequence complementary interactions between the components of the nucleic acid molecules. For example, nucleic acid nanoparticles may self-assemble (in the absence of external information or user intervention) once all the components of the desired nucleic acid nanoparticle have been mixed. The entire set of instructions for creating the desired nanosystem is extant within the molecular ensemble constituting the mixture.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present disclosure, some embodiments of the methods and materials are now described. All publications mentioned herein are incorporated herein by reference to disclose and describe the methods and/or materials in connection with which the publications are cited.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### DNA Origami

One strategy for building complex DNA assemblies is to use a process termed “DNA Origami” (see, e.g., Rothemund et al., “Folding DNA to Create Nanoscale Shapes and Patterns,” Nature 440:297-302 (2006), which is hereby incorporated by reference in its entirety). In this process, a large single-stranded DNA molecule (the “scaffold”, usually, but not necessarily, a genome obtained from the bacterial virus (bacteriophage) M13 or similar) is mixed with a large number (approximately 200 is typical, though this number is highly variable) of small synthetic DNA molecules that form double helices (dsDNA) connected by crossovers related to a junction observed in DNA recombination events (Holiday junction). The small synthetic DNA molecules are called “staples” or “helper strands”, and they tie together distal (nonadjacent) portions of the large single-stranded “scaffold” strand into the desired shape. The analogy to folding paper to create shapes (origami) led to the term DNA origami. It is noteworthy that processes for accomplishing this process without the M13 scaffold, entirely from small molecules, has been demonstrated (Mathur and Henderson, “Complex DNA Nanostructures from Oligonucleotide Ensembles,” ACS Synthetic Biology 2(4): 180-185 (2013), which is hereby incorporated by reference in its entirety) because this method eliminates the need for a single-stranded scaffold and, therefore, creates many more avenues to create DNA nanosystems and to build multiple devices in a single reaction. One can leave portions of the DNA single stranded (ssDNA) and in this way create a structure that has one conformation but will change conformation when the synthetic DNA complementary to the remaining single stranded regions are added. This ssDNA>dsDNA conversion is the basis for operation of the systems and methods used in biosensing DNA nanosystems due to the fact that such transitions generate forces (pushing or pulling) in a user-defined vector orientation (Mathur and Henderson, “Programmable DNA Nanosystem for Molecular Interrogation,” Nature Scientific Reports 6:27413 (2016), which is hereby incorporated by reference in its entirety). In other words, by adding DNA to convert ssDNA to dsDNA, the user can pull on specific molecular pairs and measure the forces holding them together.

### Nucleic Acid Nanoparticles

Self-assembling nucleic acid nanoparticles provide a novel and promising platform for in vitro and in vivo development and optimization of potential nucleic acid nanoparticle vaccines, and in vivo delivery of genetic material, in this instance, a gene or genes encoding vaccine components. A variety of methods have emerged that leverage the self-assembly code embedded in nucleic acids. These include DNA brick (Rogers et al., “Using DNA to Program the Self-Assembly of Colloidal Nanoparticles and Microparticles,” Nat. Rev. Mat. 1:16008 (2016); Ke et al., “Three-Dimensional Structures Self-Assembled from DNA Bricks,” Science 338(6111): 1177-1183 (2012); Green et al., “Autonomous Dynamic Control of DNA Nanostructure Self-Assembly,” Nature Chem. 11:510-520 (2019); Gradišar et al., “Self-Assembled Bionanostructures: Proteins Following the Lead of DNA Nanostructures,” J. Nanobiotechnology 12:4 (2014); Bujold et al., “DNA Nanostructures at the Interface with Biology,” Chem. 4(3):495-521 (2018), which are hereby incorporated by reference in their entirety), DNA origami (Rogers et al., “Using DNA to program the self-assembly of colloidal nanoparticles and microparticles,” Nature Reviews Materials 1:16008 (2016); Zhang et al., “DNA self-assembly scaled up,” Nature 552:34-35 (2017); Ke et al., “Three-dimensional structures self-assembled from DNA bricks,” Science 338(6111): 1177-1183 (2012); Stone et al., “Molecular Mechanics of DNA Bricks: In Situ Structure, Mechanical Properties and Ionic Conductivity,” New J. Phys. 18:055012 (2016); Green et al., “Autonomous Dynamic Control of DNA Nanostructure Self-Assembly,” Nature Chem. 11:510-520 (2019); Gradišar et al., “Self-Assembled Bionanostructures: Proteins Following the Lead of DNA Nanostructures,” J. Nanobiotechnology 12:4 (2014); Huang et al., “Sefl-Assembled DNA Nanostructures-Based Nanocarriers Enabled Functional Nucleic Acids Delivery,” ACS Appl. Bio. Mater. 3:2779-2795 (2020); Bujold et al., “DNA Nanostructures at the Interface with Biology,” Chem. 4:495-521 (2018), which are hereby incorporated by reference in their entirety), DNA wireframe (Rogers et al., “Using DNA to Program the Self-Assembly of Colloidal Nanoparticles and Microparticles,” Nature Reviews Materials 1:16008 (2016); Zhang et al., “DNA Self-Assembly Scaled Up,” Nature 552:34-35 (2017); Green et al., “Autonomous Dynamic Control of DNA Nanostructure Self-Assembly,” Nature Chemistry 11:510-520 (2019); Bujold et al., “DNA Nanostructures at the Interface with Biology,” Chem. 4:495-521 (2018), which are hereby incorporated by reference in their entirety), DNA tile (Ke et al., “Three-Dimensional Structures Self-Assembled from DNA Bricks,” Science 338(6111): 1177-1183 (2012); Rothermund et al., “Design and Characterization of Programmable DNA Nanotubes,” J. Am. Chem. Soc. 126:16344-16352 (2004); Green et al., “Autonomous Dynamic Control of DNA Nanostructure Self-Assembly,” Nature Chem. 11:510-520 (2019); Huang et al., “Sefl-Assembled DNA Nanostructures-Based Nanocarriers Enabled Functional Nucleic Acids Delivery,” ACS Appl. Bio. Mater. 3:2779-2795 (2020); Bujold et al., “DNA Nanostructures at the Interface with Biology,” Chem. 4:495-521 (2018), which are hereby incorporated by reference in their entirety), and DNA hydrogel (Huang et al., “Sefl-Assembled DNA Nanostructures-Based Nanocarriers Enabled Functional Nucleic Acids Delivery,” ACS Appl. Bio. Mater. 3:2779-2795 (2020); Khajouei et al., “DNA Hydrogel-Empowered Biosensing,” Advances in Colloid and Interface Science 275:102060 (2020); and Li et al., “Polymeric DNA Hydrogel: Design, Synthesis, and Applications,” Progress in Polymer Science 98:101163 (2019), which are hereby incorporated by reference in their entirety).

Advantages of DNA nanoparticles include, but are not limited to, high programmability, biocompatibility, vast chemical malleability, and low cost/high yield production (Chi et al., “DNA Nanostructure as an Efficient Drug Delivery Platform for Immunotherapy,” Front. Pharmacol. 10:1585 (2020); Dey et al., “DNA Origami,” Nature Reviews Methods Primers 1:12 (2021); and Jiang et al., “DNA Origami as Carrier for Circumvention of Drug Resistance,” J. Am. Chem. Soc. 134(32): 13396-13403 (2012), which are hereby incorporated by reference in their entirety). In some embodiments, the present disclosure uses DNA origami method to construct self-assembly DNA nanoparticles, however the scope of the disclosure includes all existing and emerging DNA-self-assembly methods and strategies.

As described supra, in the DNA origami method, DNA nanoparticles are self-assembled by mixing a long single-stranded DNA (ssDNA) molecule called the “scaffold” with short oligomers known as “staples” usually in the presence of a stabilizing concentration of magnesium ions (Mg++) (Chi et al., “DNA Nanostructure as an Efficient Drug Delivery Platform for Immunotherapy,” Front. Pharmacol. 10:1585 (2020); Dey et al., “DNA Origami,” Nature Reviews Methods Primers 1:12 (2021); and Berengut et al., “Self-Limiting Polymerization of DNA Origami Subunits with Strain Accumulation,” ACS Nano 14(12): 17428-17441 (2020), which are hereby incorporated by reference in their entirety).

The surface of DNA nanoparticles made this way can be coated with various chemical components for enhancing in vivo stability, delivery, target recognition, and function. Intercellular delivery of molecules using DNA nanoparticles has been demonstrated in vivo with Green Fluorescent Protein (GFP), thrombin, and doxorubicin, with promising results (Jiang et al., “DNA Origami as Carrier for Circumvention of Drug Resistance,” J. Am. Chem. Soc./34(32): 13396-13403 (2012); Burns et al., “DNA Origami Inside-Out Viruses,” ACS Synth. Biol. 7:767-773 (2018); and Li et al., “A DNA Nanorobot Functions as a Cancer Therapeutic in Response to a Molecular Trigger in vivo,” Nature Biotechnol. 36:258-264 (2018), which are hereby incorporated by reference in their entirety). Conventional DNA origami methods usually employ M13mp18 ssDNA as the scaffold due to its commercial availability in single-stranded form (Dey et al., “DNA Origami,” Nature Reviews Methods Primers 1:12 (2021) and Bush et al., “Synthesis of DNA Origami Scaffolds: Current and Emerging Strategies,” Molecules 25:3386 (2020), which are hereby incorporated by reference in their entirety).

It is important to note that to date, DNA origami nanoparticle mediated in vivo delivery has employed the scaffold as a relatively inert architectural or mechanical component of the system. Thus, the delivery method has been constrained to encapsulation or loading of molecules inside, or onto DNA nanoparticles (Jiang et al., “DNA Origami as Carrier for Circumvention of Drug Resistance,” J. Am. Chem. Soc. 134(32): 13396-13403 (2012); Burns et al., “DNA Origami Inside-Out Viruses,” ACS Synth. Biol. 7:767-773 (2018); and Li et al., “A DNA Nanorobot Functions as a Cancer Therapeutic in Response to a Molecular Trigger in vivo,” Nature Biotechnol. 36:258-264 (2018), which are hereby incorporated by reference in their entirety). In stark contrast to this strategy, the present disclosure describes construction of nucleic acid nanoparticles whose scaffold is readily transcribable in vitro and expressed in vivo and ex vivo.

Accordingly, a first aspect of the present disclosure relates to a nucleic acid nanoparticle comprising a single stranded nucleic acid scaffold encoding one or more RNA molecules and/or proteins of interest and a plurality of single stranded nucleic acid oligomers, where each oligomer is complementary to at least a portion of the single stranded nucleic acid scaffold.

The single stranded nucleic acid scaffold may be a DNA molecule, an RNA molecule, or comprise a mixture of DNA and RNA nucleotides.

In some embodiments, the single stranded nucleic acid scaffold encodes one or more RNA molecules. Suitable RNA molecules include, without limitation, a messenger RNA (mRNA), an aptamer, a small interfering RNA (siRNA), a short hairpin RNA (shRNA), a microRNA (miRNA), a non-coding RNA (ncRNA), a guide RNA (gRNA), and combinations thereof.

In some embodiments, the single stranded nucleic acid scaffold encodes one or more protein(s) of interest.

In some embodiments, the single stranded nucleic acid scaffold encodes a combination of RNA molecule(s) and proteins(s) of interest.

The one or more proteins of interest may comprise one or more antigens. Suitable antigens include, without limitation, an antigen derived from a pathogenic microorganism; a tumor-associated antigen; and an allergen. Antigens derived from a pathogenic microorganism include antigens derived from a virus, a bacterium, a fungus, a protozoan, or a helminth. The antigen may be any target epitope, molecule, or molecular complex against which elicitation or enhancement of immunogenicity in a subject is desired.

A suitable antigen can be any type of antigen known in the art and include, e.g., full-length antigens, truncated antigens, mutated antigens, and inactivated or combined forms from a single pathogen or different pathogen(s) or tumor-associated cancer antigens. Suitable antigens include, e.g., proteins, recombinant proteins, recombinant fusion proteins, proteins and peptides conjugated to toll-like receptor (TLR) agonists, proteins and peptides conjugated to bacterial toxins, proteins and peptides conjugated to antibodies, proteins and peptides conjugated to cytokines and chemokines, glycoproteins, glycolipoproteins and derivatives thereof.

In some embodiments, the single stranded nucleic acid scaffold encodes a single RNA and/or protein of interest.

In some embodiments, the single stranded nucleic acid scaffold encodes at least two, at least three, at least four, at least five, at least six, at least seven, at least eight, at least nine, at least ten, or more RNA and/or proteins of interest. Where a nucleic acid scaffold according to the present disclosure encodes more than one antigen, the more than one antigen can be from the same pathogenic organism, or from the same cancer cell. Where a nucleic acid scaffold according to the present disclosure includes more than one antigen, the more than one antigen can be from two or more different pathogenic organisms, or from two or more different cancer cells or two or more different types of cancers.

In some embodiments, the nucleic acid scaffold according to the present disclosure encodes a gene-editing system. In some embodiments, the nucleic acid scaffold according to the present disclosure encodes one or more components of a gene-editing system.

The gene-editing system may be a nuclease-based gene-editing system. As used herein, the term “nuclease-based gene editing system” refers to a system comprising a nuclease or a derivative thereof that can be recruited to a target sequence in the genome. Thus, in some embodiments, the gene-editing system may be selected from the group consisting of a Clustered Regularly Interspaced Short Palindromic Repeat/Clustered Regularly Interspaced Short Palindromic Repeat associated (CRISPR/Cas) system, a zinc finger nuclease (“ZFNs”), or a transcription activator-like effector nucleases (“TALEN”).

In some embodiments, the nuclease-based gene editing system is a CRISPR/Cas system. The CRISPR/Cas system may comprise a Cas protein or a nucleic acid molecule encoding the Cas protein and a guide RNA comprising a nucleotide sequence that is complementary to a portion of a target DNA sequence.

As described herein, Cas proteins form a ribonucleoprotein complex with a guide RNA, which guides the Cas protein to a target DNA sequence. Suitable Cas proteins include Cas nucleases (i.e., Cas proteins capable of introducing a double strand break at a target nucleic acid sequence), Cas nickases (i.e., Cas protein derivatives capable of introducing a single strand break at a target nucleic acid sequence), and nuclease dead Cas (dCas) proteins (i.e., Cas protein derivatives that do not have any nuclease activity).

In some embodiments, the Cas protein is a Cas9 protein. As used herein, the term “Cas9 protein” or “Cas9” includes any of the recombinant or naturally-occurring forms of the CRISPR-associated protein 9 (Cas9) or variants or homologs thereof. In some embodiments, the Cas9 protein is substantially identical to the protein identified by the UniProt reference number Q99ZW2, G3ECR1, J7RUA5, AOQ5Y3, or J3F2B0 (which are hereby incorporated by reference in their entirety) or a variant or homolog having substantial identity thereto.

In some embodiments, the Cas protein is a Cas12a protein. As used herein, the term “Cas12a protein” or “Cas12a” includes any of the recombinant or naturally-occurring forms of the CRISPR-associated protein 12 (Cas12a) or variants or homologs thereof. In some embodiments, the Cas 12a protein is substantially identical to the protein identified by the UniProt reference number AOQ7Q2, U2UMQ6, AOA7C6JPC1, A0A7C9HOZ9, or A0A7JOAY55 (which are hereby incorporated by reference in their entirety) or a variant or homolog having substantial identity thereto.

In some embodiments, the Cas protein is a Cas 12b protein. As used herein, the term “Cas12b protein” or “Cas12b” includes any of the recombinant or naturally-occurring forms of the CRISPR-associated protein 12 (Cas12b) or variants or homologs thereof. In some embodiments, the Cas12b protein is substantially identical to the protein identified by the UniProt reference number TOD7A2, A0A613SPI6, A0A617FUC4, A0A6N9TP17, A0A6MIUF64, A0A7Y8V748, A0A7X7KIS4, A0A7X8X2U5, or A0A7X8UMW7 (which are hereby incorporated by reference in their entirety) or a variant or homolog having substantial identity thereto.

As used herein, the term “guide RNA” or “gRNA” refers to a ribonucleotide sequence capable of binding a nucleoprotein, thereby forming a ribonucleoprotein complex. In accordance with the methods and systems of the present disclosure, the guide RNA comprises (i) a DNA-targeting sequence that is complementary to a target nucleic acid sequence and (ii) a binding sequence for the Cas protein (e.g., Cas9 nuclease, Cas9 nickase, dCas9, Cas12a nuclease, Cas12a nickase, or dCas12a).

In some embodiments, the single stranded nucleic acid scaffold encodes two RNA molecules (e.g., joined together via hybridization at the binding sequence). In accordance with such embodiments, the guide RNA comprises a crispr RNA (crRNA), a 17-20 nucleotide sequence complementary to the target DNA, and a tracr RNA, which serves as a binding scaffold for the Cas nuclease.

In some embodiments, the single stranded nucleic acid scaffold encodes a single guide RNA molecule (single RNA nucleic acid), which may include a “single-guide RNA” or “sgRNA”. The sgRNA may be 15, 16, 17, 18, 19, 20, 21, 22, 23, 24, 25, 26, 27, 28, 29, 30, 40, 50, 60, 70, 80, 90, 100, 110, 120, 130, 140, 150, or more nucleic acid residues in length. In some embodiments, the sgRNA is from 20 to 120 nucleic acid residues in length, 20 to 120 nucleic acid residues in length, or 20 to 100 nucleic acid residues in length.

The Examples of the present disclosure demonstrate that a gene-bearing scaffold in a nucleic acid nanoparticle can be transcribed in vitro and in cell culture. Thus, it is believed that similarly structured nucleic acid nanoparticles will be transcribed in vivo, resulting in the production of the encoded gene products.

Also, it is believed that the inclusion of cationic lipid and targeting peptide sequences coupled to the gene-bearing nucleic acid nanoparticles may enhance structural integrity and functionality in vivo and facilitate delivery and intracellular trafficking to the nucleus where the camouflaged genes in the scaffold will be transcribed and the resultant mRNA exported and translated. Thus, in some embodiments, the single stranded nucleic acid scaffold and/or the plurality of single stranded nucleic acid oligomers comprise a cationic lipid moiety.

As used herein, the term “lipid” refers to a group of organic compounds that include, but are not limited to, esters of fatty acids and are characterized by being insoluble in water, but soluble in many organic solvents. The term “cationic lipid” refers to any of a number of lipid species that carry a net positive charge at a selected pH, such as physiological pH. Such lipids include, but are not limited to: 1,2-DiLinoleyloxy-N,N-dimethylaminopropane (DLinDMA) and 1,2-Dilinolenyloxy-N,N-dimethylaminopropane (DLenDMA); N,N-diolcyl-N,N-dimethylammonium chloride (DODAC); N-(2,3-diolcyloxy)propyl)-N,N,N-trimethylammonium chloride (DOTMA); N,N-distearyl-N,N-dimethylammonium bromide (DDAB); N-(2,3-diolcoyloxy)propyl)-N,N,N-trimethylammonium chloride (DOTAP); 3—(N—(N′,N′-dimethylaminocthanc)-carbamoyl)cholesterol (DC-Chol); N-(1,2-dimyristyloxyprop-3-yl)-N,N-dimethyl-N-hydroxyethyl ammonium bromide (DMRIE); and derivatives thereof. The following lipids are cationic and have a positive charge at below physiological pH: DODAP, DODMA, DMDMA and the like.

In some embodiments, the single stranded nucleic acid scaffold and/or the plurality of single stranded nucleic acid oligomers comprise a targeting peptide sequence, e.g., a cell penetrating peptide (CPP) or a nuclear localization signal (NLS).

As used herein, the term “cell penetrating peptide” or “CPP” refers to a peptide which can transport cargo (e.g., the nucleic acid nanoparticle according to the present disclosure) across the plasma membrane into a cell. Cell penetrating peptides are generally short cationic and/or amphipathic peptide sequences, often between 20 and 50 residues in length, characterized by an ability to translocate across the membrane systems of mammalian cells, localize in one or more intracellular compartments, and mediate intracellular delivery of a cargo molecule (e.g., the nucleic acid nanoparticle according to the present disclosure). Suitable cell penetrating peptides are well known in the art and include, without limitation, HIV-1 Trans-Activator of Transcription (Tat) Peptide, Syn-B1, Syn B-3, Poly-L-arginine, penetratin (43-58), amphipathic model peptide, transportin, SBP, and FBP (see, e.g., Sharma et al., “The Role of Cell-Penetrating Peptide and Transferrin on Enhanced Delivery of Drug to Brain,” Int. J. Mol. Sci. 17(6):806 (2016), which is hereby incorporated by reference in its entirety).

TABLE 1
Exemplary Cell Penetrating Peptides

| Cell Penetrating Peptide | Sequence | SEQ ID NO: |
|---|---|---|
| HIV Tat peptide (48-60) | GRKKRRQRRRPPQ | 1 |
| Syn-B1 | RGGRLSYSRRRFSTSTGR | 2 |
| Syn-B3 | RRLSYSRRRF | 3 |
| Poly-L-arginine | RRRRRRR | 4 |
| Poly-L-arginine | RRRRRRRRR | 5 |
| penetratin (43-58) | RQIKIWFQNRRMKWKK | 6 |
| amphipathic model peptide | KLALKLALKALKAALKLA | 7 |
| transportin | GWTLNSAGYLLKINLKALAALAKKIL | 8 |
| SBP | MGLGLHLLVLAAALQGAWSQPKKKRKV | 9 |
| FBP | GALFLGWLGAAGSTMGAWSQPKKKRKV | 10 |

As used herein, the term “nuclear localization signal” or “NLS” refers to an amino acid sequence known to, in vivo, direct a protein disposed in the cytoplasm of a cell across the nuclear membrane and into the nucleus of the cell. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 35, 30, 25, 15, 12, 10, 8, 7, 6, 5, or 4 amino acids, but will in some embodiments comprise at least a four to eight amino acid sequence known to function as a nuclear localization signal.

Suitable nuclear localization signals are well known in the art and include, without limitation, those identified in Table 2 below (see, e.g., Lu et al., “Types of Nuclear Localization Signals and Mechanisms of Protein Import into the Nucleus,” Cell Communication and Signaling 19:60 (2021), which is hereby incorporated by reference in its entirety).

TABLE 2
Exemplary Nuclear Localization Signals

| Cell Penetrating | | |
|---|---|---|
| Peptide | Sequence | SEQ ID NO: |
| SV40 Large T- | PKKKRKV | 11 |
| antigen | | |
| VACM-1/CUL5 | PKLKRQ | 12 |
| CXCR4 | RPRK | 13 |
| VP1 | RRARRPRG | 14 |
| 53BP1 | GKRKLITSEEERSPAKRGRKS | 15 |
| ING4 | KGKKGRTQKEKKAARARSKGKN | 16 |
| IER5 | RKRCAAGVGGGPAGCPAPGSTPLKKPRR | 17 |
| ERK5 | RKPVTAQERQREREEKRRRRQERAKEREKRRQERER | 18 |
| Hrp1 | RSGGNHRRNGRGGRGGYNRRNNGYHPY | 19 |
| UL79 | TLLLRETMNNLGVSDHAVLSRKTPQPY | 20 |
| EWS | PGKMDKGEHRQERRDRPY | 21 |
| parathyroid | GKKKKGKPGKRREQRKKKRRT | 22 |
| hormone-related | | |
| protein (PtHrP) | | |
| Pho4 | SANKVTKNKSNSSPYLNKRKGKPGPDS | 23 |
| rpL23a | VHSHKKKKIPTSPTFTTPKTLTLRRQPKYPRKSAPRRNKLDHY | 24 |
| MSX1 | RKHKTNRKPR | 25 |
| MSX1 | NRRAKAKR | 26 |
| NLS-RARa | RNKKKK | 27 |
| NLS-RARa | RKVIK | 28 |
| Bipartite | KRPAAIKKAGQAKKKK | 29 |
| nucleoplasmin | | |
| NLS (155-170) | | |

In some embodiments, the nuclear localization signal is selected from the group consisting of SV40 Large T-antigen, VACM-1/CUL5, CXCR4, VP1, 53BP1, ING4, IER5, ERK5, Hrp1, UL79, EWS, PtHrP, Pho4, rpL23a, MSX1, NLS-RARa, bipartite nucleoplasmin NLS, and derivatives thereof.

In some embodiments, the single stranded nucleic acid scaffold and/or the plurality of single stranded nucleic acid oligomers comprise one or more biodegradable cationic polymers. Suitable biodegradable cationic polymers include, without limitation, polyethylene glycerol (PEG)-poly-lysine copolymers (see, e.g., Bikram et al., “Biodegradable Poly(ethylene glycol)-co-poly(l-lysine)-g-histidine Multiblock Copolymers for Nonviral Gene Delivery,” Macromolecules 37(5): 1903-1916 (2004), which is hereby incorporated by reference in its entirety).

In some embodiments, the single stranded nucleic acid scaffold and/or the plurality of single stranded nucleic acid oligomers comprise one or more peptoids. As used herein, the term “peptoid” refers to an N-substituted glycine polymer in which the side chains are appended to the backbone nitrogen. In some embodiments, the peptoid is a lipid peptoid.

In some embodiments, the single stranded nucleic acid scaffold is not modified. In accordance with such embodiments, the single stranded nucleic acid scaffold does not comprise a cationic lipid moiety, a targeting peptide sequence, a biodegradable cationic polymer, and/or a peptoid.

In some embodiments, when the single stranded nucleic acid scaffold is not modified, at least a portion of the plurality of single stranded nucleic acid oligomers is modified. In accordance with such embodiments, at least a portion of the plurality of single stranded nucleic acid oligomers comprise a cationic lipid moiety, a targeting peptide sequence, a biodegradable cationic polymer, and/or a peptoid. In some embodiments, each of the plurality of single stranded nucleic acid oligomers comprise a cationic lipid moiety, a targeting peptide sequence, a biodegradable cationic polymer, and/or a peptoid.

In some embodiments, the single stranded nucleic acid scaffold further comprises a promoter and/or an enhancer.

As used herein, the term “promoter” refers to a nucleotide sequence that permits binding of RNA polymerase and directs the transcription of a gene or a nucleic acid sequence. The promoter may be a viral, a prokaryotic, or a eukaryotic promoter sequence.

The term “enhancer” refers to a segment of DNA which contains sequences capable of providing enhanced transcription and in some instances can function independent of their orientation relative to another control sequence. Enhancers are cis-acting DNA sequences that can function cooperatively or additively with promoters and/or other enhancer elements. The term “promoter/enhancer” or “promoter-inclusive regulatory element” refers to a segment of DNA which contains sequences capable of providing both promoter and enhancer functions. The enhancer may be a viral, a prokaryotic, or a eukaryotic enhancer sequence.

In some embodiments, the single stranded nucleic acid scaffold further comprises a transcriptional terminator. As used herein, the term “transcriptional terminator” refers to a DNA sequence which promotes the formation of a 3′ end of an RNA transcript. As used herein the term “transcriptional terminator” refers to viral, a prokaryotic, or a eukaryotic transcriptional terminator sequences (e.g., polyadenylation signal sequences).

### Vaccine Compositions

Regulation of gene expression underlies both normal and aberrant cellular function. Thus, understanding and regulating gene expression is important for normal cell function and a centerpiece in the emergence of a vast array of syndromes and diseases including, for example, diabetes and cancer. There are many biological mechanisms in place to regulate gene activity ranging from heterochromatic inhibition of gene access to modulation of RNA transcript lifetime and protein product activity and accessibility. Further, there are emerging therapeutic tools, such as the ever-expanding array of gene editors, for regulating gene expression to treat and cure disease at both the gene and the epigenome levels. In some embodiments, the present disclosure utilizes the programmable nature of DNA via the method of DNA origami (Rothemund, P. W. K., “Folding DNA to Create Nanoscale Shapes and Patterns,” Nature 440(7082):297-302 (2006), which is hereby incorporated by reference in its entirety) and related methods to “fold” nucleic acid molecules into architectures that attenuate/regulate RNA polymerase activity and thereby gene expression in a controllable fashion. Gene activity can be regulated by, among other things, adjusting the compactness of the gene(s) and thereby delaying the onset of the expression, or regulating the initiation of gene expression itself through approaches such as burying the promoter in a controllably accessible portion of the DNA origami architecture.

In some embodiments, the present disclosure provides a nucleic acid nanoparticle constructed such that the “scaffold” is comprised of a long gene-bearing ssDNA polynucleotide and is folded in a variety of configurations (i.e., is programmably sculpted) to hide or reveal biologically relevant regions of the gene-bearing scaffold to varying degrees.

In some embodiments, the nucleic acid nanoparticle may have a honeycomb structure, a cuboid structure, a columnar structure, a cylindrical structure, a sheet structure, a hexagonal lattice structure, or a combination of any such structures.

In some embodiments, the nucleic acid nanoparticle comprises a linear promoter and/or enhancer.

In some embodiments, the nucleic acid nanoparticle comprises a folded promoter and/or enhancer.

It has been demonstrated that DNA origami may be utilized to deliver genetic material to living cells. In particular, a piece of genetic material was folded by DNA origami and co-delivered with the machinery for gene editing by the CRISPR/Cas9 mechanism resulting in the successful insertion of a genetic element at a specific genomic location (Lin-Shiao et al., “CRISPR-Cas9-Mediated Nuclear Transport and Genomic Integration of Nanostructured Genes in Human Primary Cells,” Nucleic Acids Res. 50(3): 1256-1268 (2022), which is hereby incorporated by reference in its entirety). Thus, the foundation has been laid for using programmable DNA nanostructures as delivery mechanisms for genetic materials and the present disclosure expands upon that platform by demonstrating an inventive improvement involving the programming of these nanostructures to control the degree of gene expression. This capability could prove crucial in the expression of disease-rectifying genes that are delivered in vivo as therapeutic agents and in some cases as complete cures.

Accordingly, another aspect of the present disclosure relates to a pharmaceutical composition comprising a nucleic acid nanoparticle as described herein and a pharmaceutically acceptable carrier.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). In some embodiments, the term “pharmaceutically acceptable” means approved by a regulatory agency or listed in the U.S. Pharmacopeia or other generally recognized pharmacopeia for use in mammals, and more particularly in humans.

A “pharmaceutically acceptable carrier” refers to a diluent, adjuvant, excipient, or vehicle with which the compound is administered. Such pharmaceutically acceptable carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions may be employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutically acceptable carriers are described in “Remington's Pharmaceutical Sciences” by E. W. Martin.

Pharmaceutical compositions comprising the nucleic acid nanoparticles described herein may comprise buffers such as neutral buffered saline, phosphate buffered saline, and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

### Nanoparticle Vaccines

Creating a DNA nanoparticle vaccine usually beings with the production of the gene-bearing ssDNA scaffold. There are several methods for producing relatively large and pure quantities of ssDNA, each with its virtues and liabilities. Several of these are mentioned here as examples and are not intended to limit the scope of the disclosure.

ssDNA Scaffold Production

Asymmetric PCR (aPCR) and Rolling Circle Amplification (RCA) can generate large quantities of ssDNA. Thus, these methods can be employed to produce custom ssDNA scaffolds comprised of the antisense (template) strand of an antigen gene of interest and all other genetic elements required (e.g., promoter, enhancer elements) to ensure effective and faithful transcription and subsequent translation of the gene thus produced. With minor adjustments of the parameters, the scaffold synthesis can be scaled up to industrial levels. In the case of aPCR, Veneziano et al. and Sanchez et al. provided guidance for optimizing key aPCR parameters, including polymerase choice and primer design (Veneziano et al., “In vitro Synthesis of Gene-Length Single-Stranded DNA,” Scientific Reports 8:6548 (2018) and Sanchez et al., “Linear-After-The-Exponential (LATE)-PCR: An Advanced Method of Asymmetric PCR and its uses in Quantitative Real-Time Analysis,” PNAS 101(7): 1933-1938 (2004), which are hereby incorporated by reference in their entirety). Further, Tolnai et al. developed a simple modification to enhance aPCR efficiency (Tolnai et al., “A Simple Modification Increases Specificity and Efficiency of Asymmetric PCR,” Analytica Chimica Acta 1047:225-230 (2018), which is hereby incorporated by reference in its entirety). With regard to RCA, Ducani et al. have developed this method for efficient ssDNA synthesis including the use of single stranded binding protein (SSB) and a restriction site hairpin loop for production of large quantities of ssDNA monomers (Ducani et al., “Rolling Circle Replication Requires Single-Stranded DNA Binding Protein to Avoid Termination and Product of Double-Stranded DNA,” Nucleic Acids Res. 42(16): 10596-604 (2014) and Ducani et al., “Enzymatic Production of Monoclonal Stoichiometric Single-Stranded DNA Oligonucleotides,” Nat. Methods 10(7):647-652 (2013), which are hereby incorporated by reference in their entirety).

Additional methods to produce ssDNA scaffolds include, without limitation, thermal/chemical denaturation and strand-specific endonuclease digestion.

### DNA Nanoparticle Design

A number of strategies and methods for creating self-assembling DNA nanoparticles exist, many of which are variants of a simple one-pot, heat and cool reaction (Rothermund P W K, “Folding DNA to Create Nanoscale Shapes and Patterns,” Nature 440(7082):297-302 (2006) and Practorius et al., “Biotechnological Mass Production of DNA Origami,” Nature 552(7683):84-87 (2017), which are hereby incorporated by reference in their entirety). The simplicity of this process illustrates a powerful feature of the present disclosure: DNA (or RNA) contains embedded in its primary sequence an engineering code that mediates the self-assembly process in a single reaction. Thus, enormous variability and combinatorial opportunities are made possible by the inventive work described herein.

There are a variety of approaches for the design and production of DNA nanoparticles. Although the present disclosure uses the method of DNA origami by way of example that choice is not meant to limit the scope of the present disclosure. The design of DNA origami nanoparticles is readily accomplished by hand or through the use of software including, but not limited to, caDNAno. Chemically synthesized oligonucleotide staples, customizable a variety of ways, are commercially available from numerous sources. Alternatively, for industrial level production, staple sets can be cloned into either phagemid or plasmid vectors for in vitro production by RCA or phagemid vectors for in vivo production in bacteria (Ducani et al., “Enzymatic Production of Monoclonal Stoichiometric Single-Stranded DNA Oligonucleotides,” Nat. Methods 10(7):647-652 (2013) and Nafisi et al., “Construction of a novel phagemid to produce custom DNA origami scaffolds,” Synthetic Biology, 3(1): ysy015 (2018), which are hereby incorporated by reference in their entirety).

A vast spectrum of modifications is possible with DNA nanoparticles due to the extremely malleable chemical nature of nucleic acids. For example, they may be coated with cell penetrating peptides (CPP), nuclear localization signal (NLS), lipids, hydrophobic patches, and a plethora of other molecular and chemical modifications to enhance functionality. These modifications can be both covalent and non-covalent for optimal and targeted cellular and intercellular delivery.

In one example not intended to limit the scope of the invention, HIV-Tat1, a well-known CPP for efficient delivery of molecules into the cell nucleus, may be tethered to DNA nanoparticles (Bolhassani et al., “In vitro and In vivo Delivery of Therapeutic Proteins Using Cell Penetrating Peptides,” Peptides 87:50-63 (2017) and Smith et al., “Structural Basis for Importin-α Binding of the Human Immunodeficiency Virus Tat,” Scientific Reports 7:1650 (2017), which are hereby incorporated by reference in their entirety).

Any one of numerous methods may be used to covalently or non-covalently couple peptides to oligonucleotide staples (Sutherland et al., “Utility of Formaldehyde Cross-Linking and Mass Spectrometry in the Study of Protein-Protein Interactions,” J. Mass Spectrom. 43(6): 699-715 (2008) and Mikkilä et al., “Virus-Encapsulated DNA Origami Nanostructures for Cellular Delivery,” Nano Lett. 14(4):2196-2200 (2014), which are hereby incorporated by reference in their entirety). As described herein, multiple modifications can be employed in parallel to further enhance functionality and stability in vivo. Coupled with CPP and NLS, various hydrophobic patches may be employed to enhance transfection efficiencies, structural integrity, and protection against nucleases in vivo. A non-limiting example is to couple N-(2-aminoethyl)glycine and neutral N-2-(2-(2-methoxyethoxy)ethoxy)ethlyglycine or lipid glycine, alternatively, through solid phase peptoids synthesis to create “brush” type peptoids with one neutral and one positively charged terminus. Brush-type peptoids are known to electrostatically bind to the negatively charged DNA backbone of DNA nanoparticle surfaces, thereby coating them (Wang et al., “DNA Origami Protection and Molecular Interfacing through Engineered Sequence-Defined Peptoids,” PNAS 117(12):6339-6348 (2020), which is hereby incorporated by reference in its entirety). Another non-limiting example is PEG poly-lysine copolymer. Similar to “brush” type, PEG poly-lysine copolymer involves a positively charged poly-lysine domain and neutral PEG domain. The positively charged poly-lysine domain binds electrostatically to the negatively charged DNA backbone (Agarwal et al., “Block Copolymer Micellization as Protection Strategy for DNA Origami,” Angew. Chem. Int. Ed. Engl. 56(20):5460-5464 (2017) and Ponnuswamy et al., “Oligolysine-Based Coating Protects DNA Nanostructures from Low-Salt Denaturation and Nuclease Degradation,” Nature Comm. 8:15654 (2017), which are hereby incorporated by reference in their entirety). This interaction not only masks DNA nanoparticles, providing protection from nucleases, but also enhances structural integrity in low magnesium environments (i.e., physiological conditions) by neutralizing negative charges on the DNA backbone that can cause repulsive destabilization of DNA nanoparticles (Wang et al., “DNA Origami Protection and Molecular Interfacing through Engineered Sequence-Defined Peptoids,” PNAS 117(12):6339-6348 (2020), which is hereby incorporated by reference in its entirety).

### Advantages of the Nucleic Acid Nanoparticle and Pharmaceutical Compositions According to the Present Disclosure

Targeting. Once delivered in vivo, a targeting peptide such as, but not limited to, HIV1-Tat will direct the synthetic virus vaccine into the cell nucleus.

Gene-Expression and Antigen Production. Once inside the nuclei of the target cell(s) or tissue(s) the structured and camouflaged synthetic virus reveals the scaffold which serves as the template for subsequent transcription and subsequent translation resulting in antigen. The process of transcription, copying the gene and producing a messenger RNA containing the encoded gene, involves separation of the two strands of the DNA double helix. This process liberates the staples that camouflage and retain the nanoparticle in the desired configuration. The full gene is thereby revealed and the complete mRNA produced.

The Nucleic Acid Nanoparticle is a Vaccine. In some embodiments, the nucleic acid nanoparticles produced constitute a key element of a vaccine. A key distinction between current DNA-based vaccines and the synthetic virus vaccine embodiments described herein is the lack of reliance on viral vectors, thereby mitigating the aforementioned caveats (reiterated below) associated with those carriers. The defining feature of the synthetic virus vaccine is that the DNA nanoparticle, in particular the scaffold element, serves as both the carrier and the active genetic element that produces the vaccine antigen. Independence from viral vectors alleviates several limitations of current DNA-based vaccines. In some embodiments, the benefits include the following:

Benefit 1. The size of the antigen gene is no longer constrained by loading limits of a viral vector (Mout et al., “In Vivo Delivery of CRISPR/Cas9 for Therapeutic Gene Editing: Progress and Challenges,” Bioconjugate Chem. 28:880-884 (2017), which is hereby incorporated by reference in its entirety).

Benefit 2. Transfection efficiency of synthetic virus vaccine remains unaffected by preexisting immunity that can compromise the efficacy of virally delivered vaccines.

Benefit 3. The use of highly chemically malleable DNA as the nanoparticle and the active antigen gene allows implementation of a vast trove of modifications to fine tune performance of the system.

Benefit 4. The greater stability of DNA relative to RNA portends enhanced vaccine stability for long term storage and global delivery. This does not exclude the use of RNA nanoparticles in practicing the invention, however.

Benefit 5. The production of synthetic DNA is highly cost effective.

Benefit 6. The nature of the platform allows for rapid and vast combinatorial assembly of vaccine variants to optimize efficacy against an ever-changing viral landscape.

Another aspect of the present disclosure relates to a method of eliciting an immune response in a subject. This method involves administering a nucleic acid nanoparticle or a pharmaceutical composition described herein to a subject, where said administering elicits an immune response in the subject.

Suitable nucleic acid nanoparticles and vaccine compositions comprising nucleic acid nanoparticles according to the present disclosure are described in detail supra.

Suitable subjects that can be treated in accordance with the methods disclosed herein include, without limitation mammals, such as humans, non-human primates, and veterinary animals (e.g., cats, dogs, cows, horses, sheep, pigs, etc.) and experimental animal models. In some embodiments, the subject is a human.

In accordance with this and all other aspects of the present disclosure, the term “immune response” refers to the development in a subject of a humoral and/or a cellular immune response to, e.g., an antigen encoded by the nucleic acid nanoparticle or a pharmaceutical composition according to the present disclosure. A “humoral immune response” refers to an immune response mediated by antibody molecules, while a “cellular immune response” is one mediated by T-lymphocytes and/or other white blood cells. An immune response may include one or more of the following effects: the production of antibodies by B-cells and/or the activation of suppressor, cytotoxic, or helper T-cells and/or T-cells directed specifically to an antigen or antigens present in the composition or vaccine of interest. These responses may serve to neutralize infectivity and/or mediate antibody-complement, or antibody-dependent cell cytotoxicity (ADCC) to provide protection to an immunized host. Such responses can be determined using standard immunoassays and neutralization assays well known in the art.

The nucleic acid nanoparticle or a pharmaceutical composition according to the present disclosure may be administered to the subject using methods known in the art including parenteral, topical, intravenous, oral, subcutaneous, intraperitoneal, intranasal, or intramuscular means. In some embodiments, the nucleic acid nanoparticles or pharmaceutical compositions according to the present disclosure are formulated for subcutaneous administration. In some embodiments, the nucleic acid nanoparticles or pharmaceutical compositions according to the present disclosure are formulated for intramuscular injection. In some embodiments, this type of injection is performed in the arm or leg muscles. Intravenous injections as well as intraperitoneal injections, intraarterial, intracranial, or intradermal injections may also be effective in generating an immune response.

In some embodiments, the nucleic acid nanoparticles or pharmaceutical compositions according to the present disclosure are formulated for parenteral administration. Solutions or suspensions of the nucleic acid nanoparticles or pharmaceutical compositions can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and/or sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the nucleic acid nanoparticles or pharmaceutical compositions of the present disclosure systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents.

Intraperitoneal or intrathecal administration of the nucleic acid nanoparticles or pharmaceutical compositions of the present disclosure can also be achieved using infusion pump devices such as those described by Medtronic, Northridge, CA. Such devices allow continuous infusion of desired compounds avoiding multiple injections and multiple manipulations.

In addition to the formulations described previously, the nucleic acid nanoparticles or pharmaceutical compositions of the present disclosure may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Detection of an effective immune response may be determined by a number of assays known in the art. For example, a cell-mediated immunological response can be detected using methods that include lymphoproliferation (lymphocyte activation) assays, CTL cytotoxicity assays, or by assaying for T-lymphocytes specific for the antigen in a sensitized subject. Such assays are well known in the art.

The presence of a humoral immunological response can be determined and monitored by testing a biological sample (e.g., blood, plasma, serum, urine, saliva feces, CSF or lymph fluid) from the mammal for the presence of antibodies directed to the immunogenic component of the administered polymerized product. Methods for detecting antibodies in a biological sample are well known in the art, e.g., ELISA, Dot blots, SDS-PAGE gels or ELISPOT. The presence of a cell-mediated immunological response can be determined by proliferation assays (CD4+ T cells) or CTL (cytotoxic T lymphocyte) assays which are known in the art.

Effective doses of the nucleic acid nanoparticles or pharmaceutical compositions of the present disclosure, for the induction of an immune response, vary depending upon many different factors, including means of administration, target site, physiological state of the subject, whether the subject is a human or an animal, other medications administered, and whether treatment is prophylactic or therapeutic. Treatment dosages need to be titrated to optimize safety and efficacy, and could involve oral treatment.

In some embodiments, the nucleic acid nanoparticle or the pharmaceutical composition is administered to elicit an immune response against, for example, a human antigen, a viral antigen, a bacterial antigen, a fungal antigen, a parasitic antigen, an allergen, or a combination of such agents.

In some embodiments, the immune response is effective to treat a disease, disorder, or infection in the subject.

In some embodiments, the immune response is effective to prevent a disease, disorder, or infection in the subject.

As described herein, the disease, disorder, or infection may be selected from the group consisting of a cancer, an autoimmune disease, a bacterial infection, a viral infection, a fungal infection, an inherited genetic disorder, a metabolic disorder, a degenerative disorder, an injury causing permanent tissue damage, or a combination of such things.

In some embodiments, the disease, disorder, or infection is a cancer. The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung, bronchogenic carcinoma, Lewis lung carcinoma, lung neuroendocrine tumors, typical carcinoid, atypical carcinoid, and large cell neuroendocrine carcinoma), cancer of the peritoneum, hepatocellular cancer, gastric or stomach cancer (e.g., including gastrointestinal cancer, gastrointestinal stromal tumor pancreatic cancer, pancreatic adenocarcinoma, intraductal papillary mucinous neoplasm (IPMN), Islet cell tumors), cervical cancer (including, but not limited to, cervical adenocarcinoma), ovarian cancer (including, but not limited to, cystadenocarcinoma, ovarian embryonal carcinoma, ovarian adenocarcinoma, ovarian clear cell carcinoma, ovarian serous cystadenoma), liver cancer, bladder cancer, cancer of the urinary tract, hepatoma, breast cancer (including, but not limited to, adenocarcinoma of the breast, papillary carcinoma of the breast, mammary cancer, medullary carcinoma of the breast), colon cancer (including, but not limited to colon adenocarcinoma), colorectal cancer (including, but not limited to, rectal cancer, colorectal adenocarcinoma), endometrial cancer (including, but not limited to, uterine cancer, uterine sarcoma), salivary gland carcinoma, kidney or renal cancer (including, but not limited to, nephroblastoma or Wilms' tumor, renal cell carcinoma), prostate cancer (including, but not limited to, prostate adenocarcinoma), vulval cancer, thyroid cancer, hepatic carcinoma, anal carcinoma, penile carcinoma, skin cancer (including, but not limited to, primary or metastatic melanoma, squamous cell carcinoma, keratoacanthoma, basal cell carcinoma), multiple myeloma (including, but not limited to, smoldering multiple myeloma) and acute lymphocytic leukemia (ALL) (including, but not limited to, B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL), lymphoma such as Hodgkin lymphoma (HL) (including, but not limited to, B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, brain (e.g., high grade glioma, diffuse pontine glioma, ependymoma, neuroblastoma, meningioma, astrocytoma, oligodendroglioma; medulloblastoma, or glioblastoma), as well as head and neck cancer (including, but not limited to, head and neck squamous cell carcinoma), biliary cancer (including, but not limited to, cholangiocarcinoma), bronchus cancer, chordoma, choriocarcinoma, epithelial carcinoma, endothelial sarcoma (including, but not limited to, Kaposi's sarcoma, multiple idiopathic hemorrhagic sarcoma), esophageal cancer (including, but not limited to, adenocarcinoma of the esophagus, Barrett's adenocarinoma), Ewing sarcoma, heavy chain disease (including, but not limited to, alpha chain disease, gamma chain disease, mu chain disease), hematopoictic cancer, immunocytic amyloidosis, monoclonal gammopathy of undetermined significance, myelodysplastic syndromes, myeloproliferative disorder, agnogenic myeloid metaplasia (AMM) or myelofibrosis (MF), chronic idiopathic myelofibrosis, myeloproliferative neoplasms, polycythemia vera, rectum adenocarcinoma, essential thrombocytosis, chronic neutrophilic leukemia, hypercosinophilic syndrome, soft tissue sarcoma (e.g. malignant fibrous histiocytoma (MFH), liposarcoma, malignant peripheral nerve sheath tumor (MPNST), chondrosarcoma, fibrosarcoma, myxosarcoma), and associated metastases. Additional examples of cancer can be found in The Merck Manual of Diagnosis and Therapy, 19th Edition, § on Hematology and Oncology, published by Merck Sharp & Dohme Corp., 2011 (ISBN 978-O-911910-19-3); The Merck Manual of Diagnosis and Therapy, 20th Edition, § on Hematology and Oncology, published by Merck Sharp & Dohme Corp., 2018 (ISBN 978-O-911-91042-1) (2018 digital online edition at internet website of Merck Manuals); and SEER Program Coding and Staging Manual 2016, each of which is hereby incorporated by reference in its entirety.

In some embodiments, the disease, disorder, or infection is an infectious disease. Exemplary infectious diseases include, but are not limited to, infections of viral etiology such as human immunodeficiency virus (HIV), coronavirus, influenza, Herpes, viral hepatitis, Epstein Bar, polio, viral encephalitis, measles, chicken pox, Papilloma virus, cytomegalovirus, Rabies, Varicella, Yellow fever, West Nile virus, Ebola; infections of bacterial etiology such as pneumonia, tuberculosis, syphilis, Lyme disease, babesiosis; or infections of parasitic etiology such as malaria, trypanosomiasis, leishmaniasis, trichomoniasis, amocbiasis.

In some embodiments, the disease, disorder, or infection is an inherited genetic disease, metabolic disease, degenerative disease, and/or autoimmune disease. Such diseases are well known to those skilled in the art, and non-limiting examples include but are not limited to multiple sclerosis, type I and type II diabetes, Parkinson's disease, Alzheimer's disease, amyotrophic lateral sclerosis, heart disease, chronic obstructive pulmonary disease, osteoarthritis, degenerative disc disease, hemoglobinopathies (including, but not limited to, b-Thalassemia major, a-Thalassemia major, sickle cell anemia), mucopolysaccharidoses, mucolipidoses, osteopetrosis, Diamond-Blackfan syndrome, and various inborn errors of metabolism.

Another aspect of the present disclosure relates to a method of expressing a heterologous RNA molecule and/or protein of interest in a population of cells. This method involves contacting a population of cells with a nucleic acid nanoparticle according to the present disclosure.

Suitable methods of contacting a population of cells are well known in the art. In some embodiments, said contacting is carried out by transfection. Cells may be transfected by, e.g., electroporation, direct micro injection, or using a transfection reagent such as a cationic lipids.

Suitable DNA nanoparticles according to the present disclosure are described in detail supra.

In some embodiments, the method further involves culturing the population of cells under conditions effective to induce transcription of the encoded one or more RNA molecules and/or proteins of interest to produce an RNA molecule. In some embodiments, the RNA molecule is a mRNA molecule. Additional suitable RNA molecules are described in detail supra.

In some embodiments, the method further involves culturing the populations of cells under conditions effective to induce translation of the mRNA molecule to produce a protein of interest. Suitable proteins of interest are described in detail supra.

The contacting step may be carried out in vitro, ex vivo, or in vivo.

The nanoparticle may encode a viral antigen, a bacterial antigen, a fungal antigen, or a parasitic antigen.

In some embodiments, the population of cells is a population of mammalian cells, e.g., human cells, rodent cells, feline cells, canine cells, bovine cells, porcine cells, equine cells, etc.

The above disclosure generally describes the present invention. A more specific description is provided below in the following examples. The Examples are described solely for the purpose of illustration and are not intended to limit the scope of the present invention. Changes in form and substitution of equivalents are contemplated as circumstances suggest or render expedient. Although specific terms have been employed herein, such terms are intended in a descriptive sense and not for purposes of limitation.

### EXAMPLES

### Materials and Methods for Examples 1-4

Gene-bearing DNA origami scaffolds were prepared as previously described (Oh and Henderson, “A Comparison of Methods for the Production of Kilobase-Length Single-Stranded DNA,” DNA 2:56-67 (2022), which is hereby incorporated by reference in its entirety).

TABLE 3
Primer Sequences for PCR, aPCR, and RT-PCR

| Primer | Sequences | SEQ ID NO: |
|---|---|---|
| T7EGFP | 5′ CCA GAT ATA CGC GTT GAC ATT GAT TAT TGA 3′ | 30 |
| sense | | |
| T7EGFP | 5′ TAT ATA CAA TTT CAC ACA GGA AAC AGC TAT GA 3′ | 31 |
| anti | | |
| 3′ T7EGFP | 5′ CCA GAT ATA CGC GTT GAC ATT GAT TAT TGA /3′phos/3′ | 32 |
| blocker | | |
| RT sense | 5′ TGG TGA GCA AGG GCG AGG 3′ | 33 |
| RT anti | 5′ TCA CTT GTA CAG CTC GTC CAT GCC 3′ | 34 |

Modifications: /3′phos/: 3′ phosphorylation

### Standard PCR

Primers for amplification of the Green Fluorescent Protein (GFP) gene were designed using SnapGene and purchased from Integrated DNA Technologies (IDT, Coralville, IA). The Sequence of each primer is listed in Table 3. Phusion® DNA polymerase for the PCR reaction was purchased from New England Biolabs (NEB, Ipswich, MA).

For the generation of a duplex gene containing all components for transcription, each PCR reaction mixture was prepared in 50 μL final volume, composed of 1×Phusion® HF buffer (NEB), 200 nM dNTP mix (NEB), 500 nM sense primer (T7EGFP sense=undesired sense strand), 500 nM antisense primer (T7EGFP anti=desired antisense strand), 10 ng plasmid template (pCMV-T7-EGFP; Addgene, Watertown, MA, USA), 0.5 μL Phusion® DNA polymerase, and nuclease-free water to volume. Each PCR was performed using the following thermocycler steps: 30 s at 98° C., 30 seconds at 58° C., and 1 minute at 72° C. for 30 cycles (Oh and Henderson, “A Comparison of Methods for the Production of Kilobase-Length Single-Stranded DNA,” DNA 2(1):56-67 (2022), which is hereby incorporated by reference in its entirety).

For the generation of duplex gene missing promoters, each PCR reaction mixture was prepared in 50 μL final volume, composed of 1×Phusion® HF buffer (NEB), 200 nM dNTP mix (NEB), 500 nM sense primer (RT-sense), 500 nM antisense primer (T7EGFP anti), 10 ng pCMV-T7-EGFP (Addgene), 0.5 μL Phusion® DNA polymerase, and nuclease-free water to volume. Each PCR was performed using the following thermocycler steps: 30 seconds at 98° C., 30 seconds at 59° C., and 1 minute at 72° C. for 30 cycles (Oh and Henderson, “A Comparison of Methods for the Production of Kilobase-Length Single-Stranded DNA,” DNA 2(1):56-67 (2022), which is hereby incorporated by reference in its entirety).

The reaction products were mixed with 6×loading dye (15% Ficoll®-400, 60 mM EDTA, 19.8 mM Tris-HCl, 0.48% SDS, 0.12% Dye 1, 0.006% Dye 2, pH 8 at 25° C.; NEB) and then loaded onto a 1% agarose gel pre-stained with SYBR safe DNA dye (Invitrogen, Waltham, MA, USA). Electrophoresis was carried out at 8V/cm for 1 hour. The SYBR Safe-containing DNA was visualized using a 490 nm wavelength (bluc) transilluminator and an amber filter (Oh and Henderson, “A Comparison of Methods for the Production of Kilobase-Length Single-Stranded DNA,” DNA 2(1):56-67 (2022), which is hereby incorporated by reference in its entirety).

Asymmetric PCR (aPCR)

Primers used in aPCR were identical to those used in standard PCR. Along with sense and antisense primers, a 3′ terminal modified primer (3′ phosphorylated primers; 3′ T7EGFP blocker) was used. The 3′ T7EGFP blocker was designed using SnapGene and purchased from IDT. Its sequence and modification scheme are listed in Table 3.

For the generation of a scaffold containing all components for transcription, each aPCR reaction was carried out in 50 μL total volume, composed of 1×LongAmp® Taq buffer (60 mM Tris-SO4, 20 mM (NH4)₂SO4, 2 mM MgSO4, 3% glycerol, 0.06% IGEPAL® CA-630, 0.05% Tween® 20, pH 9.1 at 25° C.) from NEB, 500 nM T7EGFP anti, 25 nM T7EGFP sense, 475 nM 3′ T7EGFP blocker, 300 nM dNTP mix from NEB, 10 ng double-stranded GFP gene (dsT7EGFP; generated by standard PCR), 2 μL LongAmp® Taq DNA polymerase (NEB), and nuclease-frec water to final volume. Each aPCR was performed using the following thermocycler steps: 30 seconds at 94° C., 30 seconds at 58° C., and 2 minutes at 65° C. for 25 cycles (Oh and Henderson, “A Comparison of Methods for the Production of Kilobase-Length Single-Stranded DNA,” DNA 2(1):56-67 (2022), which is hereby incorporated by reference in its entirety).

For the generation of a scaffold containing all components for transcription except the promoter element, each PCR reaction was carried out in 50 μL total volume, composed of 1×LongAmp® Taq buffer (60 mM Tris-SO4, 20 mM (NH4)₂SO4, 2 mM MgSO4, 3% glycerol, 0.06% IGEPAL® CA-630, 0.05% Tween® 20, pH 9.1 at 25° C.) from NEB, 1 μM T7EGFP anti, 20 nM RT sense, 300 nM dNTP mix from NEB, 10 ng double-stranded GFP gene devoid of promoters (dsT7EGFP-T7; generated by standard PCR), 2 μL LongAmp® Taq DNA polymerase (NEB), and nuclease-free water to final volume. Each aPCR was performed using the following thermocycler steps: 30 seconds at 94° C., 30 seconds at 59° C., and 2 minutes at 65° C. for 25 cycles (Oh and Henderson, “A Comparison of Methods for the Production of Kilobase-Length Single-Stranded DNA,” DNA 2(1):56-67 (2022), which is hereby incorporated by reference in its entirety).

The reaction product was loaded onto a 1% agarose gel pre-stained with 1×SYBR Safe (Invitrogen), electrophoresed, and visualized as above.

### DNA Purification

Double-stranded DNA (dsDNA) purification. A Zymoclean Gel DNA Recovery Kit (Zymo Research, Irvine, CA) was used to extract dsDNA from agarose gels. Gel bands containing target dsDNA were removed using a clean razor blade. Three times the gel slice volume of the provided agarose dissolving/binding buffer was added to each sliced gel fragment and incubated at 55° C. on a heating block for 15 minutes. Each dissolved gel solution was transferred to the provided silica-based spin columns and centrifuged at 10,000 relative centrifugal force (rcf) for 60 seconds in a table-top centrifuge. 200 μL of ethanol-based DNA wash buffer was added to each spin column and centrifuged at 10,000 rcf for 30 seconds. A washing step was repeated before centrifuging at 10,000 rcf for 60 seconds for the complete removal of ethanol. Flow-through from all steps was discarded. After transferring each spin column to a clean microcentrifuge tube, 6-20 μL of the provided elution buffer (10 mM Tris-HCl, 0.1 mM EDTA, pH 8.5) was added directly to the matrix of each spin column followed by centrifugation at 10,000 ref for 60 seconds for DNA collection. A fraction of each purified dsDNA was mixed with 6×loading dye (NEB) and loaded onto 1% agarose gel pre-stained with 1×SYBR safe. The gel was run at 8V/cm for 1 hour. The yield of the purified dsDNA samples was evaluated by measuring band intensities relative to a known control using GelAnalyzer 19.1 available at www.gelanalyzer.com (Oh and Henderson, “A Comparison of Methods for the Production of Kilobase-Length Single-Stranded DNA,” DNA 2(1):56-67 (2022), which is hereby incorporated by reference in its entirety).

Single-stranded (ssDNA) purification. A Zymoclean Gel RNA Recovery Kit from Zymo Research was used to purify ssDNA from agarose gels. The gel bands containing target ssDNA were excised with a clean razor blade. Three times the gel slice volume of the provided agarose dissolving/binding buffer were added to each excised gel band and melted at 55° C. on a heating block for 15 minutes. Each dissolved gel solution was transferred to a provided silica-based spin columns and centrifuged at 12,000 ref for 2 minutes. 400 μL RNA Prep buffer was added to each spin column followed by centrifugation at 12,000 ref for 1 minute. Washing was carried out by the addition of 800 μL ethanol-based wash buffer followed by centrifugation at 12,000 rcf for 30 seconds. After repeating the washing step with 400 μL ethanol-based wash buffer, each spin column was centrifuged at 12,000 rcf for 2 minutes to remove residual ethanol. Flow-through in all steps was discarded. After transferring each spin column to clean microcentrifuge tubes, 6-20 μL of provided nuclease-free water was added directly to the column matrix, and the spin columns were centrifuged at 10,000 ref for 1 minute for retentate collection. A fraction of each purified ssDNA was mixed with 6×loading dye (NEB) and the yield was estimated by gel electrophoresis as described above (Oh and Henderson, “A Comparison of Methods for the Production of Kilobase-Length Single-Stranded DNA,” DNA 2(1):56-67 (2022), which is hereby incorporated by reference in its entirety).

### DNA Nanoparticle Construction

DNA nanoparticles were designed using caDNAno (www.cadnano.org) and staples were purchased from IDT (Tables 4 and 5). DNA nanoparticles were prepared by mixing single-stranded GFP gene (ssT7EGFP or ssT7EGFP-T7; generated by aPCR) to a final concentration of 91.4 nM and each staple to a final concentration of 457 nM in 1×TAE buffer supplemented with 12.5 mM Mg(OAc)₂ (TAEM) in a final volume of 50 μL. The staple set for each DNA nanoparticle is listed in Table 6. The mixture was incubated at 90° C. for 10 minutes in a water bath followed by gradual cooling to room temperature. Products of this reaction were mixed with 6×loading dye (NEB) and then loaded onto 1% agarose gel containing 12.5 mM Mg(OAc)₂ pre-stained with 1×SYBR Safe DNA dye (Invitrogen). Electrophoresis was carried out in TAEM buffer at 6V/cm for 90 minutes. The gel was visualized as above. DNA origami was purified using a Freeze 'N Squeeze™ DNA Gel Extraction Spin Column (Bio-Rad, Hercules, CA, USA). Gel bands containing target DNA origami were sliced and removed using a clean razor blade, then transferred to Freeze 'N Squeeze™ DNA Gel Extraction Spin columns. Spin columns containing target DNA origami gel slices were incubated at −20° C. for 5 minutes followed by centrifugation at 13,000 rcf in a table-top centrifuge for 3 minutes at room temperature. The concentration of the purified DNA origami samples was measured using a NanoDrop™ instrument.

TABLE 4
T7GHL Staple Sequences

| Staple Names | Sequences | SEQ ID NO |
|---|---|---|
| T7GHL 1 | AAG CTG GAT CCG CCC CTG AGC ACC CAG TGT TCG TG | 35 |
| T7GHL 2 | AGT TCC GCG TTA CAT AAC TTA CGG TAA ATG GCC CGC | 36 |
| | CTG GCT | |
| T7GHL 3 | GCT GCC CCC ACC CTC GTG ACC GGG CGA C | 37 |
| T7GHL 4 | AGG TGC CGC CAT CTT GCT GGA CCG CCC TAG GTG AA | 38 |
| T7GHL 5 | ATG GGT GGA GTA TTT ACG GTA AAC TGC CCA CTT GGC | 39 |
| | AGT ACA | |
| T7GHL 6 | CGG TAG GCG TGT ACG GTG GGA GGT C | 40 |
| T7GHL 7 | CGC CGA CTC GAC TTA GTT CGA ACC CTG ATG CAC CA | 41 |
| T7GHL 8 | ATC GCT ATT ACC ATG GTG ATG CGG TTT TGG CAG TAC | 42 |
| T7GHL 9 | CGA GGA CAC ATC CTA CAA GAC TGC TTC AAA GCT GA | 43 |
| T7GHL 10 | AGG ACA GCT GTT TCC TGT GTG GCA AGG GCG AGG AGG | 44 |
| | TTC ATC CCT ACG G | |
| T7GHL 11 | CCA GAT ATA CGC GTT GAC ATT GAT TAT TGA CTA GTT | 45 |
| | ATT AAT | |
| T7GHL 12 | CCC CGA CGG ACG ACG GCA ACT GGG GCA C | 46 |
| T7GHL 13 | TGT TCC CAT AGT AAC GCC AAT AGG GAC TTT CCA TTG | 47 |
| | ACG TCA | |
| T7GHL 14 | AAT CAT GCC GGG GTG GTG CCC CTA CGG CGC CGC TA | 48 |
| T7GHL 15 | CTT CAA GAG TAC AAT CTT CAA CAC ATG AGA GGG CG | 49 |
| T7GHL 16 | GAA GGC TTA TCA TGG C | 50 |
| T7GHL 17 | TAT ATA AGC AGA GCT GGT TTA GTG AAC CGT CAG ATC | 51 |
| | CGC TAG | |
| T7GHL 18 | ATG CCA CAT CCT GGC TAG AGC CAA TAG CCT GTC CT | 52 |
| T7GHL 19 | CCT CTA GTC GAG CTG GAC GGC CGA GGG CAG CAG CA | 53 |
| T7GHL 20 | TTC CTA AGA CTG TGC CGG GAT ACC ACT AAC AAC AT | 54 |
| T7GHL 21 | TGT CCG GGA CGT AAC TCG ATA TGC GGT GCC CTG GA | 55 |
| T7GHL 22 | AGT AAT CAA TTA CGG GGT CAT TAG TTC ATA GCC CAT | 56 |
| | ATA TGG | |
| T7GHL 23 | AGC CAC AGG CAT CAG AGC AAA GAC CCC ACG ATC ACA | 57 |
| | TGG TCC GTT GTT T | |
| T7GHL 24 | GAC CGC CCA ACG ACC CCC GCC CAA TGA CGT CAA TAA | 58 |
| | TGA CGT A | |
| T7GHL 25 | AGA ACC AGC TGG GGA CGG CCA CA | 59 |
| T7GHL 26 | AGA TCC GCG GCC GCT AAT ACG ACT CAC TAT AGG GAG | 60 |
| | AGC CGC CAC C | |
| T7GHL 27 | TCA AGT GTA TCA TAT GCC AAG TAC GCC CCC TAT TGA | 61 |
| | CGT CAA T | |
| T7GHL 28 | GAC GGC AGG CAG CGG CTG CTG CCC GAC ACA CTC TC | 62 |
| T7GHL 29 | AGT CTC CAC CCC ATT GAC GTC AAT GGG AGT TTG TTT | 63 |
| | TGG CAC | |
| T7GHL 30 | CGT GCA GCC GCG CCG AGG TGA CAA GGA G | 64 |
| T7GHL 31 | ACA AGC AGA AGA ACA CGT CTA ACG TCC ATT CAA GTT | 65 |
| | TCA GCG | |
| T7GHL 32 | GAC GGT AAA TGG CCC GCC TGG CAT TAT GCC CAG TAC | 66 |
| | ATG ACC | |
| T7GHL 33 | ATC AAT GGG CGT GGA TAG CGG TTT GAC TCA CGG GGA | 67 |
| | TTT CCA | |
| T7GHL 34 | TTA TGG GAC TTT CCT ACT TGG CAG TAC ATC TAC GTA | 68 |
| | TTA GTC | |
| T7GHL 35 | CCA TTG ACG CAA ATG GG | 69 |
| T7GHL 36 | CCC TGA ACT GTT CAG TCA TAG CAA GGG GAG GAA AT | 70 |
| T7GHL 37 | TGC ATC GTT AAA CCA CGA GCT GCC CCG TTG CAG CT | 71 |
| T7GHL 38 | CGA CTT CGG AGC GCA CCA TCT CTA CAA C | 72 |
| T7GHL 39 | GGC ATG GCG CTG ATC AGC CTC TAA AAT GGA GGA TTG | 73 |
| | GGA AGA TTG GCG T | |
| T7GHL 40 | ACC CTG GCG AGC TGA AGG GCA CAC TAC CCC CCA TCG | 74 |
| | GCG ACG GTA CAA G | |
| T7GHL 41 | GCC CCT CTC CTT GAG GCT CTA TGG CTT CTG AGG | 75 |
| T7GHL 42 | CAA AAT CAA CGG GAC TTT CCA AAA TGT CGT AAC AAC | 76 |
| | TCC GCC | |
| T7GHL 43 | TGA CCG GCA TCA CCA TTG AGT CAT TGT CGT GGG GC | 77 |
| T7GHL 44 | ACC GCC GCC TTC TAG TTG CCA ACT CCC AAG GCA TGC | 78 |
| | TGG GGA CCG TCG A | |
| T7GHL 45 | CCG GCA AAT GGT GAA AAT TGT GGG TGG GTG AGT AGG | 79 |
| | TGT CAT | |

TABLE 5
T7GHL BP Staple Sequences

| Staple Name | Sequence | SEQ ID NO |
|---|---|---|
| T7GHL BP 1 | GCA GCA CTG TCC GGG ACG TAA CTC GAT ATG CGG TG | 80 |
| T7GHL BP 2 | AAG CTG AGT GCA GTG GAC GAC | 81 |
| T7GHL BP 3 | CTG TGT GGC AAG GGA TAG GGA AGG GCA TGG CAG CG | 82 |
| T7GHL BP 4 | CCA AGT CTC CAC CCC ATT GAC GTC AAT GGG AGT TTG | 83 |
| | TTT TGG C | |
| T7GHL BP 5 | TGC ACC AGT GAC CAC CGC GCC | 84 |
| T7GHL BP 6 | GTA ACG CCA ATA GGG ACT TTC CAT TGA CGT CAA TGG | 85 |
| | GTG GAG TAT T | |
| T7GHL BP 7 | GAG GAT TTC CTA ATA CTG TGC CGG GAT CCC ACT AC | 86 |
| T7GHL BP 8 | ACA TCT ACG TAT TAG TCA TCG CTA TTA CCA TGG TGA | 87 |
| | TGC GGT TTT G | |
| T7GHL BP 9 | ACC AAA ATC AAC GGG ACT TTC CAA AAT GTC GTA ACA | 88 |
| | ACT CCG CCC CA | |
| T7GHL BP 10 | GGC AAC TAA GGA GGT AAT ACG AGC CTC GAA AAT GA | 89 |
| T7GHL BP 11 | CAC CCT CCC GGC AAA TGG TGA AAA TTG TGG GTG GG | 90 |
| T7GHL BP 12 | AAG TAC GCC CCC TAT TGA CGT CAA TGA CGG TAA ATG | 91 |
| | GCC CGC CTG G | |
| T7GHL BP 13 | GTG GGG CGC ATC GCT AAA CCC CGA GCT GCC CCG TG | 92 |
| T7GHL BP 14 | GCA TGG AGC TGA TCA CTC ACT CGA GGA GGT TCA TC | 93 |
| T7GHL BP 15 | GGA AAT TAG GAC AGC TGT TTC | 94 |
| T7GHL BP 16 | TTG ACG CAA ATG GGC GGT AGG CGT GTA CGG TGG | 95 |
| | GAG GTC TAT ATA A | |
| T7GHL BP 17 | GAG GGC GCC CGA CCG GAG CGC | 96 |
| T7GHL BP 18 | AGT AAT CAA TTA CGG GGT CAT TAG TTC ATA GCC CAT | 97 |
| | ATA TGG A | |
| T7GHL BP 19 | CCT GGA ACT GGG GAC CGT CGA | 98 |
| T7GHL BP 20 | CCA GAT ATA CGC GTT GAC ATT GAT TAT TGA CTA GTT | 99 |
| | ATT AAT | |
| T7GHL BP 21 | TGG TCC TTT GTT TGG TGA ACC GGA CGG CCG AGG GC | 100 |
| T7GHL BP 22 | CCG CTA CAT GCC ACA TCC TGG CTA GAG CCA ATA GC | 101 |
| T7GHL BP 23 | GAA GGC TTA CAA CAT GGT TTA CCC CTC CCC TTG AC | 102 |
| T7GHL BP 24 | CCT GGT GAA CAC TAC CAG | 103 |
| T7GHL BP 25 | CCG CCG CCT TCT AGA GAT CCG GGT GCC CCT ACG GC | 104 |
| T7GHL BP 26 | GTT CCG CGT TAC ATA ACT TAC GGT AAA TGG CCC GCC | 105 |
| | TGG CTG ACC GC | |
| T7GHL BP 27 | CTG AGC ACT TCA AGC ATC CTG TCT TCA AGC TTC AG | 106 |
| T7GHL BP 28 | ACA ACA TCC GAC AAA CTC TCG | 107 |
| T7GHL BP 29 | CCT CTA GTC GAG CTG TCA GAT AGC TGG AGG CAT CA | 108 |
| T7GHL BP 30 | AAT CAT GCC GGG GTC GGC CGC ACG GCA AAT CCG CC | 109 |
| T7GHL BP 31 | CTA CGG CCC CTG AAC TGT TCA GTC ATA GCA AGG GG | 110 |
| T7GHL BP 32 | TTC AGC GGA CTT CTC ATG CCC | 111 |
| T7GHL BP 33 | GGC TCT ATG GCT TCA GAA CCA | 112 |
| T7GHL BP 34 | GCA GTA CAT CAA TGG GCG TGG ATA GCG GTT TGA CTC | 113 |
| | ACG GGG ATT T | |
| T7GHL BP 35 | GAA CAC CCC CAT CGC GCC GAC CCG CAT CAG TTC GAC | 114 |
| | CTG GCC | |
| T7GHL BP 36 | CCA ACG ACC CCC GCC CAA TGA CGT CAA TAA TGA CGT | 115 |
| | ATG TTC CCA TA | |
| T7GHL BP 37 | TGT CCT TGG GAA GAT TGG CGT | 116 |
| T7GHL BP 38 | GAC CGG TCA TCA TCA CCA TCA CCA | 117 |
| T7GHL BP 39 | CTG CTG CCG AGG ACC GAC TTC ACA AGA CCC CTG AC | 118 |
| T7GHL BP 40 | CAT TAT GCC CAG TAC ATG ACC TTA TGG GAC TTT CCT | 119 |
| | ACT TGG CAG T | |
| T7GHL BP 41 | AGC AAA GGA AGA ACG TAC AAC ACG TCC AAC ATG AA | 120 |
| T7GHL BP 42 | ACC ATC TGG GCA CAC CGC TAG TTG CCA GCT CCC AC | 121 |
| T7GHL BP 43 | GAG GTG AGA GCT GAG AGC CGC TTG AGT TAT TGT CTG | 122 |
| | AGT AGG TTC TGG G | |
| T7GHL BP 44 | TAC GGT AAA CTG CCC ACT TGG CAG TAC ATC AAG TGT | 123 |
| | ATC ATA TGC C | |
| T7GHL BP 45 | GCT GGG GAC GGC CAG CAG AGC GCC ACA AAT GGC CG | 124 |
| T7GHL BP 46 | AGG CAT GGG TGC CAC CAT CTG GCT GGA GCG CCC TG | 125 |
| T7GHL BP 47 | TGC AGC TGC GAC GGT ACA AGT | 126 |
| T7GHL BP 48 | ACA AGC AAC CCC AAG ATC ACA | 127 |
| T7GHL BP 49 | AGG TGA ACC CAG TCT TCG TGA | 128 |

TABLE 6
Staple List for DNA Nanoparticle Construction

| DNA | |
|---|---|
| Nanoparticle | Staple List |
| T7GHL PO | T7GHL 2, 5, 6, 8, 11, 13, 17, 22, 24, 26, 27, |
| | 29, 32-35, 42 |
| T7GHL HS | T7GHL 1-3, 5, 6, 8, 10-14, 17, 19, 22-35, 38-44 |
| T7GHL FS | T7GHL 1-45 |
| T7GHL BP | T7GHL BP 1-49 |
| T7GHL − T7 | T7GHL 1-45 |

In vitro Transcription (IVT)

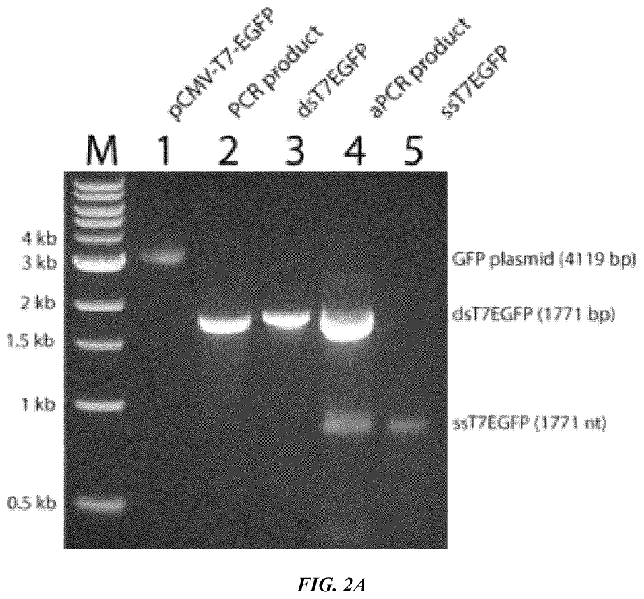

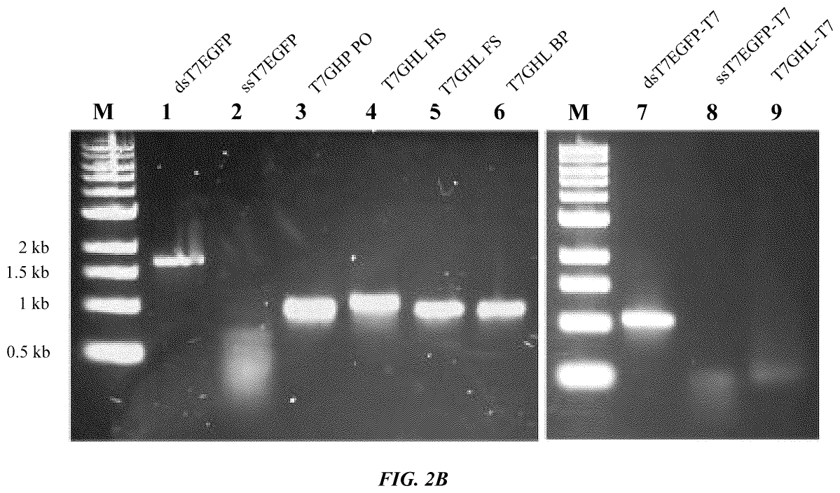

All IVT reactions were carried out in 20 μL total volume using a HiScribe® T7 Quick High Yield RNA Synthesis Kit (NEB). Each reaction contained 10 μL NTP buffer mix (10 mM each NTP; NEB), 10 ng DNA template (linearized GFP plasmid (LpCMV-T7-EGFP), dsT7EGFP, ssT7EGFP, each DNA nanoparticle (designated as described in Table 7 and in the brief description of FIG. 2B), 2 μL T7 RNA polymerase mix, and nuclease-free water to volume. Each reaction was carried out at 37° C. for 2 hours. For T7GHL FS, T7GHL PO, and T7GHL HS, a reaction time course of 30, 60, 90, and 120 minutes was carried out for a preliminary comparison of the relative transcription rates of these constructs and substrate longevity.

TABLE 7
Names of DNA scaffolds and DNA Nanoparticles

| Name | Contents |
|---|---|
| ssT7EGFP | Single-stranded antisense strand for nanoparticle scaffold |
| SST7EGFP − | Single-stranded antisense strand for nanoparticle scaffold lacking the T7 |
| T7 | promoter |
| T7GHL PO | DNA nanoparticle with a linear duplex promotor and single-stranded GFP gene; |
| | variant of T7-based GFP gene Honeycomb structure with a Linear promoter, |
| | the Promoter Only being in duplex form within the structure |
| T7GHL HS | DNA nanoparticle with a linear duplex promotor and a half complement of |
| | staples (i.e., loosely folded); variant of T7-based GFP gene Honeycomb |
| | structure with a Linear promoter, the structure folded with a Half-Set of staples |
| T7GHL FS | DNA nanoparticle with a linear duplex promotor and a full complement of |
| | staples (i.e., tightly folded); variant of T7-based GFP gene Honeycomb |
| | structure with a Linear promoter, the structure folded with a Full-Set of staples |
| T7GHL BP | DNA nanoparticle with a linear duplex promotor that is buried within a folded |
| | architecture with a full complement of staples (i.e., buried promoter, tightly |
| | folded); variant of T7-based GFP gene Honeycomb structure with a Linear |
| | promoter; the structure with Buried Promoter inside the structure |
| T7GHL − T7 | DNA nanoparticle with a full complement of staples but lacking the T7 |
| | promoter sequence; variant of T7-based GFP gene Honeycomb structure with |
| | a Linear promoter, the structure lacking (−) T7 promoter |

In Vitro Transcription (IVT) on DNA Nanoparticles with Increasing Magnesium (Mg²⁺) Concentration

All IVT reactions were carried out in 20 μL total volume using HiScribe® T7 Quick High Yield RNA Synthesis Kit (NEB). Each reaction component contains 10 μL NTP buffer mix (10 mM each NTP; NEB), 10 ng DNA template (dsT7EGFP and T7GHL BP), 2 μL T7 RNA polymerase mix, 1×TAEM (1×TAE buffer containing 12.5 mM Mg(OAc)₂ to give final additional Mg²⁺ concentration of 0, 1.6, 3.2, and 5 mM, and nuclease-free water to volume. For evaluation of structural integrities of DNA nanoparticles in IVT reaction conditions, each reaction was prepared with 10 μL NTP buffer mix (10 mM each NTP; NEB), 10 ng DNA template (T7GHL FS), 1×TAEM to give a final additional Mg²⁺ concentration of 0.9, 1.9, 3.8, and 5 mM, and nuclease-free water to volume. Each reaction was carried out at 37° C. for 2 hours. IVT products were purified using Monarch® RNA Cleanup Kit (NEB), electrophoresed, and visualized as described in the manuscript.

### Purification of IVT Products

Following IVT, 30 μl of nuclease-free water was added to each IVT reaction product to increase the reaction volume. Each IVT product was then purified using a Monarch® RNA Cleanup Kit (NEB). 100 μl RNA binding buffer was added to each 50 μl IVT product followed by the addition of 150 μl of absolute ethanol. Each mixture was then transferred to the provided silica-based spin columns and centrifuged in a table-top centrifuge. 500 μl of ethanol-based DNA wash buffer was added to each spin column and centrifuged. The washing step was repeated once more and the flow-through from all steps was discarded. Each spin column was transferred to a clean microcentrifuge tube and 10 μl of the provided nuclease-free water was added directly to the matrix of each spin column followed by centrifugation for DNA collection. All centrifugation steps were carried out in 16,000 rcf for 60 seconds.

### Evaluation of IVT Products

IVT products from all DNA samples were analyzed by gel electrophoresis. Purified GFP PCR and linearized GFP plasmid IVT products were diluted 100-fold to avoid over-staining. Purified RNA products were mixed with an equal volume of 2×RNA loading dye (95% formamide, 0.02% SDS, 0.02% bromophenol blue, 0.01% Xylene Cyanol, 1 mM EDTA; NEB). Samples were heated to 70° C. for 10 minutes prior to gel loading. Electrophoresis was carried out at 8V/cm for 1 hour. The gels were post-stained with 1×TAE solution containing 1×SYBR gold (Invitrogen) for 2 hours. The stained gels were visualized using a 490 nm wavelength transilluminator and an amber filter.

### Reverse Transcription PCR (RT-PCR)

RNA templates for RT-PCR were prepared by carrying out an IVT reaction using each DNA template (LpCMV-T7-EGFP, dsT7EGFP, ssT7EGFP, T7GHL PO, T7GHL HS, T7GHL FS, and T7GHL BP) as described above. To minimize the possible background signal caused by the presence of residual DNA template, 1 ng of each DNA template (rather than 10 ng) was used for these reactions, and the incubation time was extended to overnight to maximize RNA production under these conditions. Following IVT, DNA was hydrolyzed by treatment with DNase. A 50 μL DNase mixture was prepared by mixing 20 μL IVT product with 2 μL RNase-free DNase-I and nuclease-free water to volume. All DNase reactions were carried out at 37° C. for 30 minutes. DNase-treated RNA products were purified using a Monarch® RNA Cleanup Kit (NEB) as described above. Primers for amplification of the GFP gene were designed using SnapGene and purchased from IDT. The sequence of each primer is listed in Table 3. RT-PCR was performed using a OneTaq® One-step RT-PCR kit (NEB). Each RT-PCR mixture was carried out in 50 μL final volume, containing 1×Quick-Load® OneTaq One-step reaction mix (1.6 mM MgCl2, 250 nM dNTP mixture), 400 nM sense primer (RT-sense), 400 nM antisense primer (RT-anti), 1 μL of each purified RNA product, 1×OneTaq® One-step enzyme mix (ProtoScript® II reverse transcriptase, OneTaq® Hot Start DNA polymerase, Murine RNase inhibitor, and stabilizer), and nuclease-free water to volume. To assess any contribution to PCR signal from residual DNA resulting from incomplete DNase hydrolysis, negative controls were prepared in parallel. These reactions contained the same components as RT-PCR mixtures but substituted OneTaq® Hot Start DNA polymerase for the OneTaq® One-step enzyme mix (i.e., lacking reverse transcriptase altogether). In this case, any signal represented the amplification of residual DNA remaining after DNase treatment. Reactions were carried out by treating each RT-PCR mixture at 48° C. for 30 minutes followed by PCR. Each PCR was performed using the following thermocycling steps: 30 seconds at 94° C., 30 seconds at 60° C., and 1 minute at 68° C. for 15 cycles. Each product was loaded onto a 1% agarose gel pre-stained with SYBR-safe DNA dye (Invitrogen). The gel was electrophoresed and visualized as above.

RT-PCR with Increasing DNase Treatment Duration

For the preparation of substrate for RT-PCR, IVT reactions were performed prior to RT-PCR. All IVT reactions were carried out in 20 μL, containing 10 μL NTP buffer mix (10 mM each NTP; NEB), 1 ng DNA template (dsT7EGFP), 2 μL T7 RNA polymerase mix, and nuclease-free water to volume. All IVT reactions were carried out at 37° C. overnight. DNase mixtures were prepared by mixing 20 μL of each IVT product, 2 μL DNase-1, and nuclease-free water to volume. DNase treatment was carried out at 37° C. for 0, 5, 15, and 30 minutes respectively. DNase-treated IVT products were then purified using Monarch® RNA Cleanup Kit (NEB) as described in the paper. RT-PCR was performed using OneTaq® One-step RT-PCR Kit (NEB). All RT-PCR mixtures were carried out in 50 μL. Each mixture contained 1×Quick-Load® OneTaq One-step reaction mix (1.6 mM MgCl2, 250 nM dNTP mix; NEB), 400 nM sense primer (RT-sense; IDT), 400 nM antisense primer (RT-anti; IDT), 1 μL each purified RNA product, 1×OneTaq® One-step enzyme mix (ProtoScript® II reverse transcriptase, OneTaq® Hot Start DNA polymerase, Murine RNase inhibitor, and stabilizer), and nuclease-free water to volume. In parallel to RT-PCR mixtures, no RT mixtures (negative controls) were prepared in 50 μL, containing the same components as RT-PCR mixtures. On no RT mixtures, instead of 1×OneTaq® One-step enzyme mix, 0.5 μL OneTaq® Hot Start DNA polymerase (NEB) was used. Reverse transcription was carried out by treating each RT-PCR mixture at 48° C. for 30 minutes followed by PCR. Each PCR was performed using the following thermocycling steps: 30 seconds at 94° C., 30 seconds at 60° C., and 1 minute at 68° C. for 15 cycles. Each product was loaded onto 1% agarose gel pre-stained with SYBR-safe DNA dye (Invitrogen). The gel was electrophoresed and visualized as described in the manuscript.

### Transmission Electron Microscopy (TEM)

TEM was performed as previously described (Mathur and Henderson, “Programmable DNA Nanosystem for Molecular Interrogation,” Sci. Rep. 6:27413 (2016), which is hereby incorporated by reference in its entirety). Briefly, samples for TEM imaging were prepared in a concentration range of 0.5 nM to 5 nM. 12 μL of the sample was placed on glow-discharged carbon-coated 400 mesh copper TEM grids. After two minutes of incubation, the sample solution was removed using filter paper and replaced with 12 μL of freshly prepared uranyl formate negative staining solution. The stain was removed after 30 seconds, and the grids were air-dried. TEM images were acquired at 25,000× magnification using a JEOL 1230 TEM (Peabody, Ma, USA) equipped with a Gatan Inc. 2 k×2 k Ultrascan camera (Pleasanton, CA, USA) (Mathur and Henderson, “Programmable DNA Nanosystem for Molecular Interrogation,” Sci. Rep. 6:27413 (2016), which is hereby incorporated by reference in its entirety).

### Example 1-DNA Nanoparticle Construction

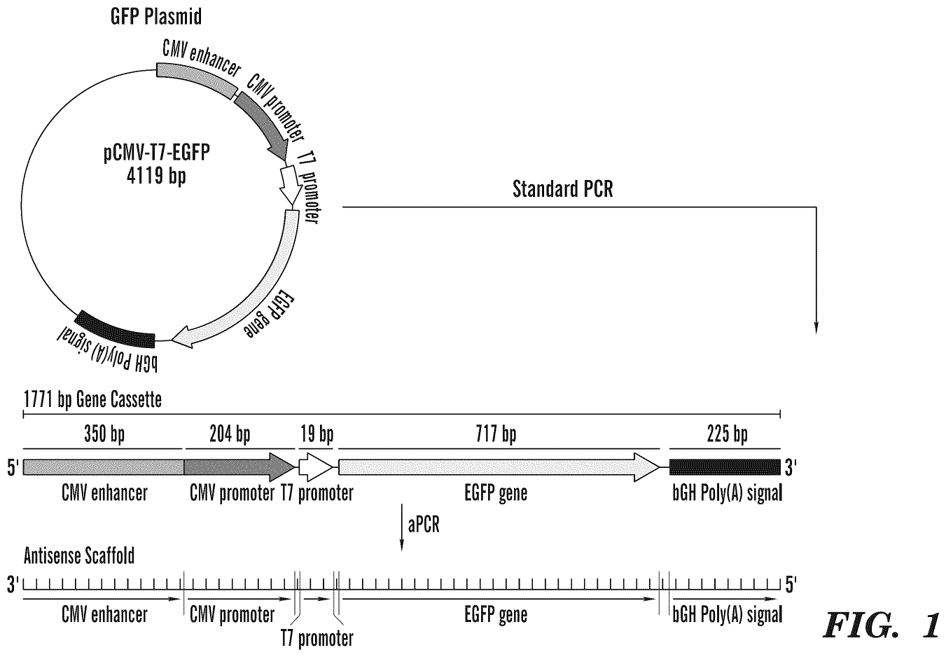

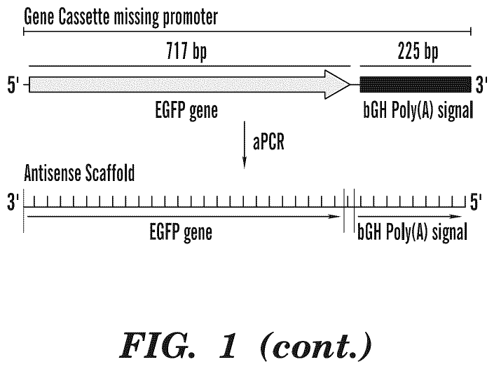

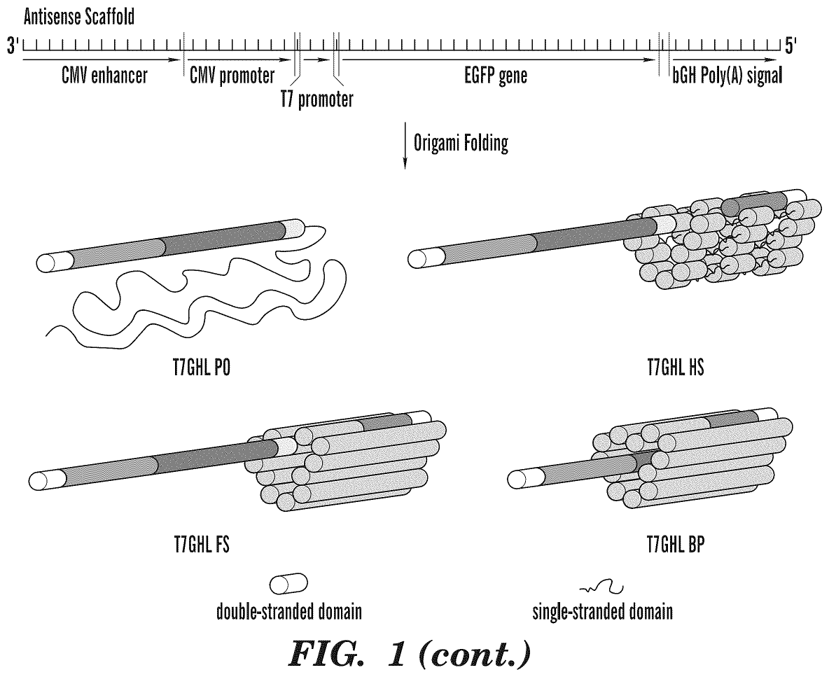

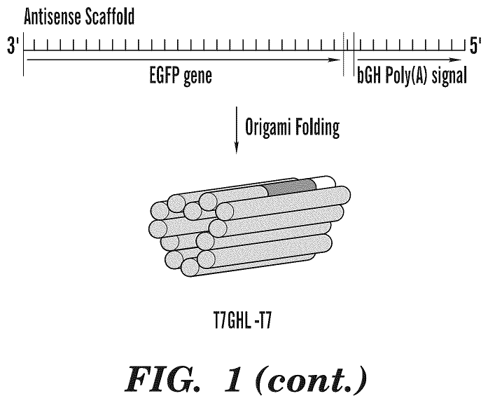

The overall process of generating the DNA nanoparticles is illustrated in FIG. 1. For the construction of potentially transcription-capable DNA nanoparticles, a custom scaffold was synthesized that contained the required gene sequence elements for expression. These scaffolds included a T7 promoter, the GFP gene, and a poly A signal (a CMV promoter and enhancer, not relevant to the present study, were also present). Conventional PCR followed by asymmetric PCR (aPCR) was performed to produce the single-stranded scaffold containing the GFP gene cassette, designated ssT7EGFP and ssT7EGFP-T7, the latter lacking the T7 promotor as a negative control (Table 3). The same primers were used for both PCR and aPCR, but in the case of the aPCR, an unequal ratio of primers was employed to enhance the production of the desired strand (the genetically defined antisense strand). A 3′ terminal blocker was used along with both sense and antisense primers to optimize the production of the desired strand by aPCR. Electrophoretic analysis of the products of this process revealed two bands estimated to be ˜ 1700 bp and ˜ 900 bp in length as determined by co-migration with dsDNA markers (FIG. 2A). Both the dsT7EGFP and the ssT7EGFP are 1771 nucleotides in length. However, the single-stranded form migrates faster under non-denaturing conditions presumably due to internal base-pairing. Thus, the strong DNA band at ˜ 1700 bp is interpreted as being dsT7EGFP and the band at approximately 900 bp to be the desired ssT7EGFP. This interpretation was corroborated by the difference in SYBR Safe DNA dye color and staining efficiency (Oh and Henderson, “A Comparison of Methods for the Production of Kilobase-Length Single-Stranded DNA,” DNA 2(1):56-67 (2022), which is hereby incorporated by reference in its entirety) and further validated by the use of the presumed ssDNA band for the successful construction of DNA origami nanoparticles.

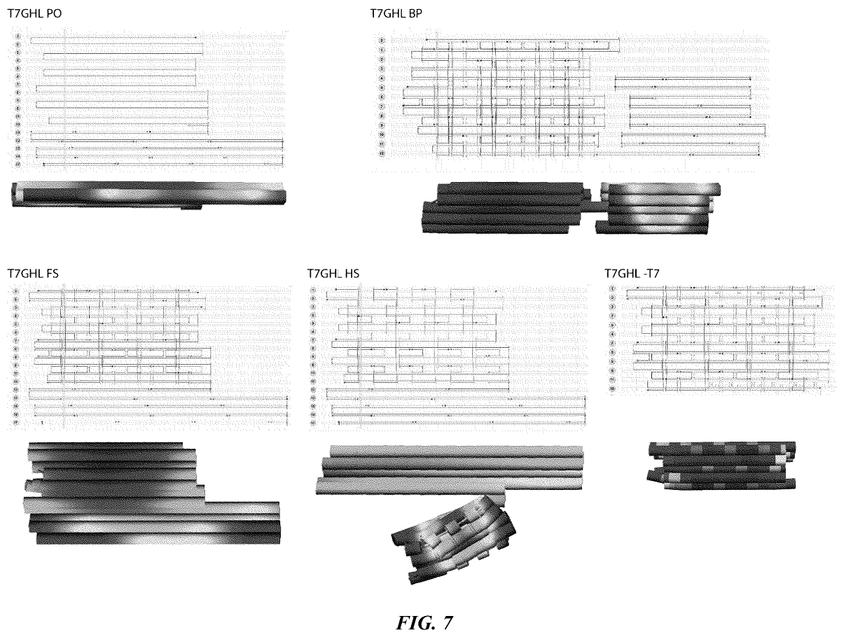

The program caDNAno (Douglas et al., “Rapid Prototyping of 3D DNA-Origami Shapes with caDNAno,” Nucleic Acids Res. 37(15):5001-5006 (2009), which is hereby incorporated by reference in its entirety) was employed to design the DNA nanoparticles used in herein. ssT7EGFP was used to construct nanoparticle architectures designated T7GHL PO, T7GHL HS, T7GHL FS, and T7GHL BP, and ssT7EGFP-T7 was used to construct T7GHL-T7 (FIG. 2B, and FIG. 7). Details regarding the acronyms of the DNA nanoparticles are listed in Table 3. A schematic diagram of the structures of each DNA nanoparticle is also provided in FIG. 1. These nanoparticles were designed to form a cylindrical shape, maintaining a crossover pattern that was relatively consistent throughout various forms of this general structure. With the exception of a T7GHL BP in which the T7 promoter is buried within the nanoparticle architecture, the promoter of the GFP gene constructs was configured as a linear (i.e., no crossovers) double-stranded DNA helix adjacent to the cylindrical core (FIG. 7). The intent of external positioning of the T7 promoter was duc to a naïve assumption that it was advantageous to optimize accessibility to the promoter by T7 polymerase. To test the validity of this assumption the T7GHL BP construct was designed with the promotor sequestered inside the cylindrical architecture with crossover density consistent with the rest of the nanoparticle architecture. Single-stranded hairpins were positioned at the ends of each double helix in all constructs to facilitate the folding process (FIG. 7).

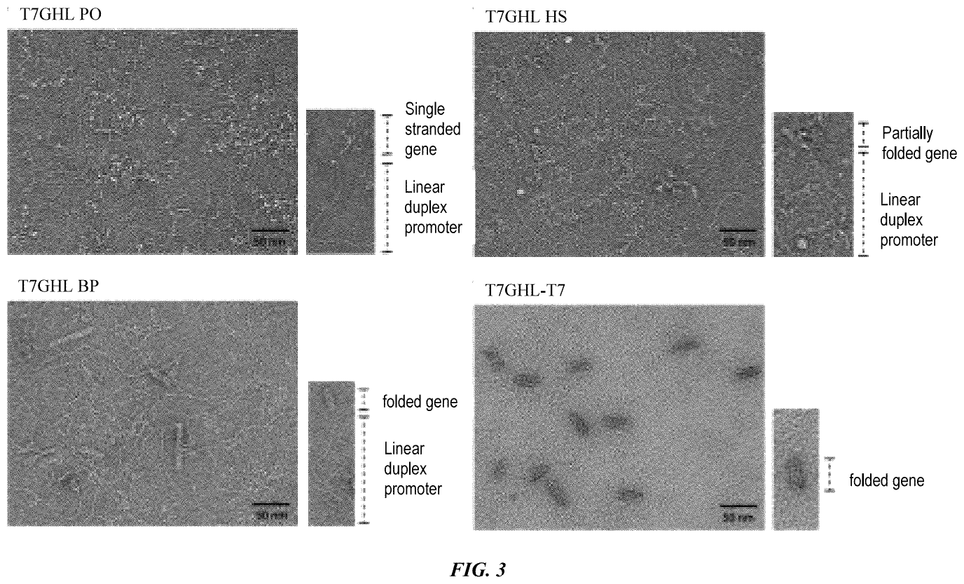

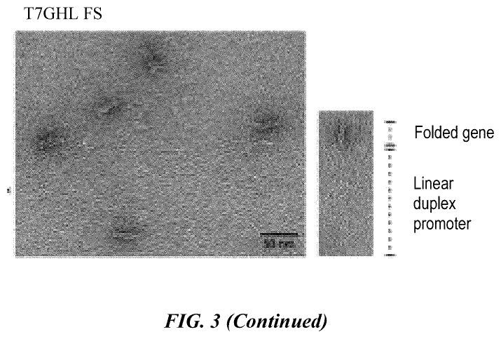

To assess the efficiency of transcription as a function of the number of crossovers employed (“compactness”), DNA nanoparticles were constructed with varying staple density (FIG. 7). Also, to evaluate whether the IVT products were the result of promoter-based transcription rather than random priming, one of the nanoparticles was constructed with a scaffold that was devoid of any T7 promoter sequence elements. Electrophoretic analysis of the DNA nanoparticle constructs revealed band shifts that were distinct from ssT7EGFP and dsT7GFP (FIG. 2B), indicating the successful self-assembly of the intended DNA nanoparticles. To minimize contamination from staples and incomplete or aberrantly folded structures that might hamper the interpretation of IVT data (Lin et al., “Purification of DNA-Origami Nanostructures by Rate-Zonal Centrifugation,” Nucleic Acids Res. 41(2):c40 (2013), which is hereby incorporated by reference in its entirety), all constructs were gel purified (FIG. 2B). Freeze-and-squeeze gel purification is a commonly used method to obtain intact ultrapure DNA nanoparticles (Lin-Shiao et al., “CRISPR-Cas9-Mediated Nuclear Transport and Genomic Integration of Nanostructured Genes in Human Primary Cells,” Nucleic Acids Res. 50(3): 1256-1268 (2022) and Shaw et al., “Purification of Functionalized DNA Origami Nanostructures,” ACS Nano. 9:4968-4975 (2015), which is hereby incorporated by reference in its entirety). Thus, the freeze-and-squeeze gel purification method was employed for the purification of DNA nanoparticles used in the study. Confirmation of the retention of the structural integrity of the DNA nanoparticles was obtained by TEM imaging of gel-purified samples (FIG. 3).

### Example 2-In Vitro Transcription (IVT)

IVT analysis was performed using the T7 promotor and a commercial T7 polymerase IVT kit (NEB). Positive controls included a linearized GFP plasmid (LpCMV-T7-EGFP) and the linear duplex GFP PCR product (dsT7EGFP) to assess the expected maximum degree of RNA production. A single-stranded antisense strand (i.e., the bare origami scaffold; ssT7EGFP) was used as a negative control based on the reported requirement for a double-stranded T7 promoter for successful transcription (Arnaud-Barbe et al., “Transcription of RNA Templates by T7 RNA Polymerase,” Nucleic Acids Res. 26(15):3550-3554 (1998) and Sarcar and Miller, “A Specific, Promoter-Independent Activity of T7 RNA Polymerase Suggests a General Model for DNA/RNA Editing in Single Subunit RNA Polymerases,” Sci. Rep. 8(1): 13885 (2018), which are hereby incorporated by reference in their entirety). The IVT reactions from positive controls (dsT7EGFP and LpCMV-T7-EGFP) generated substantial quantities of RNA products and, therefore, these products were diluted 100-fold for clear visualization by electrophoresis. Banding patterns on non-denaturing (native) and denaturing gels were virtually identical, thus, the bulk of these analyses was carried out under non-denaturing conditions. Size estimates were based on a calibrated RNA ladder from a commercial supplier (NEB).

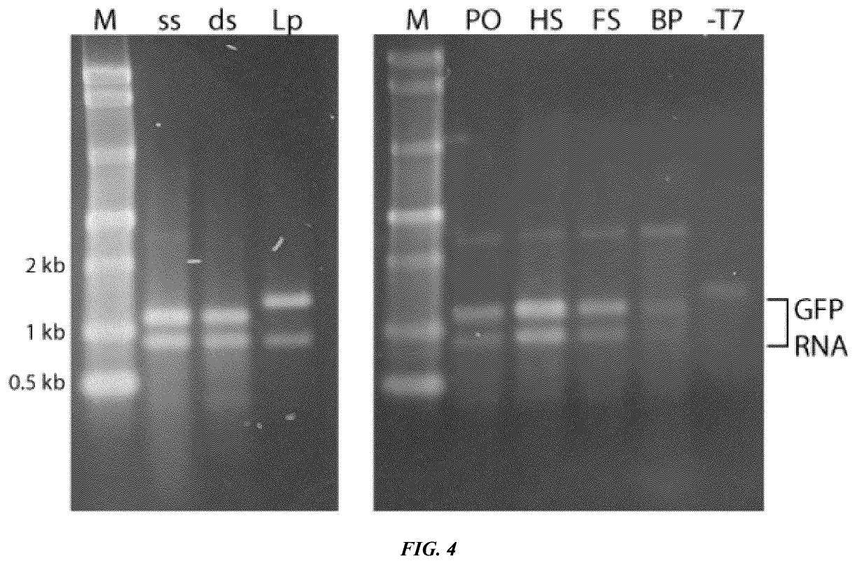

Electrophoretic analysis of IVT products revealed two RNA bands with approximate sizes of 1100 nucleotides (nt) and 900 nt (FIG. 4). IVT of linearized GFP plasmid resulted in RNA products with approximate sizes of 1300 nt and 900 nt. The larger product is consistent with the expected full-length transcript for linearized GFP plasmid which is 200 bp longer than the PCR-generated linear duplex, dsT7EGFP. Denaturing PAGE ruled out the possibility that the 900 nt product was a stable folded (i.e., internally base-paired) form of the 1100 nt long product. Previous studies have shown that sequence 5′ ATCTGTT 3′ can be responsible for the pausing or termination of T7 RNA polymerase (Lyakhov et al., “Pausing and Termination by Bacteriophage T7 RNA Polymerase,” J. Mol. Biol. 280(2):201-213 (1998) and Song and Kang, “Sequence-Specific Termination by T7 RNA Polymerase Requires Formation of Paused Conformation prior to the point of RNA Release,” Genes Cells 6(4):291-301 (2001), which are hereby incorporated by reference in their entirety). The target DNA in these studies contains this sequence near the 3′ end and termination at this position would create an RNA product of the observed size. Thus, it is likely that the aforementioned termination sequence is responsible for the smaller of the two different RNA products observed. The electrophoretic results also revealed bands at approximately 2.5 kb in general and 1.5 kb in the T7GHL-T7 lane. Given that the templates for the IVT reaction did not exceed 2.5 kb (i.e., the 2500 nt product cannot be an RNA product because the templates were much shorter than 2 kb) and the ssRNA ladder used in FIG. 4 was not a dependable standard for measuring the size of dsDNA, it is reasonable to conclude that the bands around 2.5 kb represent residual DNA templates (FIG. 4).

Based on previous reports involving the study of the requirements of a duplex promoter for transcriptional activity, it was assumed that a fully single-stranded promoter would preclude transcription initiation (Arnaud-Barbe et al., “Transcription of RNA Templates by T7 RNA Polymerase,” Nucleic Acids Res. 26(15):3550-3554 (1998) and Sarear and Miller, “A Specific, Promoter-Independent Activity of T7 RNA Polymerase Suggests a General Model for DNA/RNA Editing in Single Subunit RNA Polymerases,” Sci. Rep. 8(1): 13885 (2018), which are hereby incorporated by reference in their entirety). However, ssT7EGFP, the fully single-stranded antisense scaffold, was transcribed and produced RNA products that were indistinguishable from those produced by the positive controls. Sequence analysis revealed that portions of ssT7EGFP contain partial sequence complementarity to the promoter region. Previous studies have shown that partial complementarity of this nature is sufficient to support T7 promoter function (Újvári and Martin, “Identification of a Minimal Binding Element within the T7 RNA Polymerase Promoter,” J. Mol. Biol. 273(4):775-781 (1997), which is hereby incorporated by reference in its entirety). Thus, it is hypothesized that a foldback structure mediated by partial T7 promoter complementarity permitted low-level transcription initiation with purely single-stranded template DNA.

Electrophoretic analysis of IVT products from the structured DNA nanoparticles used in this study revealed banding patterns identical to the positive controls. All of the DNA nanoparticles tested produced identically sized RNA products except for the negative control DNA nanoparticle that was completely devoid of the T7 promoter element (FIG. 4). To assess whether DNA nanoparticles retain their structural integrity under IVT reaction conditions, T7GHL FS was incubated under IVT reaction conditions in the absence of T7 RNA polymerase and in the presence of increasing magnesium ion concentration (Mg²⁺; known to enhance the formation and stability of DNA origami nanoparticles (Rothemund P W K, “Folding DNA to Create Nanoscale Shapes and Patterns,” Nature 440:297-302 (2006), which is hereby incorporated by reference in its entirety). The result was analyzed by gel electrophoresis. The banding pattern revealed no obvious change in DNA nanoparticle migration upon increasing Mg²⁺ concentration suggesting that the DNA nanoparticles used in this study maintain their structural integrity under the conditions used for IVT reactions (FIG. 8). Moreover, an inverse correlation between “compactness” (i.e., crossover density) of the DNA nanoparticles and RNA production was observed. This strengthens the suggestion that the DNA nanoparticles remain intact under T7 RNA polymerase reaction conditions and that T7 RNA polymerase, though capable of transcribing through crossovers, is increasingly impeded as the number of crossovers increases.

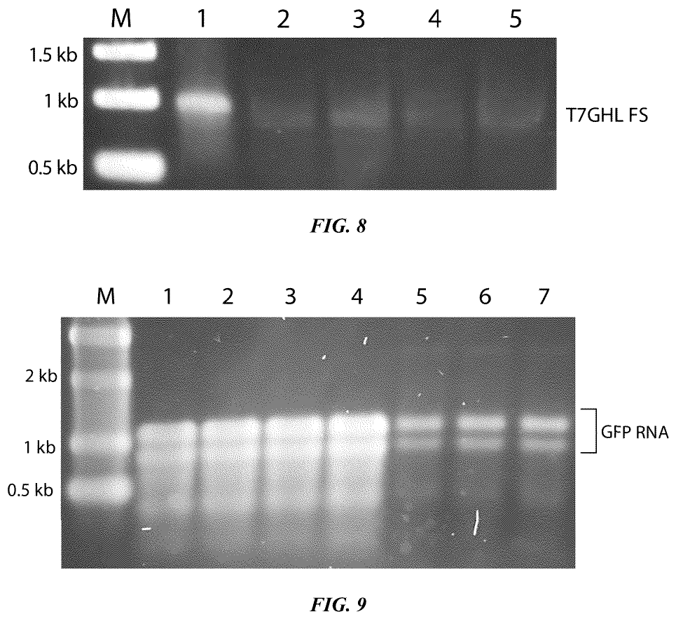

T7GHL BP (buried promoter) was designed to test whether impeding access to the promoter by positioning it in an internal region of a DNA nanoparticle can block transcription. In contrast to expectations, this architecture supported RNA production, but at a slightly reduced level in comparison to the other constructs (FIG. 4). As mentioned above, DNA nanoparticles were observed to maintain structural integrity under IVT reaction conditions as assessed by gel electrophoresis. Yet, to fully exclude the possibility of a “loosened” structure permitting access to the promoter to the T7 RNA polymerase, various concentrations of magnesium ions were introduced into the IVT reaction (FIG. 9). The production of RNA was not impacted by the increase in magnesium ion concentration, contradicting the hypothesis that a loosened architecture was the primary reason the buried promoter architecture still permitted transcription. The crystal structure of T7 RNA polymerase reveals its overall dimensions to be 75 Å× 75 Å× 65 Å (Sousa et al., “Crystal Structure of Bacteriophage T7 RNA Polymerase at 3.3 Å Resolution,” Nature 364:593-599 (1993), which is hereby incorporated by reference in its entirety). The most compact DNA nanoparticle architecture used in this study, T7GHL BP, retains at least 21 bases between any two crossovers connecting adjacent helices, corresponding to a distance of 71.4 A. Thus, it is reasonable to hypothesize that the T7 RNA polymerase single-subunit enzyme is capable of accessing the T7 promoter inside the structure.

### Example 3-RT PCR

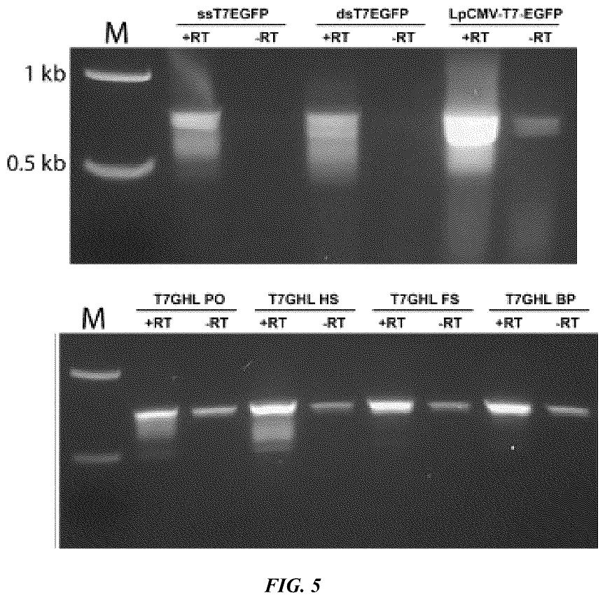

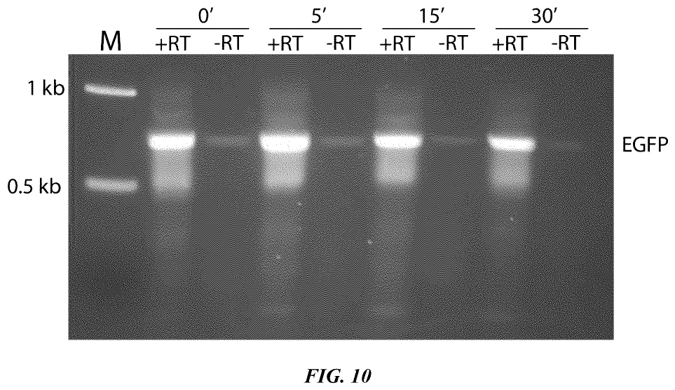

To verify that the observed IVT products were indeed GFP RNA transcripts, RT-PCR was performed. Primers were designed to cover the GFP gene. For these experiments, the amount of template used for IVT was titrated to minimize the impact of residual DNA template known to persist even after DNase treatment. To verify that the product of RT-PCR was the result of the amplification of the reverse transcribed DNA, rather than the residual DNA template, identical PCR mixtures were prepared in the absence of reverse transcriptase (negative controls). RT-PCR resulted in DNA bands of approximately 700 bp for all IVT products, which was the expected size for the GFP gene (FIG. 5). Identically sized bands appeared in the control samples, but at a substantially reduced quantity, consistent with them resulting from amplification of residual input DNA. To investigate this further, a DNase time course experiment was performed and revealed residual full-length DNA persisted after prolonged DNase treatment (FIG. 10). Thus, it was concluded that the DNA bands of approximately 700 bp in length are bona fide products of reverse transcription of RNA generated in the IVT reactions.

### Example 4-IVT Time Course

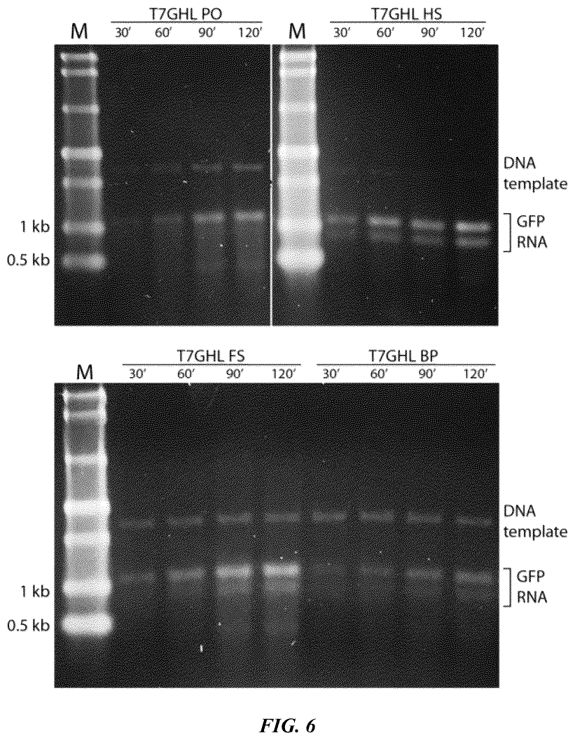

To assess the persistence/longevity of DNA nanoparticles in IVT reactions, a time course experiment was performed on the various DNA nanoparticle constructs. IVT products were extracted at 30, 60, 90, and 120 minutes of reaction time. An increase in RNA production was observed with all four promoter-bearing constructs, including the buried promoter, with no obvious variation in transcription kinetics. Thus, it appears that DNA nanoparticles remain transcription competent for at least 120 minutes in a controlled in vitro environment (FIG. 6).

### Discussion of Examples 1-4

The self-assembly nanoengineering code embedded in nucleic acids has resulted in the production of DNA nanoparticles with myriad attributes (Kearney et al., “DNA Origami: Folded DNA-Nanodevices that can Direct and Interpret Cell Behavior,” Adv. Mater. 28(27):5509-5524 (2016); Wang et al., “The Beauty and Utility of DNA Origami,” Chem. 2(3):359-382 (2017); and Rothemund P W K., “Folding DNA to Create Nanoscale Shapes and Patterns,” Nature. 440(7082):297-302 (2006), which are hereby incorporated by reference in their entirety) and, in some instances, their implementation in various applications (Kearney et al., “DNA Origami: Folded DNA-Nanodevices That Can Direct and Interpret Cell Behavior,” Advanced Materials 28(27):5509-5524 (2016); Ora et al., “Cellular Delivery of Enzyme-Loaded DNA Origami,” Chemical Communications 52(98): 14161-14164 (2016); Ramakrishnan et al., “Structural Stability of DNA Origami Nanostructures under Application-Specific Conditions,” Computat. Struct. Biotechnol. J. 16:342-349 (2018); Wang et al., “DNA Origami Protection and Molecular Interfacing through Engineered Sequence-Defined Peptoids,” PNAS 117(12):6339-6348 (2020); Daljit et al., “Switchable DNA-Origami Nanostructures that Respond to their Environment and their Applications,” Biophys. Rev. 10(5): 1283-1293 (2018); Wang et al., “The Beauty and Utility of DNA Origami,” Chem. 2:359-382 (2017); Li et al., “A DNA Nanorobot Functions as a Cancer Therapeutic in Response to a Molecular Trigger In Vivo,” Nat. Biotechnol. 36:258-264 (2018); Dobrovolskaia and Bathe, “Opportunities and Challenges for the Clinical Translation of Structured DNA Assemblies as Gene Therapeutic Delivery and Vaccine Vectors,” Wiley Interdiscip. Rev. 13(1):e1657 (2021); Ouyang et al., “Rolling Circle Amplification-Based DNA Origami Nanostructures for Intracellular Delivery of Immunostimulatory Drugs,” Small 9(18):3082-3087 (2103); Burns et al., “DNA Origami Inside-Out Viruses,” ACS Synth. Biol. 7(3):767-773 (2018); Brglez et al., “Designed Intercalators for Modification of DNA Origami Surface Properties,” Chem. Eur. J. 21(26):9440-9446 (2015); Chi et al., “DNA Nanostructure as an Efficient Drug Delivery Platform for Immunotherapy,” Front Pharmacol. 10:1585 (2020); Madsen and Gothelf, “Chemistries for DNA Nanotechnology,” Chem. Rev. 119(10):6384-6458 (2019); and Hannewald et al., “DNA Origami Meets Polymers: A Powerful Tool for the Design of Defined Nanostructures,” Angew. Chem. Int. Ed. 60(12):6218-6229 (2021), which are hereby incorporated by reference in their entirety). The applicability of DNA nanoparticles can be further amplified by the integration of expressible genes in their architecture. This latter point is reflected in a recent report in which a gene cassette folded into a DNA origami nanoparticle was utilized as the substrate for insertion at a genetic locus by a CRISPR/Cas9 editing system in cell culture. In that work, the inserted element was transcribed subsequently to linearization and insertion into the target chromosomal locus (Lin-Shiao et al., “CRISPR-Cas9-Mediated Nuclear Transport and Genomic Integration of Nanostructured Genes in Human Primary Cells,” Nucleic Acids Res. 50(3): 1256-1268 (2022), which is hereby incorporated by reference in its entirety). Furthermore, it was recently shown that DNA origami nanoparticles constructed using both sense and antisense gene-bearing scaffold strands can be expressed in cell culture (Kretzmann et al., “Gene-Encoding DNA Origami for Mammalian Cell Expression,” Nat. Commun. 14(1): 1017 (2023), which is hereby incorporated by reference in its entirety).

Examples 1˜4 explored the possibility that expression can occur within the context of folded DNA nanoparticles that include a significant number of crossovers stercochemically analogous to Holiday junctions. The ability of a polymerase to navigate past a small number of Holliday junctions in linear duplex (i.e., not folded) DNA has been previously described (Pipathsouk et al., “When Transcription Goes on Holliday: Double Holliday Junctions Block RNA Polymerase II Transcription In Vitro,” Biochem. Biophys. Acta. 1860(2):282-288 (2017), which is hereby incorporated by reference in its entirety). Examples 1˜4 demonstrate that Holliday junctions impede, but do not completely block polymerase progression. These results demonstrate that even under conditions of extreme folding, high crossover density, and limited accessibility to the promoter, T7 RNA polymerase can initiate and complete the transcription of a full-length gene cassette. Kretzmann and colleagues have proposed that the unfolding of DNA nanoparticles is a prerequisite for gene expression in cell culture (Kretzmann et al., “Gene-Encoding DNA Origami for Mammalian Cell Expression,” Nat. Commun. 14(1): 1017 (2023), which is hereby incorporated by reference in its entirety). The results presented herein suggests that, at least in vitro, DNA nanoparticles retain their structure prior to transcription and that the unfolding of the DNA nanoparticles occurs during the transcription event. This variation in the proposed mechanism is supported by the observed trend in differences in RNA production as a function of origami “compactness” (i.e., number, and local density of crossovers). Further delineation of the details involved in gene expression from sculpted DNA nanoparticles may permit regulation of the timing and level of gene expression via fine-tuning of their architectural features.

It is noteworthy that the chemical malleability of DNA nanoparticles allows them to be readily decorated with a variety of molecular and chemical species, offering mechanisms for enhanced biocompatibility and cellular targeting. For example, electrostatic coating of lipid nanoparticles can significantly enhance their in vivo stability (Wang et al., “DNA Origami Protection and Molecular Interfacing through Engineered Sequence-Defined Peptoids,” PNAS 117(12):6339-6348 (2020); Dobrovolskaia and Bathe, “Opportunities and Challenges for the Clinical Translation of Structured DNA Assemblies as Gene Therapeutic Delivery and Vaccine Vectors,” Wiley Interdisciplinary Rev. Nanomed. Nanobiotechnol. 13(1):c 1657 (2021); and Agarwal et al., “Block Copolymer Micellization as Protection Strategy for DNA Origami,” Angew. Chem. Int. Ed. Engl. 56(20):5460-5464 (2017), which are hereby incorporated by reference in their entirety), various cell-penetrating peptides, as well as nuclear localization signals, can be employed through either conjugation or intercalation for targeted delivery to the nucleus for efficient subsequent gene expression (Burns et al., “DNA Origami Inside-Out Viruses,” ACS Synth. Biol. 7(3): 767-773 (2018); Bolhassani et al., “In vitro and in vivo Delivery of Therapeutic Proteins Using Cell Penetrating Peptides,” Peptides 87:50-63 (2017); Bogacheva et al., “Arginine-Rich Cross-Linking Peptides with Different SV40 Nuclear Localization Signal Content as Vectors for Intranuclear DNA Delivery,” Bioorg. Med. Chem. Lett. 27:4781-4785 (2017); Zanta et al., “Gene Delivery: A Single Nuclear Localization Signal Peptide is Sufficient to Carry DNA to the Cell Nucleus,” PNAS 96(1):91-96 (1999); and Vinogradov et al., “Intercalating Conjugates of PEG with Nuclear Localization Signal (NLS) Peptide,” Polym. Prepr. 49(2):434-435 (2008), which are hereby incorporated by reference in their entirety), and antigen-specific aptamers can be utilized for specific cell targeting (Li et al., “A DNA Nanorobot Functions as a Cancer Therapeutic in Response to a Molecular Trigger In Vivo,” Nat. Biotechnol. 36:258-264 (2018), which is hereby incorporated by reference in its entirety). Thus, self-assembling, gene-bearing DNA nanoparticles may constitute the centerpiece of a broadly applicable platform for targeted in vivo delivery of therapeutic proteins (Dimitrov D S., “Therapeutic Proteins,” Methods Mol. Biol. 899:1-26 (2012); El Taher and Yaqeen, “Therapeutic Proteins Derived from Recombinant DNA Technology,” Int. J. Curr. Microbiol. App. Sci. 9(1):2024-2032 (2020); and Leader et al., “Protein Therapeutics: A Summary and Pharmacological Classification,” Nat. Rev. Drug Discov. 7(1):21-39 (2008), which are hereby incorporated by reference in their entirety), protein vaccines (Dimitrov D S., “Therapeutic Proteins,” Methods Mol. Biol. 899:1-26 (2012); El Taher and Yaqeen, “Therapeutic Proteins Derived from Recombinant DNA Technology,” Int. J. Curr. Microbiol. App. Sci. 9(1):2024-2032 (2020); Leader et al., “Protein Therapeutics: A Summary and Pharmacological Classification,” Nat. Rev. Drug Discov. 7(1):21-39 (2008); and Pollet et al., “Recombinant Protein Vaccines, A Proven Approach Against Coronavirus Pandemics,” Adv. Drug Deliv. Rev. 170:71-82 (2021), which are hereby incorporated by reference in their entirety), and gene editing systems (Wei et al., “Systemic Nanoparticle Delivery of CRISPR-Cas9 Ribonucleoproteins for Effective Tissue Specific Genome Editing,” Nat. Commun. 11(1):3232 (2020); Kuhn et al., “Delivery of Cas9/sgRNA Ribonucleoprotein Complexes via Hydroxystearyl Oligoamino Amides,” Bioconj. Chem. 31(3):729-742 (2020); Li et al., “Non-Viral Delivery Systems for CRISPR/Cas9-Based Genome Editing: Challenges and Opportunities,” Biomaterials 171:207-218 (2018); Behr et al., “In Vivo Delivery of CRISPR-Cas9 Therapeutics: Progress and Challenges,” Acta Pharm. Sin. B. 11(8):2150-2171 (2021); and Chen et al., “A Biodegradable Nanocapsule Delivers a Cas9 Ribonucleoprotein Complex for In Vivo Genome Editing,” Nat. Nanotechnol. 14:974-980 (2019), which are hereby incorporated by reference in their entirety).

Examples 1˜4 demonstrate that gene-bearing, self-assembling DNA nanoparticles formed by the method of DNA origami and containing a large number of crossover domains can be readily transcribed in vitro by T7 RNA polymerase. Crossover density (compactness) and architecturally controlled accessibility to the T7 promoter element can modulate transcription production. In conjunction with previous studies, these results further illustrate the potential of sculpted, gene-bearing, self-assembling nucleic acid nanoparticles to offer significant value in biomedicine and related areas.

### Background for Examples 5-6

Targeted delivery of biomaterials in vivo has significantly improved therapeutic agent efficacy and safety in biomedicine by increasing accumulation at specific target sites and reducing off-target effects (Harada et al., “Antitumor Protein Therapy: Application of the Protein Transduction Domain to the Development of a Protein Drug for Cancer Treatment,” Breast Cancer 13: 16-26 (2006); Marchetti et al., “Targeted Drug Delivery via Caveolae-Associated Protein PV1 Improves Lung Fibrosis,” Commun Biol 2:92 (2019); Yan et al., “Targeted and Intracellular Delivery of Protein Therapeutics by a Boronated Polymer for the Treatment of Bone Tumors,” Bioact Mater 7:333-340 (2022); Rosenbaum et al., “Targeting Receptor Complexes: A New Dimension in Drug Discovery,” Nature Reviews Drug Discovery 19(12):884-901 (2020); and Zhao et al., “Targeting Strategies for Tissue-Specific Drug Delivery,” Cell 181(1): 151-167 (2020), which are hereby incorporated by reference in their entirety). Currently, various molecules, as well as application approaches, are being explored to target specific tissues, cells, and even subcellular compartments in vivo (Marchetti et al., “Targeted Drug Delivery via Caveolae-Associated Protein PV1 Improves Lung Fibrosis,” Commun Biol 2:92 (2019); Yan et al., “Targeted and Intracellular Delivery of Protein Therapeutics by a Boronated Polymer for the Treatment of Bone Tumors,” Bioact. Mater. 7:333-340 (2022); Zhao et al., “Targeting Strategies for Tissue-Specific Drug Delivery,” Cell 181(1): 151-167 (2020); Brandén et al., “A Peptide Nucleic Acid-Nuclear Localization Signal Fusion that Mediates Nuclear Transport of DNA,” Nat. Biotechnol. 17:784-787 (1999); Iwasaki et al., “A Polylysine-Polyhistidine Fusion Peptide for Lysosome-Targeted Protein Delivery,” Biochem. Biophys. Res. Commun. 533:905-912 (2020); Spencer and Verma, “Targeted Delivery of Proteins Across the Blood-Brain Barrier,” PNAS 104(18): 7594-7599 (2007); Liang et al., “Targeted Intracellular Protein Delivery Based on Hyaluronic Acid-Green Tea Catechin Nanogels,” Acta Biomater. 33:142-152 (2016); and Schneider et al., “Targeted Subcellular Protein Delivery Using Cleavable Cyclic Cell-Penetrating Peptides,” Bioconjug. Chem. 30:400-404 (2019), which are hereby incorporated by reference in their entirety). A promising strategy for the targeted delivery of nucleic acid biomedicines is the use of self-assembling DNA nanoparticles constructed by the method of DNA origami and related techniques (Rothemund P W K, “Folding DNA to Create Nanoscale Shapes and Patterns,” Nature 440(7082):297-302 (2006) and Nieves et al., “DNA-Based Super-Resolution Microscopy: DNA-PAINT,” Genes (Basel) 9(12):621 (2018), which are hereby incorporated by reference in their entirety). This approach provides both chemical and architectural flexibility, thereby establishing a platform for precise control over the nanoscale molecular arrangement of targeting agents for nanoparticle drug delivery vehicles (Mela et al., “DNA Nanostructures for Targeted Antimicrobial Delivery,” Angewandte Chemie-International Edition 59:12698-12702 (2020); Ge et al., “DNA Origami-Enabled Engineering of Ligand-Drug Conjugates for Targeted Drug Delivery,” Small 16(16):c 1904857 (2020); Pal and Rakshit, “Folate-Functionalized DNA Origami for Targeted Delivery of Doxorubicin to Triple-Negative Breast Cancer,” Front. Chem. 9:721105 (2021); Zhang et al., “DNA Origami as an In Vivo Drug Delivery Vehicle for Cancer Therapy,” ACS Nano 8:6633-6643 (2014); and Ma et al., “The Biological Applications of DNA Nanomaterials: Current Challenges and Future Directions,” Signal Transduct. Target. Ther. 6(1):351 (2021), which are hereby incorporated by reference in their entirety). Currently, a wide range of site-specifying conjugates, attachment strategies, and active switching mechanisms are being explored to fully leverage the DNA nanoparticle delivery platform for biomedicine (Ma et al., “The Biological Applications of DNA Nanomaterials: Current Challenges and Future Directions,” Signal Transduct. Target. Ther. 6(1):351 (2021); Knappe et al., “Functionalizing DNA Origami to Investigate and Interact with Biological Systems,” Nat. Rev. Mater. 8(2):123-138 (2022); and Daljit et al., “Switchable DNA-Origami Nanostructures that Respond to their Environment and their Applications,” Biophys. Rev. 10(5): 1283-1293 (2018), which are hereby incorporated by reference in their entirety). Nonetheless, to date, self-assembled DNA nanoparticles have primarily been envisioned as delivery vessels with internal or attached molecular cargo. However, an additional dimension has recently been added to the potential utility of sculpted DNA nanoparticles wherein the particle itself, constructed from biologically active genetic elements, is both the carrier and the cargo (Kretzmann et al., “Gene-Encoding DNA Origami for Mammalian Cell Expression,” Nat. Commun. 14(1): 1017 (2023); Lin-Shiao et al., “CRISPR-Cas9-Mediated Nuclear Transport and Genomic Integration of Nanostructured Genes in Human Primary Cells,” Nucleic Acids Res. 50(3): 1256-1268 (2022); and Wu et al., “Genetically Encoded DNA Origami for Gene Therapy In Vivo,” J. Am. Chem. Soc. 145:9343-9353 (2023), which is hereby incorporated by reference in its entirety). Lin-Shiao et al. used a DNA origami nanoparticle as a CRISPR-mediated insertion element in cell culture (Lin-Shiao et al., “CRISPR-Cas9-Mediated Nuclear Transport and Genomic Integration of Nanostructured Genes in Human Primary Cells,” Nucleic Acids Res. 50(3): 1256-1268 (2022), which is hereby incorporated by reference in its entirety), and Kretzmann et al. demonstrated the transcription of fully folded DNA nanostructures in cell culture, showcasing the cells' versatile capability to express genes embedded in sculpted DNA nanoparticles (Kretzmann et al., “Gene-Encoding DNA Origami for Mammalian Cell Expression,” Nat. Commun. 14(1): 1017 (2023), which is hereby incorporated by reference in its entirety). Interestingly, in the latter case gene expression was achieved when utilizing either the sense or antisense strands as the nanoparticle scaffold, suggesting that DNA origami nanoparticles are capable of supporting both DNA replication and transcription in the cellular milieu (Kretzmann et al., “Gene-Encoding DNA Origami for Mammalian Cell Expression,” Nat. Commun. 14(1): 1017 (2023), which is hereby incorporated by reference in its entirety).

Examples 5 and 6 below focus on architectural details involved in DNA nanoparticle gene expression in human cell culture, using, in part, nanoparticles comprising identical staple sequences as shown in Table 4 supra as well as additional constructs. Thee nanoparticles were evaluated in an in vitro transcription system. In general, folded DNA nanoparticles tend to be more resistant to nuclease degradation than their linear counterparts, presumably owing to the former's compact architecture (Chandrasekaran A R., “Nuclease Resistance of DNA Nanostructures,” Nat. Rev. Chem. 5:225-239 (2021), which is hereby incorporated by reference in its entirety). However, these nanoparticles necessarily include nicks in duplex domains at juxtaposed staples and, often, single-stranded DNA (ssDNA) domains to facilitate folding, both of which are highly susceptible to nuclease degradation. A single scission at a ssDNA domain could render a folded DNA nanoparticle genetically inactive while still retaining its overall architecture. Thus, it may be possible to modulate DNA nanoparticle efficacy in cell culture by varying the double-stranded DNA (dsDNA) to ssDNA ratio as well as the level of compactness adjusted by the ratio. Furthermore, folding strategies implemented to date for gene expression in cell culture collectively incorporate folded enhancers, promoters, and genes (Kretzmann et al., “Gene-Encoding DNA Origami for Mammalian Cell Expression,” Nat. Commun. 14(1): 1017 (2023); Lin-Shiao et al., “CRISPR-Cas9-Mediated Nuclear Transport and Genomic Integration of Nanostructured Genes in Human Primary Cells,” Nucleic Acids Res. 50(3): 1256-1268 (2022); Wu et al., “Genetically Encoded DNA Origami for Gene Therapy In Vivo,” J. Am. Chem. Soc. 145:9343-9353 (2023); and Liedl et al., “Active Nuclear Import of Mammalian Cell-Expressible DNA Origami,” J. Am. Chem. Soc. 145:4946-4950 (2023), which is hereby incorporated by reference in its entirety). As such, the distinct contributions of these individual components toward gene expression with respect to their duplex/single-stranded disposition and/or compactness warrants further investigation. Examples 5 and 6 assess the significance of local ssDNA domains on gene expression of DNA origami nanoparticles in cell culture and examine the independent contributions of each gene expression element (i.e., enhancer, promoter, and gene) when folded separately.

### Materials and Methods for Examples 5 and 6

Gene-bearing DNA origami scaffolds were prepared as previously described (Oh and Henderson, “A Comparison of Methods for the Production of Kilobase-Length Single-Stranded DNA,” DNA 2:56-67 (2022), which is hereby incorporated by reference in its entirety) and described briefly below.

### Standard PCR

Primers for amplification of the Green Fluorescent Protein (GFP) gene were designed using SnapGene and purchased from Integrated DNA Technologies (IDT, Coralville, IA). Sequences of each primer are listed in Table 8. Phusion® DNA polymerase for the PCR reaction was purchased from New England Biolabs (NEB, Ipswich, MA). Each PCR reaction mixture was prepared in 50 μl final volume, composed of 1×Phusion® HF buffer (NEB), 200 nM dNTP mix (NEB), 500 nM sense primer (EGFP sense=undesired strand), 500 nM antisense primer (EGFP anti=desired strand), 10 ng plasmid template (pCMV-T7-EGFP; Addgene, Watertown, MA, USA), 0.5 μl Phusion® DNA polymerase, and nuclease-free water to volume. Each PCR was performed using the following thermocycler steps: 30 s at 98° C., 30 s at 58° C., and 1 min at 72° C. for 30 cycles. The reaction product was mixed with NEB 6×loading dye (15% Ficoll®-400, 60 mM EDTA, 19.8 mM Tris-HCl, 0.48% SDS, 0.12% Dye 1, 0.006% Dye 2, pH 8 at 25° C.; NEB) and then loaded onto a 1% agarose gel pre-stained with SYBR safe DNA dye (Invitrogen, Waltham, MA, USA).

Electrophoresis was carried out at 8V/cm for 1 hour. The SYBR Safe-containing DNA was visualized using a 490 nm wavelength (blue) transilluminator and an amber filter.

TABLE 8
Primer Sequences for PCR and Asymmetric PCR (aPCR)

| Primer | Sequences | SEQ ID NO: |
|---|---|---|
| GFP sense | 5′ CCA GAT ATA CGC GTT GAC ATT GAT TAT TGA 3′ | 30 |
| GFP anti | 5′ TAT ATA CAA TTT CAC ACA GGA AAC AGC TAT GA 3′ | 31 |
| 3′ GFP | 5′ CCA GAT ATA CGC GTT GAC ATT GAT TAT TGA/3′phos/3′ | 32 |
| blocker | | |

Modifications: /3′phos/: 3′ phosphorylation

Asymmetric PCR (aPCR)

Primers used in aPCR were identical to those used in standard PCR. Along with sense and antisense primers, 3′ terminal modified primer (3′ EGFP blocker) was used. 3′ EGFP blocker was designed using SnapGene and purchased from IDT. Its sequence is listed in Table 8. For the generation of a scaffold, each aPCR reaction was carried out in 50 μL total volume, composed of 1×LongAmp® Taq buffer (60 mM Tris-SO4, 20 mM (NH4)₂SO4, 2 mM MgSO4, 3% glycerol, 0.06% IGEPAL® CA-630, 0.05% Tween® 20, pH 9.1 at 25° C.) from NEB, 500 nM EGFP anti, 25 nM EGFP sense, 475 nM 3′ EGFP blocker, 300 nM dNTP mix from NEB, 10 ng double-stranded GFP gene (dsEGFP) (generated by standard PCR), 2 μL LongAmp® Taq DNA polymerase (NEB), and nuclease-free water to final volume. Each aPCR was performed using the following thermocycler steps: 30 seconds at 94° C., 30 seconds at 59° C., and 2 minutes at 65° C. for 25 cycles. The reaction product was loaded onto a 1% agarose gel pre-stained with 1×SYBR Safe (Invitrogen), electrophoresed, and visualized as above.

### DNA Purification

Double-stranded DNA (dsDNA) purification. A Zymoclean Gel DNA Recovery Kit (Zymo Research, Irvine, CA) was used to extract dsDNA from agarose gels. Gel bands containing target dsDNA were removed using a clean razor blade. Three times the gel slice volume of the provided agarose dissolving/binding buffer was added to each sliced gel fragment and incubated at 55° C. on a heating block for 15 minutes. Each dissolved gel solution was transferred to the provided silica-based spin columns and centrifuged at 10,000 relative centrifugal force (rcf) for 60 seconds in a table-top centrifuge. 200 μL of ethanol-based DNA wash buffer was added to each spin column and centrifuged at 10,000 rcf for 30 seconds. A washing step was repeated before centrifuging at 10,000 rcf for 60 seconds for the complete removal of ethanol. Flow-through from all steps was discarded. After transferring each spin column to a clean microcentrifuge tube, 6-20 μL of the provided elution buffer (10 mM Tris-HCl, 0.1 mM EDTA, pH 8.5) was added directly to the matrix of each spin column followed by centrifugation at 10,000 rcf for 60 seconds for DNA collection. A fraction of each purified dsDNA was mixed with 6×loading dye (NEB) and loaded onto 1% agarose gel pre-stained with 1×SYBR safe. The gel was run at 8V/cm for 1 hour. The yield of the purified dsDNA samples was evaluated by measuring band intensities relative to a known control using GelAnalyzer 19.1 available at www.gelanalyzer.com.

Single-stranded (ssDNA) purification. A Zymoclean Gel RNA Recovery Kit from Zymo Research was used to purify ssDNA from agarose gels. The gel bands containing target ssDNA were excised with a clean razor blade. Three times the gel slice volume of the provided agarose dissolving/binding buffer were added to each excised gel band and melted at 55° C. on a heating block for 15 minutes. Each dissolved gel solution was transferred to a provided silica-based spin columns and centrifuged at 12,000 rcf for 2 minutes. 400 μL RNA Prep buffer was added to each spin column followed by centrifugation at 12,000 ref for 1 minute. Washing was carried out by the addition of 800 L ethanol-based wash buffer followed by centrifugation at 12,000 rcf for 30 seconds. After repeating the washing step with 400 μL ethanol-based wash buffer, each spin column was centrifuged at 12,000 rcf for 2 minutes to remove residual ethanol. Flow-through in all steps was discarded. After transferring each spin column to clean microcentrifuge tubes, 6-20 μL of provided nuclease-free water was added directly to the column matrix, and the spin columns were centrifuged at 10,000 rcf for 1 minute for retentate collection. A fraction of each purified ssDNA was mixed with 6×loading dye (NEB) and the yield was estimated by gel electrophoresis as described above.

### DNA Nanoparticle Construction

The DNA nanoparticles were designed using caDNAno (www.cadnano.org) and staples were purchased from IDT (Table 9). DNA nanoparticles were prepared by mixing single-stranded GFP gene (ssGFP; generated by aPCR) to a final concentration of 91.4 nM and each staple to a final concentration of 731.2 nM in 1×TAE buffer supplemented with 12.5 mM Mg(OAc)₂ in a final volume of 50 μL (TAEM). The staple set used for each DNA nanoparticle is listed in Table 10. The mixture was incubated at 90° C. for 10 minutes in a water bath followed by gradual cooling to room temperature. After the construction, DNA nanoparticles were purified through Polyethylene glycol (PEG) precipitation method and ultrafiltration. For ultrafiltration, the final volume of each DNA nanoparticle was scaled up to 500 μL by adding 1×Phosphate-buffered saline containing 12.5 mM Mg(OAc)₂ (PBSM). For the purification of final products, excess staples were removed using an Amicon ultracentrifugation filter with a 100 kDa molecular cutoff (Millipore Sigma, Burlington, MA, USA). 500 μL DNA nanoparticle product was applied to each filter unit followed by centrifugation at 14,000 ref for 3 minutes. This step was repeated three times with the addition of 400 μL of 1×PBSM to ensure the full removal of staples. All the flowthroughs were discarded. For the elution of purified DNA nanoparticles, the centrifugation was done at 14,000 rcf for 5 minutes with the filter unit inverted. For PEG precipitation, each DNA nanoparticle was mixed with an equal volume of PEG precipitation buffer (17.5% PEG 8000 (Sigma), 500 mM NaCl, and 10 mM MgCl2). After the incubation in 4° C. for 10 minutes, the mixture was centrifuged at 16,000 rcf for 25 minutes, followed by carefully removing supernatant through a pipette. The pellet was incubated in 30-50 μL 1×PBSM in room temperature for overnight for resuspension. The purified products were verified by mixing with 6×loading dye (NEB) and then loading onto 1% agarose gel containing 12.5 mM Mg(OAc)₂. Electrophoresis was carried out in TAEM buffer at 6V/cm for 90 minutes. The gel was post-stained with 1×SYBR gold DNA dye (Invitrogen) and visualized as above. The quantity of each purified DNA nanoparticle was measured by measuring the quantities of the scaffold. A fraction of each purified DNA nanoparticle (2 μL) was mixed with 18 μL nuclease-free water followed by heating the samples at 70° C. for 10 minutes to purposely disintegrate the nanoparticle (i.e., remove staples). The heated mixtures were mixed with 6×loading dye (NEB) and were gel electrophoresed on 1% agarose gel pre-strained with 1×SYBR safe (Invitrogen) as described above. The quantities were measured using GelAnalyzer 19.1 as described above.

TABLE 9
DNA Nanoparticle (GHL and GHH) Staple Sequences

| Staple Names | Sequences | SEQ ID NO: |
|---|---|---|
| GHL 1 | AAG CTG GAT CCG CCC CTG AGC ACC CAG TGT TCG TG | 35 |
| GHL 2 | AGT TCC GCG TTA CAT AAC TTA CGG TAA ATG GCC CGC | 36 |
| | CTG GCT | |
| GHL 3 | GCT GCC CCC ACC CTC GTG ACC GGG CGA C | 37 |
| GHL 4 | AGG TGC CGC CAT CTT GCT GGA CCG CCC TAG GTG AA | 38 |
| GHL 5 | ATG GGT GGA GTA TTT ACG GTA AAC TGC CCA CTT GGC | 39 |
| | AGT ACA | |
| GHL 6 | CGG TAG GCG TGT ACG GTG GGA GGT C | 40 |
| GHL 7 | CGC CGA CTC GAC TTA GTT CGA ACC CTG ATG CAC CA | 41 |
| GHL 8 | ATC GCT ATT ACC ATG GTG ATG CGG TTT TGG CAG TAC | 42 |
| GHL 9 | CGA GGA CAC ATC CTA CAA GAC TGC TTC AAA GCT GA | 43 |
| GHL 10 | AGG ACA GCT GTT TCC TGT GTG GCA AGG GCG AGG | 44 |
| | AGG TTC ATC CCT ACG G | |
| GHL 11 | CCA GAT ATA CGC GTT GAC ATT GAT TAT TGA CTA GTT | 45 |
| | ATT AAT | |
| GHL 12 | CCC CGA CGG ACG ACG GCA ACT GGG GCA C | 46 |
| GHL 13 | TGT TCC CAT AGT AAC GCC AAT AGG GAC TTT CCA TTG | 47 |
| | ACG TCA | |
| GHL 14 | AAT CAT GCC GGG GTG GTG CCC CTA CGG CGC CGC TA | 48 |
| GHL 15 | CTT CAA GAG TAC AAT CTT CAA CAC ATG AGA GGG CG | 49 |
| GHL 16 | GAA GGC TTA TCA TGG C | 50 |
| GHL 17 | TAT ATA AGC AGA GCT GGT TTA GTG AAC CGT CAG ATC | 51 |
| | CGC TAG | |
| GHL 18 | ATG CCA CAT CCT GGC TAG AGC CAA TAG CCT GTC CT | 52 |
| GHL 19 | CCT CTA GTC GAG CTG GAC GGC CGA GGG CAG CAG CA | 53 |
| GHL 20 | TTC CTA AGA CTG TGC CGG GAT ACC ACT AAC AAC AT | 54 |
| GHL 21 | TGT CCG GGA CGT AAC TCG ATA TGC GGT GCC CTG GA | 55 |
| GHL 22 | AGT AAT CAA TTA CGG GGT CAT TAG TTC ATA GCC CAT | 56 |
| | ATA TGG | |
| GHL 23 | AGC CAC AGG CAT CAG AGC AAA GAC CCC ACG ATC | 57 |
| | ACA TGG TCC GTT GTT T | |
| GHL 24 | GAC CGC CCA ACG ACC CCC GCC CAA TGA CGT CAA | 58 |
| | TAA TGA CGT A | |
| GHL 25 | AGA ACC AGC TGG GGA CGG CCA CA | 59 |
| GHL 26 | AGA TCC GCG GCC GCT AAT ACG ACT CAC TAT AGG | 60 |
| | GAG AGC CGC CAC C | |
| GHL 27 | TCA AGT GTA TCA TAT GCC AAG TAC GCC CCC TAT TGA | 61 |
| | CGT CAA T | |
| GHL 28 | GAC GGC AGG CAG CGG CTG CTG CCC GAC ACA CTC TC | 62 |
| GHL 29 | AGT CTC CAC CCC ATT GAC GTC AAT GGG AGT TTG TTT | 63 |
| | TGG CAC | |
| GHL 30 | CGT GCA GCC GCG CCG AGG TGA CAA GGA G | 64 |
| GHL 31 | ACA AGC AGA AGA ACA CGT CTA ACG TCC ATT CAA GTT | 65 |
| | TCA GCG | |
| GHL 32 | GAC GGT AAA TGG CCC GCC TGG CAT TAT GCC CAG TAC | 66 |
| | ATG ACC | |
| GHL 33 | ATC AAT GGG CGT GGA TAG CGG TTT GAC TCA CGG | 67 |
| | GGA TTT CCA | |
| GHL 34 | TTA TGG GAC TTT CCT ACT TGG CAG TAC ATC TAC GTA | 68 |
| | TTA GTC | |
| GHL 35 | CCA TTG ACG CAA ATG GG | 69 |
| GHL 36 | CCC TGA ACT GTT CAG TCA TAG CAA GGG GAG GAA AT | 70 |
| GHL 37 | TGC ATC GTT AAA CCA CGA GCT GCC CCG TTG CAG CT | 71 |
| GHL 38 | CGA CTT CGG AGC GCA CCA TCT CTA CAA C | 72 |
| GHL 39 | GGC ATG GCG CTG ATC AGC CTC TAA AAT GGA GGA TTG | 73 |
| | GGA AGA TTG GCG T | |
| GHL 40 | ACC CTG GCG AGC TGA AGG GCA CAC TAC CCC CCA TCG | 74 |
| | GCG ACG GTA CAA G | |
| GHL 41 | GCC CCT CTC CTT GAG GCT CTA TGG CTT CTG AGG | 75 |
| GHL 42 | CAA AAT CAA CGG GAC TTT CCA AAA TGT CGT AAC AAC | 76 |
| | TCC GCC | |
| GHL 43 | TGA CCG GCA TCA CCA TTG AGT CAT TGT CGT GGG GC | 77 |
| GHL 44 | ACC GCC GCC TTC TAG TTG CCA ACT CCC AAG GCA TGC | 78 |
| | TGG GGA CCG TCG A | |
| GHL 45 | CCG GCA AAT GGT GAA AAT TGT GGG TGG GTG AGT | 79 |
| | AGG TGT CAT | |
| GHL FSC 1 | TAT TCT GGG GGG TGG GTG AGT AGG TGT CAT TC | 129 |
| GHL FSC 2 | GCT GCC CGT GCC CTG GCC CAC CCT CGT GAC CGG GCG | 130 |
| | ACA CCC TGG TGA AC | |
| GHL FSC 3 | AAG CGC GAT CAC ATG GTC CGT TGT TTG CCC CTC CCC | 131 |
| | CG | |
| GHL FSC 4 | CCG AAG GCT TAT CAT GGC | 132 |
| GHL FSC 5 | AGT TCA GCG TGT CCG GGA CGT AAC TCG ATA TGC GGT | 133 |
| | GCC CTG GA | |
| GHL FSC 6 | CGC ATC GAG CTG AAG GGC ACA CTA CCA GCA G | 134 |
| GHL FSC 7 | AGC CAC AGG CAT CAG AGC AAA GAC CCC AAC GAG | 135 |
| GHL FSC 8 | CGG AAA GAA CCA GCT GGG GAC GGC CAC A | 136 |
| GHL FSC 9 | CCG GCA AAT GGT GAA AAT TGT ATA TA | 137 |
| GHL FSC 10 | CGA CAA GCA GAA GAA CAC GTC TAA CGT CCA TTC AAG | 138 |
| | TCC GCC ATG C | |
| GHL FSC 11 | AAC ACC CCC ATC GGC GAC GGT ACA AGT GAC CGG TCA | 139 |
| | TC | |
| GHL FSC 12 | TGC CTT CCT TGA GGC TCT ATG GCT TCT GAG G | 140 |
| GHL FSC 13 | ATC ACC ATC ACC ATT GAG TCA TTG TCG TGG GGC | 141 |
| GHH 1 | CGG GAC TTT CCA AAA TGT CGT AAC AAC | 142 |
| GHH 2 | TGA CGT CAA TAA TGA CGT ATG TTC CCA TA | 143 |
| GHH 3 | CAG TAC ATC AAT GGG CGG TGA TGC GGT TTT GG | 144 |
| GHH 4 | CAT GAC CTT ATG GGA CTT TCC CCC GCA T | 145 |
| GHH 5 | CCG CGG CGA CAT TGC CAT ATA TGG AGT T | 146 |
| GHH 6 | TGG CAT CAG TAC ATC AAG TGC AAT AGG GAC TTT CCA | 147 |
| | T | |
| GHH 7 | TAT TGA CGT CAA TGA CGG TAA ATG GCC CGC C | 148 |
| GHH 8 | GTA ACG CTA TCA TAT GCC AAG TAC GCC CCC | 149 |
| GHH 9 | ATG GGC GGT AGG CGT GTA CGG TGA CTA TAG GGA | 150 |
| | GAG CCG CCA CC | |
| GHH 10 | TGA ACA TTG ACG TCA ATG GGA GTT TGT TTT GAC GCA | 151 |
| | A | |
| GHH 11 | CCG CGT TCA GAT ATA CGC GTT CGC TAA TAC GAC TC | 152 |
| GHH 12 | TCA TCT TTA CGG TAA ACT GCC CAC TTG GTA TGC CCA | 153 |
| | GTA | |
| GHH 13 | TCC GCC CCA TTG GCA CCA AAA TCA A | 154 |
| GHH 14 | TTG ACT CAC GGG GAT TTC CAA GTC TCC ACC | 155 |
| GHH 15 | TAC TTG GCA GTA CAT CTA CGT ATT A | 156 |
| GHH 16 | TGA CGT CAA TGG GTG GAG TAA TCA C | 157 |
| GHH 17 | CAA TTA CGG GGT CAT TAG T | 158 |
| GHH 18 | GGA GGT CTA TAT AAG CAG AGC | 159 |
| GHH 19 | GTC ATC GCT ATT ACC ATG TGG ATA GCG GT | 160 |
| GHH 20 | TCA TAG CAT TAT TGA CTA GTT ATT AAT AGT AAT | 161 |
| GHH 21 | TGA CCG CCC AAC GAC CCC CGC CCA A | 162 |
| GHH 22 | ACA TAA CTT ACG GTA AAT GGC CCG CCT GGC | 163 |
| GHH 23 | CTA GAG ATT GGT TTA GTG AAC CGT CAG ATC CG | 164 |
| GHH LB 1 | TAT TCT GGG GGG TGG GGT GGG GCA GGA CAG CAA G | 165 |
| GHH LB 2 | CGC ATC GAG CTG AAG GGC ATC GAC TTC AAG GAG | 166 |
| GHH LB 3 | TCA AGG ACG ACG GCA ACT ACA AGA CCC GCG CCG | 167 |
| GHH LB 4 | TGC CTT CCT TGA CCC TGG AAG GTG CCA | 168 |
| GHH LB 5 | GGG GAG GAT TGG GAA GAC AAT AGC AGG CAT GCT GG | 169 |
| GHH LB 6 | ATG GTG AGC AAG GGC GAG GAG CTG TTC AC | 170 |
| GHH LB 7 | CGG CGT GCA GTG CTT CAG CCG CTA CCC CGA CC | 171 |
| GHH LB 8 | CGA CAA GCA GAA GAA CGG CAT CAA GGT GAA C | 172 |
| GHH LB 9 | CTG GCC CAC CCT CGT GAC CAC CCT GAC CTA | 173 |
| GHH LB 10 | ATC ACC ATC ACC ATT GAG TTT AAA CCC GCT | 174 |
| GHH LB 11 | AAG CGC GAT CAC ATG GTC CTG CTG GAG | 175 |
| GHH LB 12 | ACA TGA AGC AGC ACG ACT TCT TCA AGT CCG CCA TGC | 176 |
| GHH LB 13 | GGA CGA GCT GTA CAA GTG ACC GGT CAT C | 177 |
| GHH LB 14 | AAC ACC CCC ATC GGC GAC GGC CCC GTG CT | 178 |
| GHH LB 15 | CGT GCA GCT CGC CGA CCA CTA CCA GCA G | 179 |
| GHH LB 16 | TTC AAG ATC CGC CAC AAC ATC GAG GAC GGC AG | 180 |
| GHH LB 17 | AGG TGA AGT TCG AGG GCG ACA CCC TGG TGA AC | 181 |
| GHH LB 18 | ACT ACA ACA GCC ACA ACG TCT ATA TCA TGG C | 182 |
| GHH LB 19 | CGG AAA GAA CCA GCT GGG GCT CGA TAC CGT | 183 |
| GHH LB 20 | TTC GTG ACC GCC GCC GGG ATC ACT CTC GGC AT | 184 |
| GHH LB 21 | GGA TGC GGT GGG CTC TAT GGC TTC TGA GG | 185 |
| GHH LB 22 | CCA CCT ACG GCA AGC TGA CCC TGA AGT TCA T | 186 |
| GHH LB 23 | CTC CCA CTG TCC TTT CCT AAT AAA ATG AGG AAA TTG | 187 |
| GHH LB 24 | CAT CGC ATT GTC TGA GTA GGT GTC ATT C | 188 |
| GHH LB 25 | GAC GGC AAC ATC CTG GGG CAC AAG CTG GAG TAC A | 189 |
| GHH LB 26 | TGG ACG GCG ACG TAA ACG GCC ACA | 190 |
| GHH LB 27 | TCC GCC CTG AGC AAA GAC CCC AAC GAG | 191 |
| GHH LB 28 | CTG CAC CAC CGG CAA GCT GCC CGT GCC | 192 |
| GHH LB 29 | GCC ATC TGT TGT TTG CCC CTC CCC CG | 193 |
| GHH LB 30 | CCG AAG GCT ACG TCC AGG AGC GCA CCA TCT TCT | 194 |
| GHH LB 31 | CGA CCT CTA GCT AGA GCT TGG CGT AAT CAT GGT | 195 |
| GHH LB 32 | CAT AGC TGT TTC CTG TGT GAA ATT GTA TAT A | 196 |
| GHH LB 33 | AGT TCA GCG TGT CCG GCG AGG GCG AGG GCG ATG | 197 |
| GHH LB 34 | CGG GGT GGT GCC CAT CCT GGT CGA GC | 198 |
| GHH LB 35 | GCT GCC CGA CAA CCA CTA CCT GAG CAC CCA G | 199 |
| GHH LB 36 | GAT CAG CCT CGA CTG TGC CTT CTA GTT GCC A | 200 |
| GHH CP 1 | GAC TCA CAT TAG TCG GCC CGC | 201 |
| GHH CP 2 | CCT GGC TCA TTG ACC ACT TGG TAT GCC CCA GTA CA | 202 |
| GHH CP 3 | GAC CTT ATG AAC ACA GCA GGG ACT TTC CCC CGC ATT | 203 |
| | GAA CAT TGA CGT C | |
| GHH CP 4 | CTG GCA TCA GTA CAT CAA GTG CAA TAG GGA CTT TCG | 204 |
| | ACC GCC TGG AGT T | |
| GHH CP 5 | TAT TGA CGT CAA TGA CTG CCA AGT ACG CCC CGT ATG | 205 |
| | T | |
| GHH CP 6 | TCT ACG TGG GGA TTG TTT GTT TTG ACG CAA ATG GG | 206 |
| GHH CP 7 | AAT GGG ATC CAA GTC TCC ACC TAC TTG GAG TAC AT | 207 |
| GHH CP 8 | CCC CGC CGT AAC GCT ATC ATA GGT AAA TAT CGC TA | 208 |
| GHH CP 9 | CCG CGT TCA GAT ATA CGC GTT CGC TAA TAC GAC TC | 152 |
| GHH CP 10 | TCC GCC CCA TTG GCA CCA AAA TCG CGG TTT | 209 |
| GHH CP 11 | CAG TAC ATC AAT GGG CGG TGA TGC GGT TTT GG | 144 |
| GHH CP 12 | CTA GAG ATT GGT TTA GTG AAC CGT CAG ATC CG | 164 |
| GHH CP 13 | ACA TAA CTT ACG GTA AAT GGC CCG | 210 |
| GHH CP 14 | CCG CGG CGA CAT TGC CAT ATA CAA CGA C | 211 |
| GHH CP 15 | GTA AAC TGC CGT CAA TGG GTG GAG TAA TCA CTC ATC | 212 |
| | TTT ACG | |
| GHH CP 16 | ATT AAT AAC GGG GTG TCA ATA AT | 213 |
| GHH CP 17 | GGA GGT CTA TAT AAG CAG AGC | 159 |
| GHH CP 18 | TCC CAT ACA ATG ACC ATT AGT | 214 |
| GHH CP 19 | CGG TAG GCG CTG GCG TGC CTG TAC GGT GAC TAT AGG | 215 |
| | GAG AGC CGC CAC C | |
| GHH CP 20 | TTA CCA TGT GGA TAA ACG GGA TGT CGT AAC AAC | 216 |
| GHH CP 21 | TCA TAG CAT TAT TGA CTA GTT | 217 |

TABLE 10
Staple List for DNA Nanoparticle Construction

| DNA | |
|---|---|
| Nanoparticle | Staple List |
| GHL PO | GHL 2, 5, 6, 8, 11, 13, 17, 22, 24, 26, 27, 29, 32-35, 42 |
| GHL HS | GHL 1-3, 5, 6, 8, 10-14, 17, 19, 22-35, 38-44 |
| GHL FS | GHL 1-45 |
| GHL FSC | GHL 1, 2, 4-15, 17-20, 22, 24, 26-30, 32-39, 42, 44 |
| | GHL FSC 1-13 |
| GHH | GHH 1-23 |
| | GHL 1, 4, 7, 9, 10, 12, 14, 15, 18-20, 28, 30, 36-39, 44 |
| | GHL FSC 3-5, 7, 8, 10, 12 |
| GHH LB | GHH 1-23 |
| | GHH LB 1-36 |
| GHH CP | GHL 1, 4, 7, 9, 10, 12, 14, 15, 18-20, 28, 30, 36-39, 44 |
| | GHL FSC 3-5, 7, 8, 10, 12 |
| | GHH CP 1-21 |

### DNA Nanoparticle Transfection

The transfection of DNA nanoparticles was performed in triplicate using HTR-8/SVneo (ATCC CRL-3271) cells and jetPRIME® transfection reagent (VWR, 89129-924). Briefly, 50,000 cells were seeded per 24-well plate and transfections were performed after 24 hours using 141.5 fmol of origami samples with a 1:2 ratio of the jetPRIME® reagent. Media was replaced 24 hours after transfection and images were collected 48 hours after transfection (Olympus Fluorescent Microscope, AST-0008943). Raw images were analyzed using Fiji software (Schindelin et al., “Fiji: An Open-Source Platform for Biological-Image Analysis,” Nat. Methods 9: 676-682 (2012), which is hereby incorporated by reference in its entirety). For each sample image, raw images of fluorescent and non-fluorescent cells were captured and overlayed using Fiji's Merge Channel tool. The background noise from each merged image was removed using the brightness and contrast tool. The raw images of non-fluorescent cells were used to count the total number of cells per image, and the merged images were used to count GFP-positive cells per image. ImageJ was used for counting. The number of cells, both fluorescent and non-fluorescent, were counted manually, and the efficiency of gene expression was calculated by dividing the number of GFP-positive cells by the total number of cells for each image.

Flow cytometry. Data acquisition and analysis were conducted using an unmodified BD FACSCanto flow cytometer (BD Biosciences, San Jose, CA) with a 488 nm laser for GFP excitation and measurement of cell forward (FSC) and side scatter (SSC) properties. Green fluorescence was collected through a 525/50 nm band pass (BP) filter, while forward and side scatter data was acquired using 488/10 nm BP filters. Voltages for scatter and fluorescence detectors was adjusted based on untreated or mock-transfected cells. Data from Negative controls was used to differentiate GFP(−) and GFP(+) events. GFP fluorescence data was presented on four-decade log scales with bi-exponential scaling, while FSC and SSC was displayed using linear amplification. To differentiate intact cells from debris and other extraneous events, FSC and SSC properties of cells was used to establish analysis standards. Data acquisition employed a medium flow rate of 60 μl/minute, collecting a minimum of 10,000 gated “cell” events for each sample. FACSDiva software (ver. 8.0.1, BD Biosciences) was used for data analysis, and percentages of gene expression were adjusted relative to the expression of positive control.

### Example 5-DNA Nanoparticle Construction, Purification, and Verification

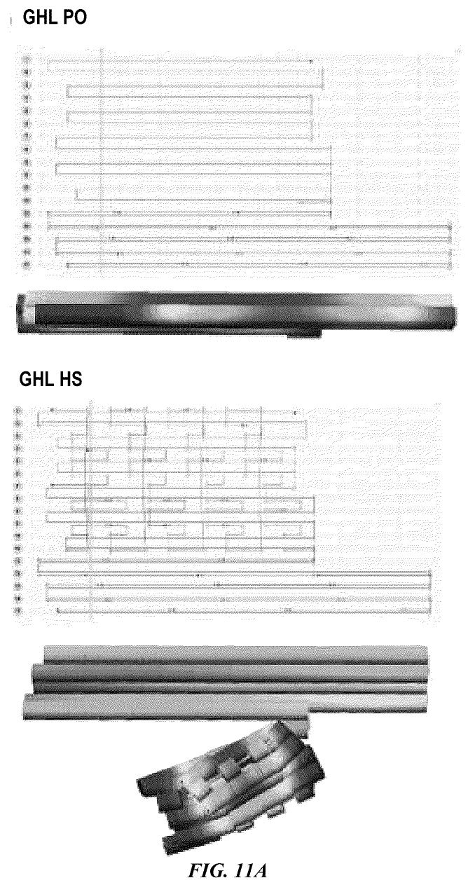

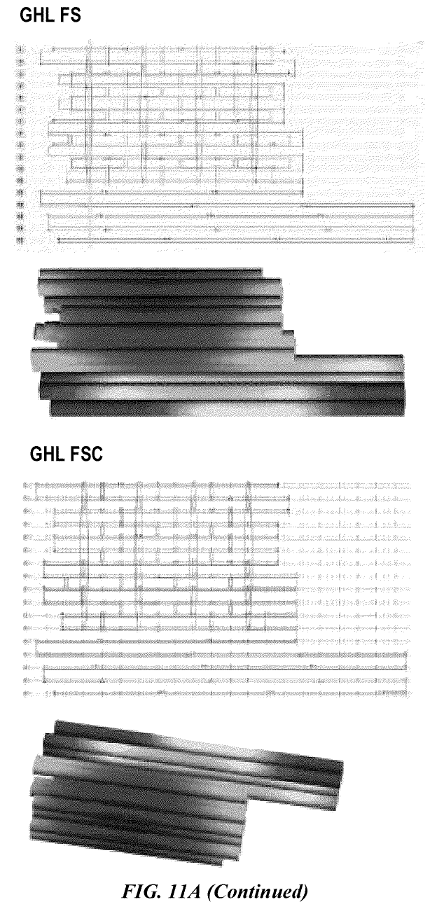

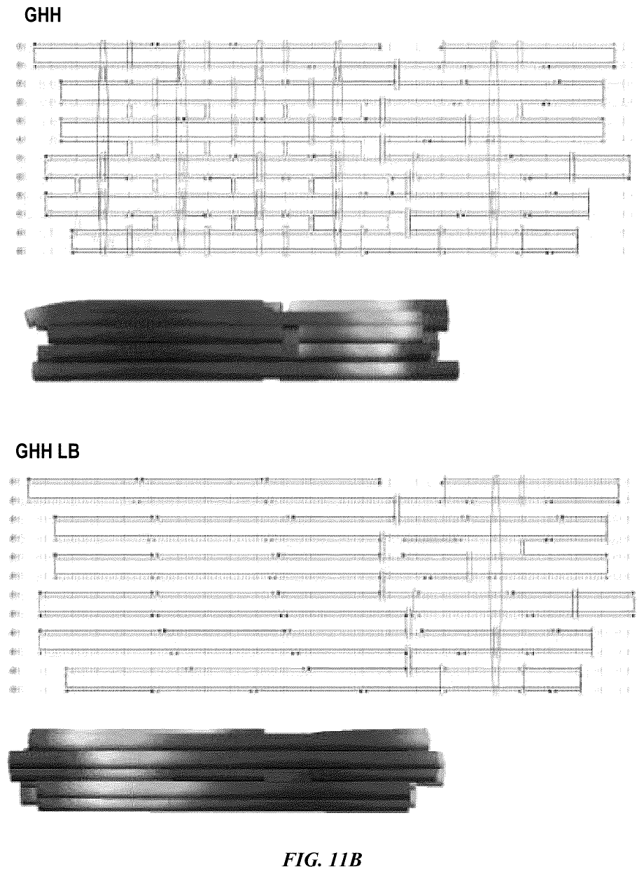

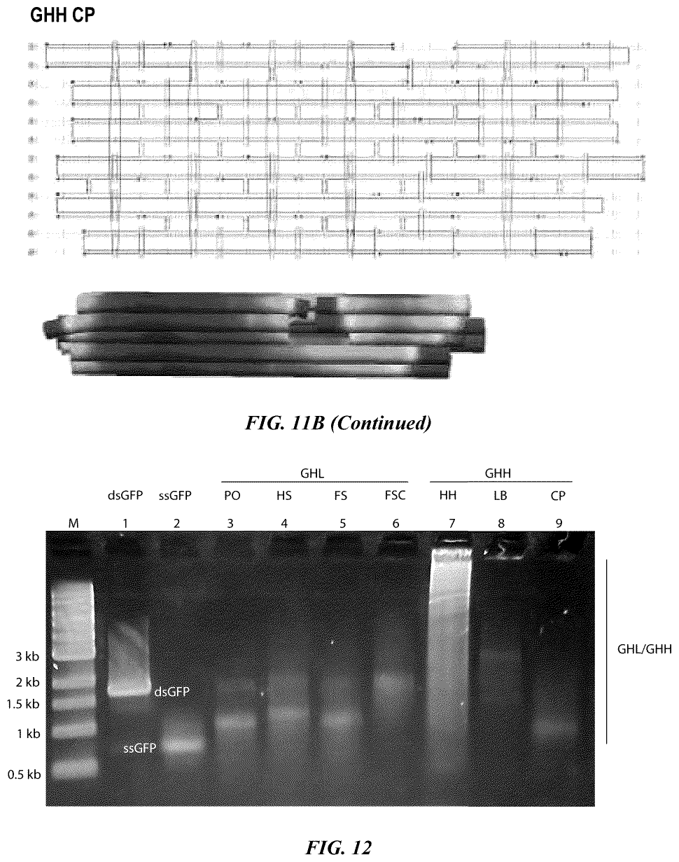

To assess the capability of DNA nanoparticles to be able to express embedded genes in cell culture, a custom scaffold was synthesized by asymmetric PCR (aPCR) as previously described (Oh and Henderson, “A Comparison of Methods for the Production of Kilobase-Length Single-Stranded DNA,” DNA 2:56-67 (2022), which is hereby incorporated by reference in its entirety). The scaffold contained a Cauliflower Mosaic Virus (CMV) promoter and a CMV enhancer to support gene expression in human cells, and the Eukaryotic Green Fluorescent Protein (EGFP) gene for fluorescence-based visualization of gene expression. The design of DNA nanoparticles in this study was carried out using caDNAno (Douglas et. al., “Rapid Prototyping of 3D DNA-Origami Shapes with caDNAno,” Nucleic Acids Res. 37:5001-5006 (2009), which is hereby incorporated by reference in its entirety). Two sets of DNA nanoparticles were created to examine the individual contributions of gene components (i.e., promoter/enhancer and gene) toward gene expression. The overall architecture was roughly cylindrical, featuring a consistent crossover pattern across various forms of the structure (FIGS. 11A-11B). The first set, referred to as GHL (GFP gene Honeycomb structure with a Linear promoter and enhancer), incorporated linear duplex promoter and enhancer domains (i.e., not folded using staple oligonucleotides) to specifically investigate the impact of a folded architecture on the elongation phase of transcription. A positive correlation between the abundance of ssDNA domains within DNA nanoparticles and overall levels of transcription in a nuclease-free in vitro system was previously observed (Oh and Henderson, “In Vitro Transcription of Self-Assembling DNA Nanoparticles,” Sci. Rep. 13(1): 12961 (2023), which is hereby incorporated by reference in its entirety). It was hypothesized that the increased structural flexibility conferred by ssDNA in that system facilitated polymerase activity. However, ssDNA is generally more susceptible to nuclease-mediated scission than dsDNA (Chandrasckaran A R., “Nuclease Resistance of DNA Nanostructures,” Nat. Rev. Chem. 5:225-239 (2021) and Anindya R., “Single-Stranded DNA Damage: Protecting the Single-Stranded DNA from Chemical Attack,” DNA Repair (Amst) 87:102804 (2020), which are hereby incorporated by reference in their entirety). Therefore, to assess the influence of ssDNA domains on gene expression in a cellular milieu, multiple DNA nanoparticles with an identical overall design but with varying levels of ssDNA were prepared. These variants were labeled as GHL PO (DNA nanoparticle with linear duplex promoter/enhancer with single-stranded gene; Promoter/enhancer domains were Only duplex within the structure), GHL HS (DNA nanoparticle with linear duplex promoter/enhancer and a half complement of staples; Half-Set of staples used), and GHL FS (DNA nanoparticle with linear duplex promoter/enhancer and a mostly full complement of staples; Full-Set of staples used), in order of increasing number of staples. Notably, although GHL FS had the greatest number of staples among the aforementioned DNA nanoparticles, it retained ssDNA domains at the ends of duplex DNA helices to enhance flexibility and potentially facilitate folding (Yang et al., “A Single Strand: A Simplified Approach to DNA Origami,” Front. Chem. 11:1126177 (2023), which is hereby incorporated by reference in its entirety). Finally, to fully eliminate ssDNA domains within the nanoparticle, the staples at the edges of GHL FS were replaced for the full coverage of ssDNA domains, resulting in the GHL FSC (DNA nanoparticle with linear duplex promoter/enhancer, a full complement of staples (Full-Set of staples used), and fully Covered single-stranded end of helices) variant.

The second set, designated as GHH (GFP gene Honeycomb structure with Honeycomb-loop folded promoter and enhancer), included the promoter and enhancer domains within the folded architecture of the DNA nanoparticles. This set contained DNA nanoparticles with linear and folded genes to evaluate contributions from the initiation phase (folded promoter/enhancer with linear duplex gene) and from both initiation and elongation phases together (all components included in the folded architecture). The structure with a folded promoter/enhancer and linear gene was denoted as GHH LB (DNA nanoparticle with a loosely folded promoter/enhancer and a Linear duplex gene Body). Two folding approaches were employed to evaluate the importance of transcription factor binding sites: GHH and GHH CP. In GHH, the promoter and enhancer were folded to match the cylindrical shape of the folded gene, but avoiding crossovers at the transcription factor binding sites. This allowed unimpeded binding of transcription factors and overall flexibility at the promoter and enhancer domains presumably providing sufficient conformational flexibility of the structure to facilitate the formation of the transcription initiation complex. Conversely, GHH CP (DNA nanoparticle with Compactly folded Promoter/enhancer and gene) was designed with a maximum number of crossovers in this region to test whether this creates a steric impediment sufficient to compromise transcription factor binding. To avoid the impact of ssDNA domains, all GHH variants were entirely double-stranded. All of the structures and their acronyms are listed in Table 11 and displayed in FIGS. 11A-11B.

TABLE 11
Names of DNA Samples Used in Examples 5 and 6

| Name | Contents |
|---|---|
| dsGFP | Fully linear double-stranded GFP gene. |
| dsGFP-a | Fully linear double-stranded GFP gene, produced as a byproduct of aPCR |
| ssGFP | Single-stranded antisense strand for nanoparticle scaffold. |
| GHL PO | DNA nanoparticle with a linear duplex promoter/enhancer and single-stranded |
| | GFP gene; variant of GFP gene Honeycomb structure with a Linear promoter |
| | and enhancer, Promoter/enhancer domains are Only duplexes within the |
| | structure. |
| GHL HS | DNA nanoparticle with a linear duplex promoter/enhancer and a half |
| | complement of staples (i.e., loosely folded); variant of GFP gene Honeycomb |
| | structure with a Linear promoter and enhancer, the structure folded with a Half- |
| | Set of staples. |
| GHL FS | DNA nanoparticle with a linear duplex promoter/enhancer and a mostly full |
| | complement of staples (i.e., tightly folded with single-stranded edges); variant of |
| | GFP gene Honeycomb structure with a Linear promoter and enhancer, the |
| | structure folded with a Full-Set of staples. |
| GHL FSC | DNA nanoparticle with a linear duplex promoter/enhancer and a full complement |
| | of staples (i.e., tightly folded with no ssDNA domains); variant of GFP gene |
| | Honeycomb structure with a Linear promoter and enhancer, the structure folded |
| | with a Full-Set of staples and Covered single-stranded ends. |
| GHH | DNA nanoparticle with a loosely folded promoter/enhancer, tightly folded gene, |
| | and no ssDNA domains; GFP gene Honeycomb structure with a Honeycomb- |
| | loop folded promoter and enhancer. |
| GHH LB | DNA nanoparticle with a loosely folded promoter/enhancer, linear duplex gene, |
| | and no ssDNA domains; variant of GFP gene Honeycomb structure with a |
| | Honeycomb-loop folded promoter and enhancer, the structure with a Linear |
| | Body gene. |
| GHH CP | DNA nanoparticle with a tightly folded promoter/enhancer and gene, and no |
| | ssDNA domains; variant of GFP gene Honeycomb structure with a Honeycomb- |
| | loop folded promoter and enhancer, the structure with Compactly folded |
| | Promoter and enhancer. |

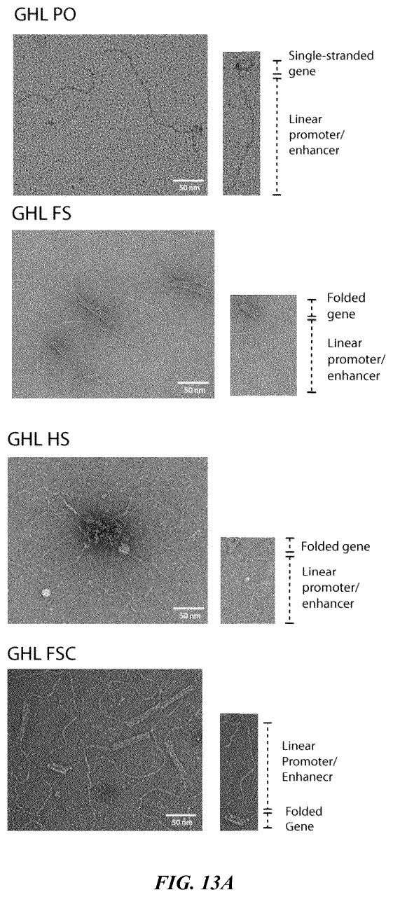

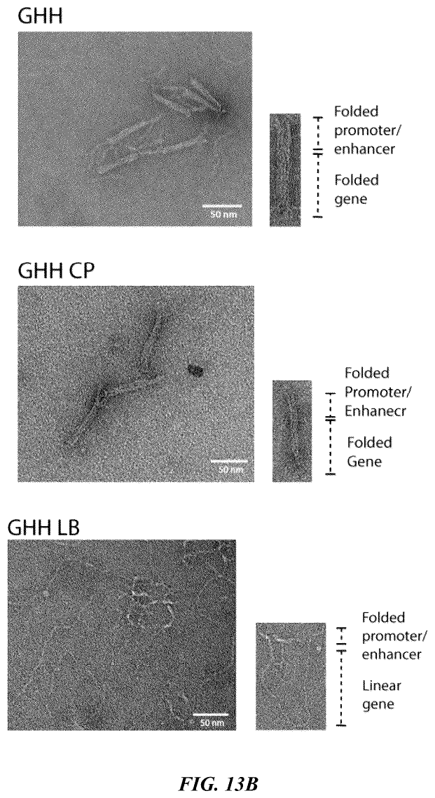

To mitigate any influence of the presence of excess staples, they were removed by ultrafiltration and/or differential PEG precipitation. Electrophoretic analysis confirmed effective staple elimination. However, some nanoparticle samples showed noticeable smearing, which often indicates sample aggregation. Notably, even considering aggregation, GHH exhibited the most extreme smearing, which raised concerns regarding the structural integrity of this sample (FIG. 12). Nevertheless, TEM imaging verified that the DNA nanoparticle samples remained structurally intact and verified the occurrence of sample aggregation which correlated with the observed smearing. This aggregation was likely attributable to end-to-end adhesion (pi bond-stacking) in these samples, a phenomenon known to occur in DNA origami with blunt-ended helices (Baptist and Heuer-Jungemann, “Lyophilization Reduces Aggregation of Three-Dimensional DNA Origami at High Concentrations,” ACS Omega 8:18225-18233 (2023), which is hereby incorporated by reference in its entirety; FIGS. 13A-13B). In addition, the high flexibility observed at one end of GHH may be attributed to the minimal number of crossovers at the promoter/enhancer regions, potentially contributing further to the smearing pattern observed during electrophoresis (FIGS. 13A-13B) (Yang et al., “A Single Strand: A Simplified Approach to DNA Origami,” Front. Chem. 11 (2023) and Baptist and Heuer-Jungemann, “Lyophilization Reduces Aggregation of Three-Dimensional DNA Origami at High Concentrations,” ACS Omega 8:18225-18233 (2023), which are hereby incorporated by reference in their entirety).

### Example 6-Gene Expression of DNA Nanoparticles in Cultured Cells

Gene expression of DNA nanoparticles was evaluated by transfection of a human trophoblast cell line in culture and subsequent image analysis of GFP production. Two independent transfections were made using DNA nanoparticles samples in either TAEM buffer or PBSM buffer. For DNA nanoparticles suspended in TAEM buffer, transfection was done with single biological replicate and three technical replicates. After confirming consistency among technical replicates, three representative images were taken from one of the replicates.

Gene expression was quantified by counting the number of GFP-positive cells, irrespective of fluorescence intensity. As a positive control, a linear duplex GFP PCR product (dsGFP) was used to establish the maximum gene expression potential for this gene ensemble under the transfection conditions used. A single-stranded antisense strand (ssGFP) comprised of the bare origami scaffold was employed as a negative control based on the observation that ssDNA is highly susceptible to nuclease degradation in cell culture and should, therefore, manifest the minimal level of gene expression, if any (Chandrasekaran A R, “Nuclease resistance of DNA nanostructures.” Nat Rev Chem 5:225-239 (2021) and Anindya R, “Single-stranded DNA damage: Protecting the single-stranded DNA from chemical attack,” DNA Repair (Amst) 87: 102804 (2020), which are hereby incorporated by reference in their entirety).

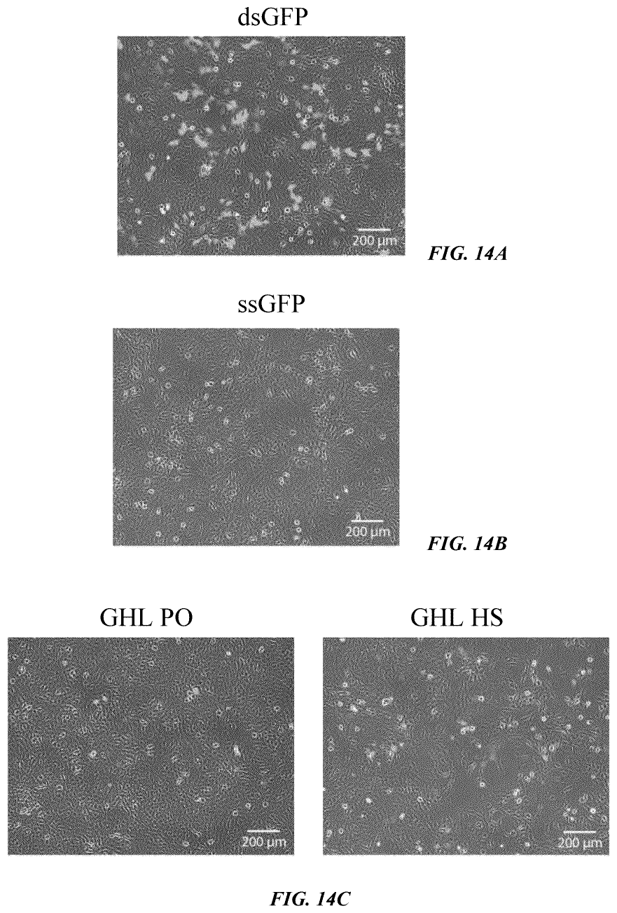

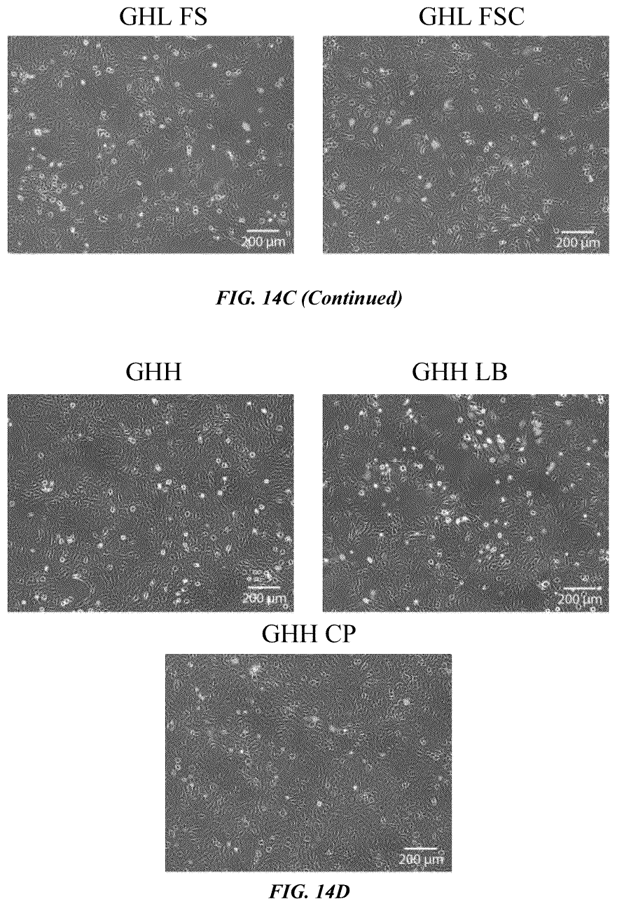

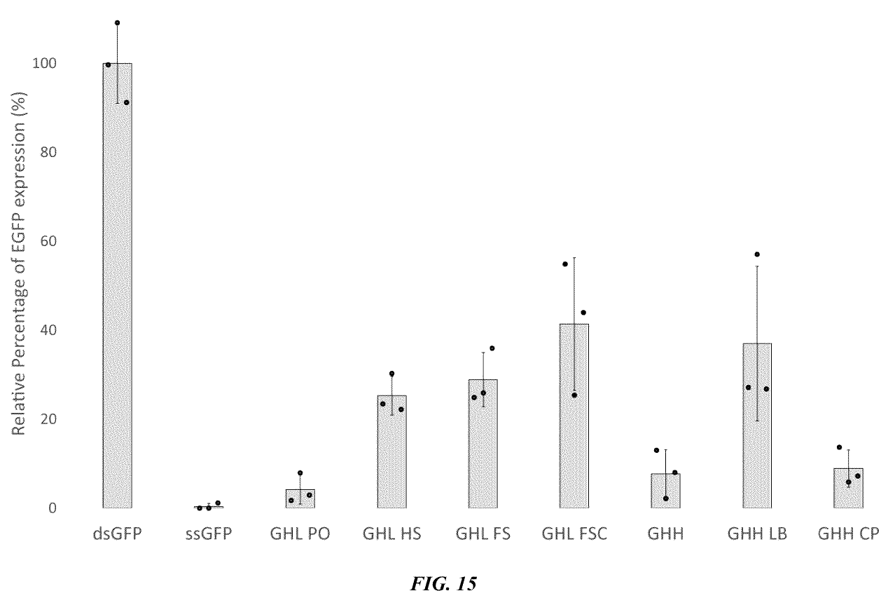

As expected, the ssGFP wells exhibited minimal GFP expression, with only one representative image showing three GFP-positive cells and no expression in the other two images. Comparing DNA nanoparticles with linear duplex promoters and enhancers (GHL), increased coverage of ssDNA domains and “compactness” positively correlated with enhanced gene expression (FIGS. 14A-14C). This trend was quantitatively assessed by counting cells (an average of 2265 cells per image; FIG. 15). Notably, there was only a slight increase in gene expression of GHL FS relative to GHL HS despite a substantial difference in staple coverage and, presumably, overall nanoparticle compaction. In contrast, complete coverage of ssDNA domains (GHL FSC) led to a significant enhancement in gene expression, underscoring the negative impact of even a small number of ssDNA domains, at least when exposed at the ends of duplexes.

Gene expression was observed in all cases in which DNA nanoparticles contained folded promoters and enhancers. Surprisingly, the difference in terms of gene expression between GHH and GHH CP (±staple crossovers in the promoter region, respectively) was subtle. Interestingly, GHH CP exhibited slightly increased gene expression despite the presence of DNA crossovers at transcription factor binding sites, which might naively be assumed to create a steric impediment to transcription initiation (FIG. 14D).

For transfection of DNA nanoparticles suspended in PSBM buffer, each sample was transfected in three to four biological replicates and two technical replicates. For analysis of gene expression, flow cytometry was used to quantify the number of GFP-positive cells in addition to the evaluation of average GFP brightness per sample and cell size distribution. As a positive control, a linear duplex GFP PCR product (dsGFP) was used to establish the maximum gene expression potential for this gene ensemble under the transfection conditions used. Since the generation of an origami scaffold by aPCR involved different polymerases, a linear duplex GFP aPCR byproduct (dsGFP-a) was also evaluated for a more accurate comparison of gene expression efficiency between DNA samples. Although partly serving as a positive control, dsGFP-a exhibited a reduced level of gene expression compared to another positive control, dsGFP. This possibly implies that the use of a lower fidelity polymerase resulted in gene products that were less efficient in terms of gene expression, presumably due to introduction of sequence errors.

A single-stranded antisense strand (ssGFP) comprised of the bare origami scaffold was employed as a negative control based on the previous reports that ssDNA is highly susceptible to nuclease degradation in cell culture and should, therefore, result in the minimum level of gene expression, if any (Chandrasekaran, AR., “Nuclease Resistance of DNA Nanostructures,” Nat. Rev. Chem. 5:225-239 (2021) and Anindya R., “Single-Stranded DNA Damage: Protecting the Single-Stranded DNA from Chemical Attack,” DNA Repair (Amst) 87:102804 (2020), which is hereby incorporated by reference in its entirety). Although the ssGFP exhibited some level of gene expression as expected, it was substantially lower than the experimental samples. In general, DNA nanoparticles supported gene expression, with efficacy correlating with robustness observed in previous reports on the expression of DNA nanoparticles in mammalian cells.

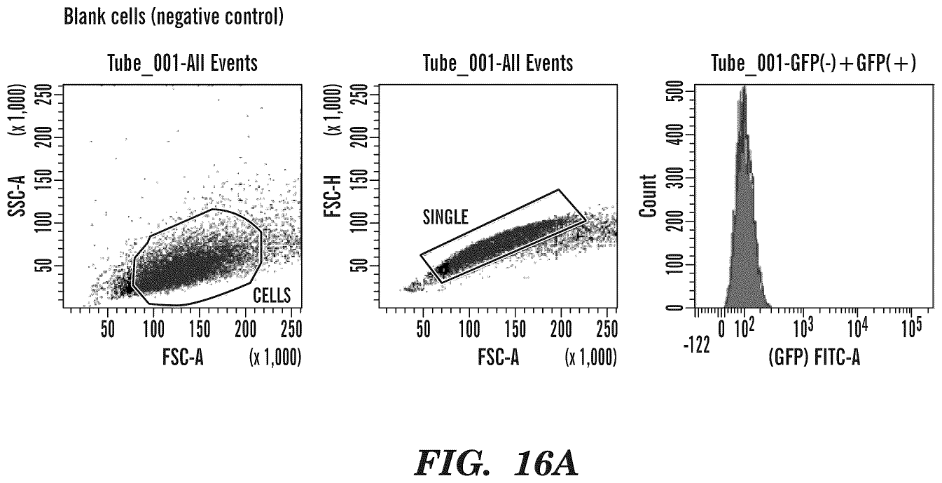

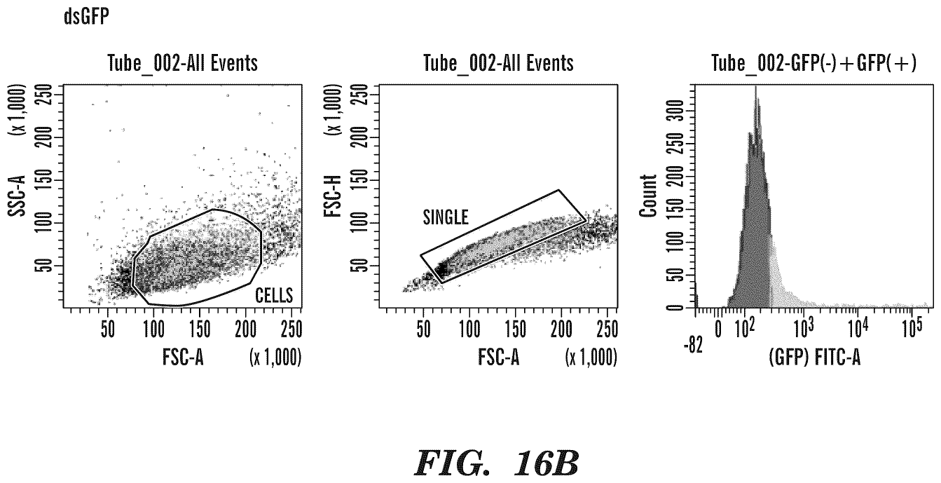

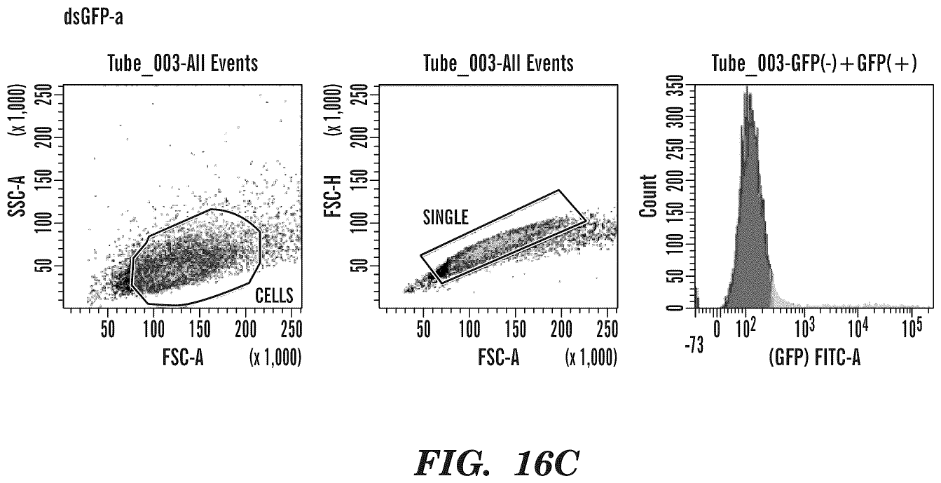

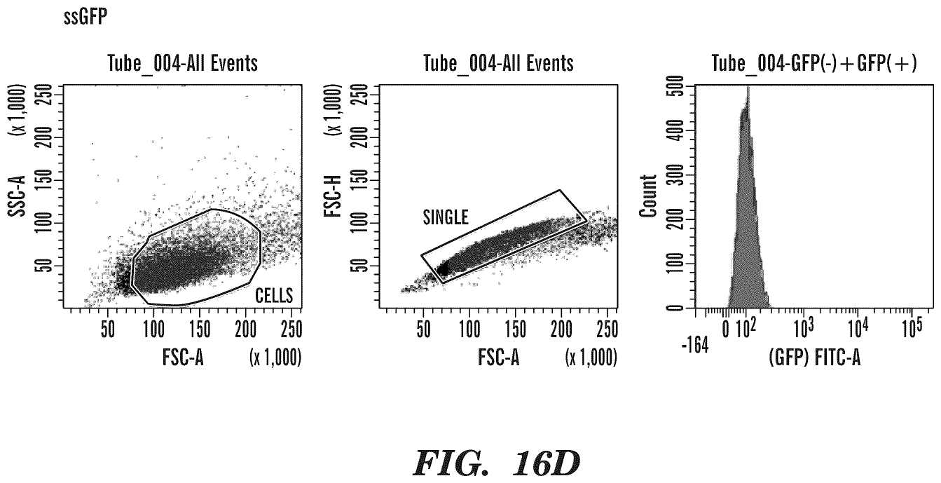

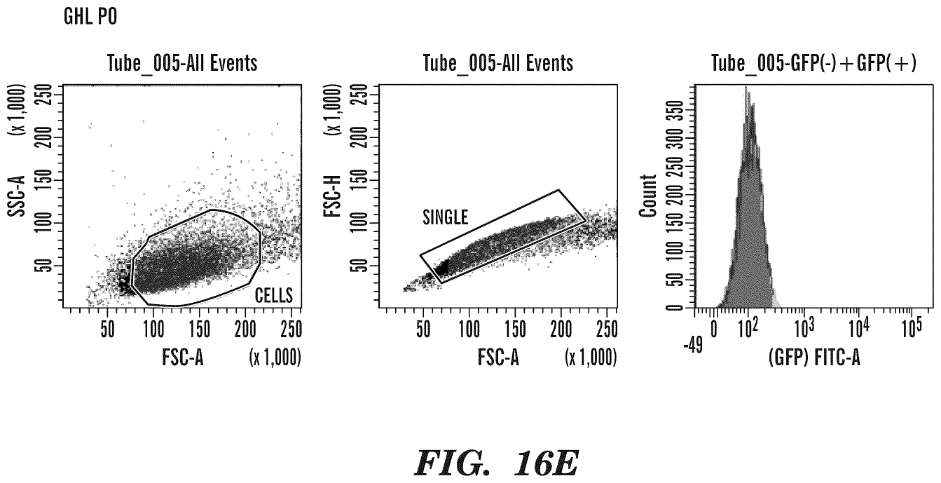

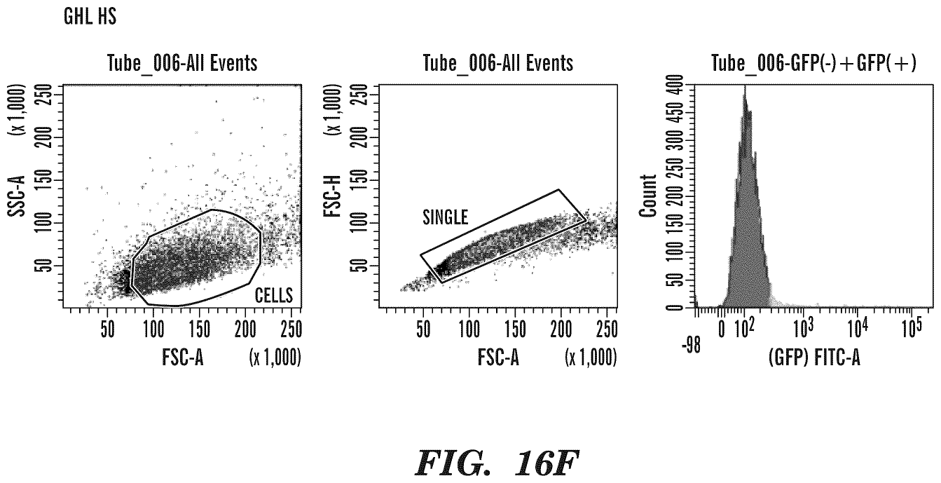

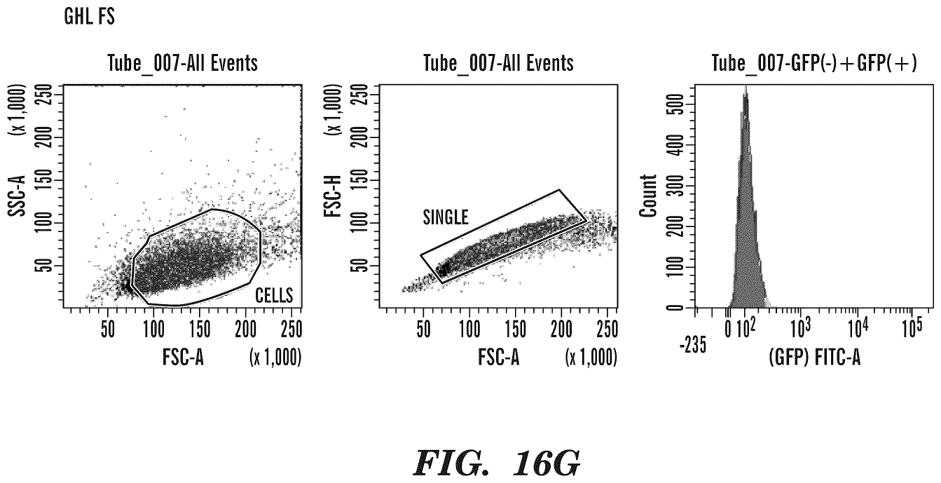

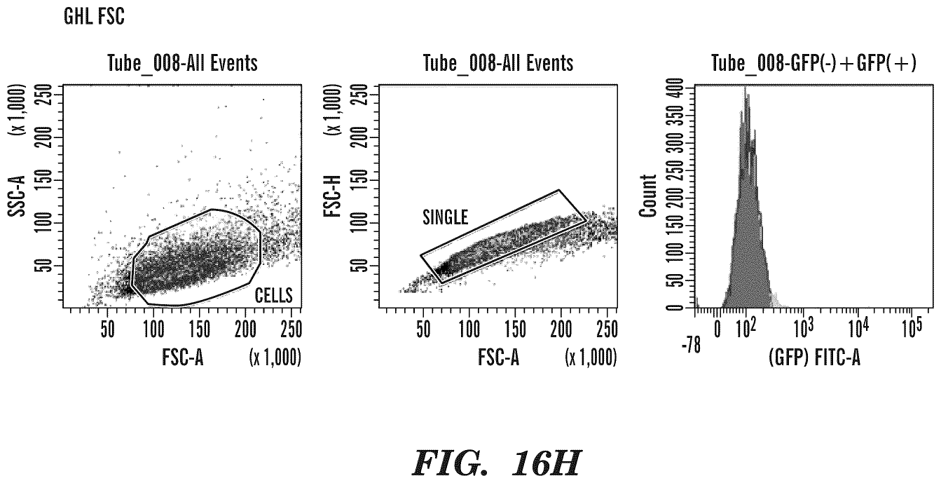

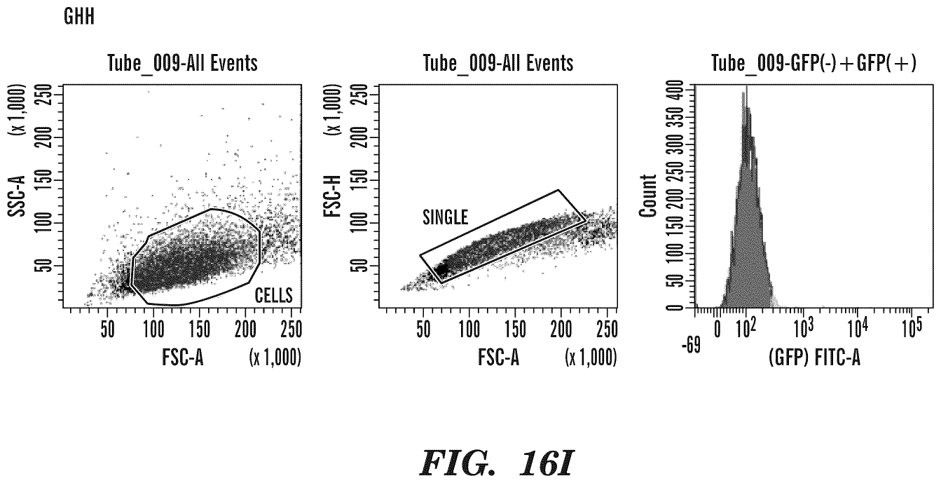

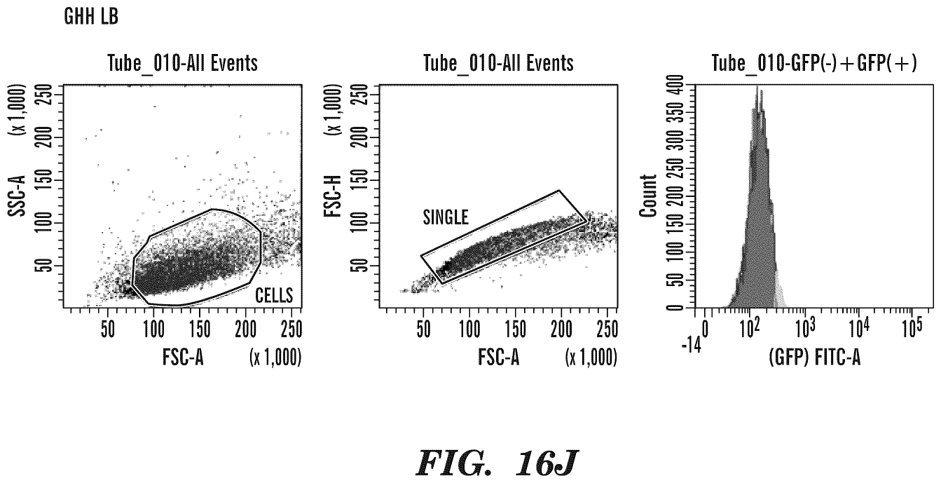

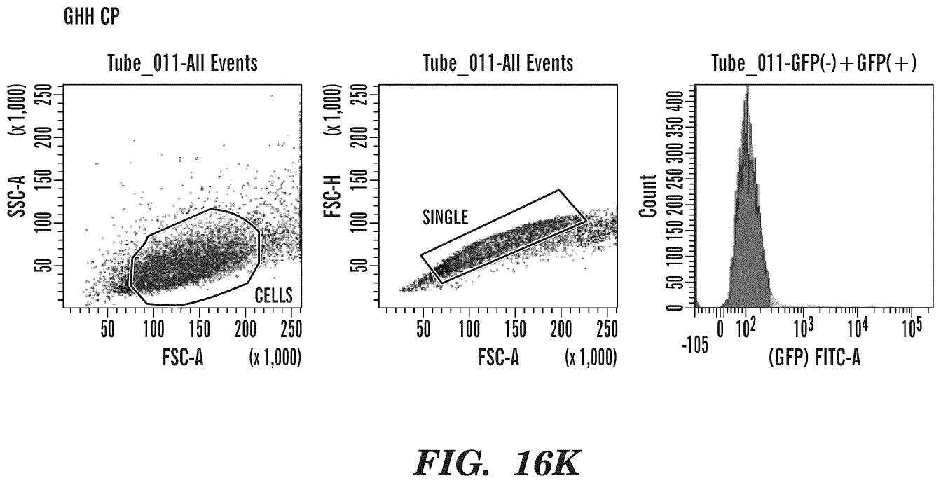

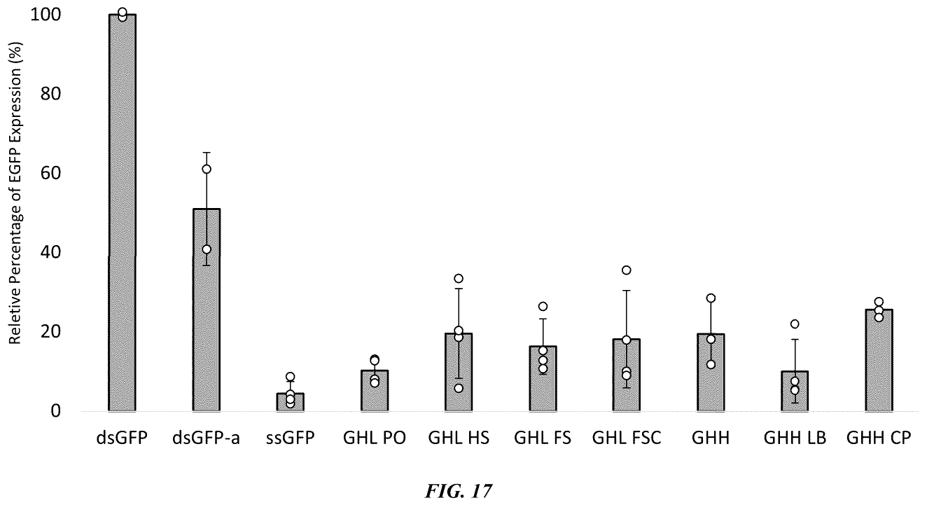

It was further observed that nanoparticles architectures with a line gene domain, regardless of whether they were single- or double-stranded, exhibited comparatively lower levels of gene expression (FIGS. 16A-16K). This trend was quantitatively assessed by flow cytometry analysis (FIG. 17). Flow cytometry further revealed that GFP-expressing cells transfected with DNA nanoparticles exhibited generally less bright fluorescent signals than positive controls. This possibly correlates with lesser efficacy in terms of gene expression from DNA nanoparticles. In addition to fluorescence, flow cytometry indicate a relatively constant cell size distribution among DNA samples. Thus, there was no apparent phenotypic changes to the cells upon transfection by DNA nanoparticles.

### Discussion of Examples 5 and 6

Self-assembling DNA nanoparticles represent a potentially powerful platform for exploring the uses of architecturally engineered genetic material in living systems (Kretzmann et al., “Gene-Encoding DNA Origami for Mammalian Cell Expression,” Nat. Commun. 14:1017 (2023); Lin-Shiao et al., “CRISPR-Cas9-Mediated Nuclear Transport and Genomic Integration of Nanostructured Genes in Human Primary Cells,” Nucleic Acids Res. 50:1256-1268 (2022); Wu et al., “Genetically Encoded DNA Origami for Gene Therapy In Vivo,” J. Am. Chem. Soc. 145:9343-9353 (2023); and Liedl et al., “Active Nuclear Import of Mammalian Cell-Expressible DNA Origami,” J. Am. Chem. Soc. 145:4946-4950 (2023), which are hereby incorporated by reference in their entirety). Examples 5 and 6 explored the impact of various DNA nanoparticle design parameters on gene expression in cell culture. These parameters include the presence of ssDNA domains, and the impact of folding the promoter/enhancer and coding region of a gene, independently and in combination. The results of Examples 5 revealed that the presence of ssDNA domains in DNA nanoparticles has an adverse impact on gene expression. Theoretically, a single cleavage by an endonuclease can significantly compromise gene expression. Thus, an increased number of exposed ssDNA domains increases the likelihood of nuclease degradation, ultimately leading to reduced gene expression, as observed in FIGS. 14B-14C and FIG. 15. In addition to the elevation of gene expression upon staple binding (i.e., formation of duplex domains), the compact folding of DNA further enhances gene expression, presumably due to enhanced resistance to nucleases (Chandrasekaran, AR., “Nuclease Resistance of DNA Nanostructures,” Nat. Rev. Chem. 5:225-239 (2021), which is hereby incorporated by reference in its entirety).

Surprisingly, despite GHL FS having approximately twice the staple coverage of GHL HS, the overall level of gene expression observed for these two constructs was comparable. The locations of ssDNA domains (i.e., relative accessibility to nuclease attack) may be relevant in this context. ssDNA domains exposed at the end of helices may be more accessible than those at internal positions. Thus, the similar levels of gene expression of GHL FS and GHL HS may be the consequence of comparable terminal ssDNA domains (FIG. 11A) with internal ssDNA domains playing a secondary role that is obscured by rapid digestion of terminal ssDNA domains.

Furthermore, Given the versatile capacity of cells observed in previous study (Kretzmann et al., “Genc-Encoding DNA Origami for Mammalian Cell Expression,” Nat. Commun. 14:1017 (2023), which is hereby incorporated by reference in its entirety), it is reasonable to expect gene expression from a nanoparticle with tightly folded promoter and enhancer (GHH CP), which was designed to significantly attenuate, but not preclude transcription factor binding. Surprisingly, GHH CP exhibited a more efficient gene expression than GHH, a comparable nanoparticle with more loosely folded promoter and enhancer domains, although the effect was subtle. Compact DNA nanoparticles, like those tested in this study, are known for their increased resistance to nuclease activity (Chandrasekaran, AR., “Nuclease Resistance of DNA Nanostructures,” Nat. Rev. Chem. 5:225-239 (2021), which is hereby incorporated by reference in its entirety). While both GHH and GHH CP lack ssDNA domains, the minimal number of crossovers in GHH is consistent with a less rigid architecture (i.e., flexible helices) at the promoter/enhancer domain (Xin et al, “Environment-Dependent Stability and Mechanical Properties of DNA Origami Six-Helix Bundles with Different Crossover Spacings,” Small 18:2107393 (2022), which is hereby incorporated by reference in its entirety) as observed in TEM (FIG. 13B). This could cause a slightly increased susceptibility to nuclease degradation. This susceptibility could impair gene function and lead to the slight reduction in gene expression that was observed (FIG. 14D and FIG. 15).

DNA nanoparticles demonstrated substantial gene expression when the gene and promoter/enhancer were folded independently. However, the collective folding of the gene and promoter/enhancer resulted in a notable decrease in overall gene expression. This suggests an additive effect of folding each subunit (promotor/enhancer and coding portion of the gene) collectively contributed to a reduction in gene expression. One plausible explanation for this observation is that the tightly constrained configuration between folded promoter/enhancer and gene caused by the staple design may have created unfavorable conditions for RNA polymerase during the transition from transcription initiation to elongation. This observation implies that gene expression efficiency may be regulated by nuancing the connectivity between the promoter/enhancer region and the coding portion of the gene. These findings establish a foundation for designing DNA nanoparticle architectures to modulate gene expression and suggest that these design features may be further fine-tuned.

However, when DNA nanoparticles buffer was exchanged to 1×PBSM buffer, it resulted in different expression pattern. Comparison between GHL PO and other DNA nanoparticle samples revealed that once the staple coverage exceeds a certain threshold, the remaining fraction of single-stranded domains did not appear to cause any obvious adverse impact on gene expression (FIGS. 16A-16H and FIG. 17). One plausible explanation for this phenomenon is the potentially enhanced resistance to nuclease attack upon incubation of DNA nanoparticle samples under 1×PBS buffer as observed in previous reports (Linko et al, “Stability of DNA origami Nanostructures in Physiological Media: The Role of Molecular Interactions,” Small 19(34): 2301935 (2023), which is hereby incorporated by reference in its entirety). This was partly supported as the previous experiments on transfection DNA nanoparticles that were incubated in 1×TAEM buffer resulted in a more obvious trend of increasing trend of gene expression upon more duplex coverage of single-stranded domains.

It is generally believed that DNA crossovers create steric hindrances that could potentially impede the progression of polymerases. In a sense, linear duplex DNA (i.e., no crossovers) showed a maximum degree of gene expression, and a previous study showed the highest level of transcription with loose architectures. However, surprisingly, GHL PO and GHH LB, the DNA nanoparticles with minimum crossovers, showed comparatively lower levels of gene expression. As pointed out above, the majority of GHL PO structure was comprised of single-stranded domains making it more vulnerable to nuclease degradation, potentially resulting in lower level of gene expression. GHH LB, on the other hand, was not only fully comprised of double-stranded domains, but also had the most loosened architecture. Nonetheless, counterintuitively, GHH LB, on average, exhibited the lowest level of gene expression among all DNA nanoparticle samples. It is plausible that various nicks introduced to the structure by binding of discontinuous staples rendered the architecture to be slightly more susceptible to nucleases. While folding DNA into compact architecture reduces its susceptibility to nucleases, as GHH LB remained as lincar nicked duplex, it is possible that it retained the same degree of susceptibility to nucleases, potentially resulting in decreased level of gene expression. This could be partly supported by how GHH CP, the most compact architecture, showed the highest level of gene expression.

A recent study by Kretzmann et al. demonstrated the remarkable ability of cells to process complex structured genes. Particularly striking was the observation that DNA nanoparticles constructed using sense strand scaffolds, rather than the gene-encoding antisense strand, still expressed the encoded gene within cells. This would require that the sense strand is first copied to create the template strand which may then be transcribed to create the gene product thus suggesting that DNA nanoparticles are both transcription- and replication-competent in cell culture (Kretzmann et al., “Gene-Encoding DNA Origami for Mammalian Cell Expression,” Nat. Commun. 14:1017 (2023), which is hereby incorporated by reference in its entirety). It is noteworthy that in that case, the constructs employed included a eukaryotic origin of replication whereas the constructs used in the present study did not. Thus, the latter may be limited to transcription competence but not capable of replication. Adding on to the versatile capacity of cells observed in the study, various architectures used in this study in which two major components involved in transcription, ultimately gene expression (promoter and enhancer, and gene) folded both independently and altogether exhibited relatively similar efficiencies in terms of gene expression while opening the possibility of adjusting the flexibility for tuning the architecture to be flexible enough to allow efficient gene expression while making them rigid enough to prevent any nuclease degradation.

It is noteworthy that the chemical malleability of DNA nanoparticles enables the incorporation of diverse molecular and chemical species, offering advantages such as enhanced cellular uptake, improved stability, and intra-/intermolecular targeting (Mathur et al., “Uptake and Stability of DNA Nanostructures in Cells: A Cross-Sectional Overview of the Current State of the Art,” Nanoscale 15:2516-2528 (2023); Stephanopoulos N., “Strategies for Stabilizing DNA Nanostructures to Biological Conditions,” ChemBioChem 20:2191-2197 (2019); Mikkilä et al., “Virus-Encapsulated DNA Origami Nanostructures for Cellular Delivery,” Nano Lett. 14:2196-2200 (2014); Jiang et al., “Rationally Designed DNA-Origami Nanomaterials for Drug Delivery In Vivo,” Adv. Mater. 31(45):c1804785 (2019); Xu et al., “Cellular Ingestible DNA Nanostructures for Biomedical Applications,” Adv. Nanobiomed. Res. 3:2200119 (2023); Udomprasert and Kangsamaksin, “DNA Origami Applications in Cancer Therapy,” Cancer Sci. 108:1535-1543 (2017); and Lucas et al., “DNA Origami Nanostructures Elicit Dose-Dependent Immunogenicity and Are Nontoxic up to High Doses In Vivo,” Small 18 (2022), which are hereby incorporated by reference in their entirety). For example, coating DNA nanoparticles with lipid moieties through electrostatic interactions enhances stability under physiological conditions and promotes increased cellular uptake (Wu et al., “Genetically Encoded DNA Origami for Gene Therapy In Vivo,” J. Am. Chem. Soc. 145:9343-9353 (2023); Agarwal et al., “Block Copolymer Micellization as Protection Strategy for DNA Origami,” Angew. Chem. Int. Ed. Engl. 56(20):5460-5464 (2017); Wang et al., “DNA Origami Protection and Molecular Interfacing through Engineered Sequence-Defined Peptoids,” PNAS 117:6339-6348 (2020); and Bastings et al., “Modulation of the Cellular Uptake of DNA Origami through Control over Mass and Shape,” Nano Lett. 18(6):3557-3564 (2018), which are hereby incorporated by reference in their entirety). Wu et al. demonstrated this approach by coating DNA nanoparticles with lipid fatty acids to enhance cell penetration (Wu et al., “Genetically Encoded DNA Origami for Gene Therapy In Vivo,” J. Am. Chem. Soc. 145:9343-9353 (2023), which is hereby incorporated by reference in their entirety). Additionally, introducing nuclear localization signals (NLS) through intercalation or conjugation significantly enhances the nuclear import of transfected DNA, resulting in improved overall gene expression (Branden et al., “A Peptide Nucleic Acid-Nuclear Localization Signal Fusion that Mediates Nuclear Transport of DNA,” Nat. Biotechnol. 17:784-787 (1999); Zhang et al., “Intercalating Conjugates of PEG with Nuclear Localization Signal (NLS),” Polymer Prepr. 49(2):434-435 (2008); Zanta et al., “Genc Delivery: A Single Nuclear Localization Signal Peptide is Sufficient to Carry DNA to the Cell Nucleus,” PNAS 96(1):91-96 (1999); and Bogacheva et al., “Arginine-Rich Cross-Linking Peptides with Different SV40 Nuclear Localization Signal Content as Vectors for Intranuclear DNA Delivery,” Bioorg. Med. Chem. Lett. 27:4781-4785 (2017), which are hereby incorporated by reference in their entirety). For example, Liedl et al. enhanced gene expression in DNA nanoparticles by incorporating DNA nuclear targeting sequences (DTS) (Liedl et al., “Active Nuclear Import of Mammalian Cell-Expressible DNA Origami,” J. Am. Chem. Soc. 145:4946-4950 (2023), which is hereby incorporated by reference in its entirety). In summary, self-assembling DNA nanoparticles hold great potential as a platform for targeted in vivo gene expression and gene modification.

This study demonstrates the effective transcription of self-assembling DNA nanoparticles in human cell culture. In contrast to prior findings, where increased flexibility by increasing the portion of ssDNA domains enhanced transcription efficiency in a nuclease-free test tube system, in cell culture an inverse relationship between gene expression and degree of exposed ssDNA domains was observed (Oh and Henderson, “In Vitro Transcription of Self-Assembling DNA Nanoparticles,” Sci. Rep. 13(1): 12961 (2023), which is hereby incorporated by reference in its entirety). This can be attributed to the increased susceptibility of single-stranded DNA (ssDNA) to degradation by cellular nucleases compared to double-stranded DNA (dsDNA). Further, the position of local ssDNA domains within the DNA nanoparticles appears to influence gene expression, with ssDNA domains positioned near the exterior of the architecture having greater susceptibility to single-strand-specific nuclease attack, resulting in a reduction in gene expression.

Additionally, it was found that the collective folding of the gene with the promoter/enhancer elements resulted in decreased gene expression compared to nanoparticles with linear promoter and enhancer configurations. Interestingly, regardless of the compactness of the promoter/enhancer folding, the level of gene expression among nanoparticle samples with the folded promoter/enhancer and gene remained comparable, but perhaps surprisingly, nanoparticles with tightly folded promoter and enhancer domains exhibited a slight increase in gene expression, presumably due to enhanced nuclease resistance resulting from their slightly more compact architecture. Combined with previous research, these findings emphasize the potential of gene-bearing, self-assembling DNA nanoparticles containing nuanced architectural features in serving as a platform in biomedicine and related fields. Of further interest was the observation that when the DNA nanoparticle carrier solution was switched from 1×TEAM to 1×PBSM, the level of gene expression increased noticeably for the architectures in which the promoter, enhancer, and protein coding regions were folded altogether, introducing another potential variable for consideration in the fine tuning of DNA nanoparticles for desired efficacy as therapeutic agents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A nucleic acid nanoparticle comprising:
a single stranded nucleic acid scaffold encoding one or more RNA molecules and/or proteins of interest and
a plurality of single stranded nucleic acid oligomers, wherein each oligomer is complementary to at least a portion of the single stranded nucleic acid scaffold.

2. The nucleic acid nanoparticle according to claim 1, wherein the nucleic acid scaffold is a DNA molecule.

3. The nucleic acid nanoparticle according to claim 1, wherein the nucleic acid scaffold encodes one or more RNA molecules.

4. The nucleic acid nanoparticle according to claim 1, wherein the single stranded nucleic acid scaffold encodes one or more protein(s) of interest.

5. The nucleic acid nanoparticle according to claim 4, wherein the one or more proteins of interest comprise one or more antigens.

6. The nucleic acid nanoparticle according to claim 5, wherein the one or more antigens is derived from a pathogenic microorganism; a tumor-associated antigen;
and an allergen.

7. The nucleic acid nanoparticle according to claim 1, wherein the single stranded nucleic acid scaffold encodes a gene-editing system.

8. The nucleic acid nanoparticle according to claim 7, wherein the gene-editing system is selected from the group consisting of a Clustered Regularly Interspaced Short Palindromic Repeat/Clustered Regularly Interspaced Short Palindromic Repeat associated (CRISPR/Cas) system, a zinc finger nuclease (“ZFNs”), or a transcription activator-like effector nucleases (“TALEN”).

9. The nucleic acid nanoparticle according to claim 1, wherein the single stranded nucleic acid scaffold and/or the plurality of single stranded nucleic acid oligomers comprise a cationic lipid moiety.

10. The nucleic acid nanoparticle according to claim 9, wherein the cationic lipid moiety is selected from the group consisting of 1,2-DiLinoleyloxy-N,N-dimethylaminopropane (DLinDMA) and 1,2-Dilinolenyloxy-N,N-dimethylaminopropane (DLenDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3—(N—(N′,N′-dimethylaminoethane)-carbamoyl)cholesterol (DC-Chol); N-(1,2-dimyristyloxyprop-3-yl)-N,N-dimethyl-N-hydroxyethyl ammonium bromide (DMRIE); and derivatives thereof.

11. The nucleic acid nanoparticle according to claim 1, wherein the single stranded nucleic acid scaffold and/or the plurality of single stranded nucleic acid oligomers comprise a targeting peptide sequence.

12. The nucleic acid nanoparticle according to claim 11, wherein the targeting peptide sequence is selected from the group consisting of a cell penetrating peptide (CPP) and a nuclear localization signal (NLS).

13. The nucleic acid nanoparticle according to claim 12, wherein the targeting peptide sequence is a cell penetrating peptide (CPP) selected from the group consisting of HIV-1 Trans-Activator of Transcription (Tat) Peptide, Syn-B1, Syn B-3, Poly-L-arginine, penetratin (43-58), amphipathic model peptide, transportin, SBP, and FBP.

14. The nucleic acid nanoparticle according to claim 12, wherein the targeting peptide sequence is a nuclear localization signal selected from the group consisting of V40 Large T-antigen, VACM-1/CUL5, CXCR4, VP1, 53BP1, ING4, IER5, ERK5, Hrp1, UL79, EWS, PtHrP, Pho4, rpL23a, MSX1, NLS-RARα, bipartite nucleoplasmin NLS, and derivatives thereof.

15. The nucleic acid nanoparticle according to claim 1, wherein the single stranded nucleic acid scaffold and/or the plurality of single stranded nucleic acid oligomers comprise one or more biodegradable cationic polymers.

16. The nucleic acid nanoparticle according to claim 15, wherein the biodegradable cationic polymers are polyethylene glycerol (PEG)-poly-lysine copolymers.

17. The nucleic acid nanoparticle according to claim 1, wherein the single stranded nucleic acid scaffold and/or the plurality of single stranded nucleic acid oligomers comprise one or more peptoids.

18-20. (canceled)

21. A pharmaceutical composition comprising the nucleic acid nanoparticle according to claim 1, and a pharmaceutically acceptable carrier.

22. A method of eliciting an immune response in a subject, said method comprising:
administering a nucleic acid nanoparticle according to claim 1, to a subject, wherein the nucleic acid nanoparticle is administered to the subject to elicit an immune response.

23-31. (canceled)

32. A method of expressing a heterologous RNA molecule and/or protein of interest in a population of cells, said method comprising:
contacting a population of cells with a nucleic acid nanoparticle according to claim 1.

33-44. (canceled)


SEQUENCE LISTING (217 sequences)

SEQ ID NO: 1 - protein, 13 aa, synthetic construct
  REGION 1..13: note = HIV Tat peptide (48-60)
GRKKRRQRRRPPQ

SEQ ID NO: 2 - protein, 18 aa, synthetic construct
  REGION 1..18: note = Syn-B1
RGGRLSYSRRRFSTSTGR

SEQ ID NO: 3 - protein, 10 aa, synthetic construct
  REGION 1..10: note = Syn-B3
RRLSYSRRRF

SEQ ID NO: 4 - protein, 7 aa, synthetic construct
  REGION 1..7: note = Poly-L-arginine
RRRRRRR

SEQ ID NO: 5 - protein, 9 aa, synthetic construct
  REGION 1..9: note = Poly-L-arginine
RRRRRRRRR

SEQ ID NO: 6 - protein, 16 aa, synthetic construct
  REGION 1..16: note = penetratin (43-58)
RQIKIWFQNRRMKWKK

SEQ ID NO: 7 - protein, 18 aa, synthetic construct
  REGION 1..18: note = amphipathic model peptide
KLALKLALKALKAALKLA

SEQ ID NO: 8 - protein, 26 aa, synthetic construct
  REGION 1..26: note = transportin
GWTLNSAGYLLKINLKALAALAKKIL

SEQ ID NO: 9 - protein, 27 aa, synthetic construct
  REGION 1..27: note = SBP
MGLGLHLLVLAAALQGAWSQPKKKRKV

SEQ ID NO: 10 - protein, 27 aa, synthetic construct
  REGION 1..27: note = FBP
GALFLGWLGAAGSTMGAWSQPKKKRKV

SEQ ID NO: 11 - protein, 7 aa, synthetic construct
  REGION 1..7: note = SV40 Large T-antigen
PKKKRKV

SEQ ID NO: 12 - protein, 6 aa, synthetic construct
  REGION 1..6: note = VACM-1/CUL5
PKLKRQ

SEQ ID NO: 13 - protein, 4 aa, synthetic construct
  REGION 1..4: note = CXCR4
RPRK

SEQ ID NO: 14 - protein, 8 aa, synthetic construct
  REGION 1..8: note = VP1
RRARRPRG

SEQ ID NO: 15 - protein, 21 aa, synthetic construct
  REGION 1..21: note = 53BP1
GKRKLITSEEERSPAKRGRKS

SEQ ID NO: 16 - protein, 22 aa, synthetic construct
  REGION 1..22: note = ING4
KGKKGRTQKEKKAARARSKGKN

SEQ ID NO: 17 - protein, 28 aa, synthetic construct
  REGION 1..28: note = IER5
RKRCAAGVGGGPAGCPAPGSTPLKKPRR

SEQ ID NO: 18 - protein, 36 aa, synthetic construct
  REGION 1..36: note = ERK5
RKPVTAQERQREREEKRRRRQERAKEREKRRQERER

SEQ ID NO: 19 - protein, 27 aa, synthetic construct
  REGION 1..27: note = Hrp1
RSGGNHRRNGRGGRGGYNRRNNGYHPY

SEQ ID NO: 20 - protein, 27 aa, synthetic construct
  REGION 1..27: note = UL79
TLLLRETMNNLGVSDHAVLSRKTPQPY

SEQ ID NO: 21 - protein, 18 aa, synthetic construct
  REGION 1..18: note = EWS
PGKMDKGEHRQERRDRPY

SEQ ID NO: 22 - protein, 21 aa, synthetic construct
  REGION 1..21: note = parathyroid hormone-related protein (PtHrP)
GKKKKGKPGKRREQRKKKRRT

SEQ ID NO: 23 - protein, 27 aa, synthetic construct
  REGION 1..27: note = Pho4
SANKVTKNKSNSSPYLNKRKGKPGPDS

SEQ ID NO: 24 - protein, 43 aa, synthetic construct
  REGION 1..43: note = rpL23a
VHSHKKKKIPTSPTFTTPKTLTLRRQPKYPRKSAPRRNKLDHY

SEQ ID NO: 25 - protein, 10 aa, synthetic construct
  REGION 1..10: note = MSX1
RKHKTNRKPR

SEQ ID NO: 26 - protein, 8 aa, synthetic construct
  REGION 1..8: note = MSX1
NRRAKAKR

SEQ ID NO: 27 - protein, 6 aa, synthetic construct
  REGION 1..6: note = NLS-RAR alpha
RNKKKK

SEQ ID NO: 28 - protein, 5 aa, synthetic construct
  REGION 1..5: note = NLS-RAR alpha
RKVIK

SEQ ID NO: 29 - protein, 16 aa, synthetic construct
  REGION 1..16: note = Bipartite nucleoplasmin NLS (155-170)
KRPAAIKKAGQAKKKK

SEQ ID NO: 30 - DNA, 30 bp, synthetic construct
  source 1..30: mol_type = other DNA
  misc_feature 1..30: note = T7EGFP sense primer
ccagatatacgcgttgacattgattattga

SEQ ID NO: 31 - DNA, 32 bp, synthetic construct
  source 1..32: mol_type = other DNA
  misc_feature 1..32: note = T7EGFP anti primer
tatatacaatttcacacaggaaacagctatga

SEQ ID NO: 32 - DNA, 30 bp, synthetic construct; identical to SEQ ID NO: 30
  source 1..30: mol_type = other DNA
  misc_feature 1..30: note = 3-prime T7EGFP blocker
  modified_base 30: mod_base = OTHER; note = PHOSPHORYLATION

SEQ ID NO: 33 - DNA, 18 bp, synthetic construct
  source 1..18: mol_type = other DNA
  misc_feature 1..18: note = RT sense primer
tggtgagcaagggcgagg

SEQ ID NO: 34 - DNA, 24 bp, synthetic construct
  source 1..24: mol_type = other DNA
  misc_feature 1..24: note = RT anti primer
tcacttgtacagctcgtccatgcc

SEQ ID NO: 35 - DNA, 35 bp, synthetic construct
  source 1..35: mol_type = other DNA
  misc_feature 1..35: note = GHL 1
aagctggatccgcccctgagcacccagtgttcgtg

SEQ ID NO: 36 - DNA, 42 bp, synthetic construct
  source 1..42: mol_type = other DNA
  misc_feature 1..42: note = GHL 2
agttccgcgttacataacttacggtaaatggcccgcctggct

SEQ ID NO: 37 - DNA, 28 bp, synthetic construct
  source 1..28: mol_type = other DNA
  misc_feature 1..28: note = GHL 3
gctgcccccaccctcgtgaccgggcgac

SEQ ID NO: 38 - DNA, 35 bp, synthetic construct
  source 1..35: mol_type = other DNA
  misc_feature 1..35: note = GHL 4
aggtgccgccatcttgctggaccgccctaggtgaa

SEQ ID NO: 39 - DNA, 42 bp, synthetic construct
  source 1..42: mol_type = other DNA
  misc_feature 1..42: note = GHL 5
atgggtggagtatttacggtaaactgcccacttggcagtaca

SEQ ID NO: 40 - DNA, 25 bp, synthetic construct
  source 1..25: mol_type = other DNA
  misc_feature 1..25: note = GHL 6
cggtaggcgtgtacggtgggaggtc

SEQ ID NO: 41 - DNA, 35 bp, synthetic construct
  source 1..35: mol_type = other DNA
  misc_feature 1..35: note = GHL 7
cgccgactcgacttagttcgaaccctgatgcacca

SEQ ID NO: 42 - DNA, 36 bp, synthetic construct
  source 1..36: mol_type = other DNA
  misc_feature 1..36: note = GHL 8
atcgctattaccatggtgatgcggttttggcagtac

SEQ ID NO: 43 - DNA, 35 bp, synthetic construct
  source 1..35: mol_type = other DNA
  misc_feature 1..35: note = GHL 9
cgaggacacatcctacaagactgcttcaaagctga

SEQ ID NO: 44 - DNA, 49 bp, synthetic construct
  source 1..49: mol_type = other DNA
  misc_feature 1..49: note = GHL 10
aggacagctgtttcctgtgtggcaagggcgaggaggttcatccctacgg

SEQ ID NO: 45 - DNA, 42 bp, synthetic construct
  source 1..42: mol_type = other DNA
  misc_feature 1..42: note = GHL 11
ccagatatacgcgttgacattgattattgactagttattaat

SEQ ID NO: 46 - DNA, 28 bp, synthetic construct
  source 1..28: mol_type = other DNA
  misc_feature 1..28: note = GHL 12
ccccgacggacgacggcaactggggcac

SEQ ID NO: 47 - DNA, 42 bp, synthetic construct
  source 1..42: mol_type = other DNA
  misc_feature 1..42: note = GHL 13
tgttcccatagtaacgccaatagggactttccattgacgtca

SEQ ID NO: 48 - DNA, 35 bp, synthetic construct
  source 1..35: mol_type = other DNA
  misc_feature 1..35: note = GHL 14
aatcatgccggggtggtgcccctacggcgccgcta

SEQ ID NO: 49 - DNA, 35 bp, synthetic construct
  source 1..35: mol_type = other DNA
  misc_feature 1..35: note = GHL 15
cttcaagagtacaatcttcaacacatgagagggcg

SEQ ID NO: 50 - DNA, 16 bp, synthetic construct
  source 1..16: mol_type = other DNA
  misc_feature 1..16: note = GHL 16
gaaggcttatcatggc

SEQ ID NO: 51 - DNA, 42 bp, synthetic construct
  source 1..42: mol_type = other DNA
  misc_feature 1..42: note = GHL 17
tatataagcagagctggtttagtgaaccgtcagatccgctag

SEQ ID NO: 52 - DNA, 35 bp, synthetic construct
  source 1..35: mol_type = other DNA
  misc_feature 1..35: note = GHL 18
atgccacatcctggctagagccaatagcctgtcct

SEQ ID NO: 53 - DNA, 35 bp, synthetic construct
  source 1..35: mol_type = other DNA
  misc_feature 1..35: note = GHL 19
cctctagtcgagctggacggccgagggcagcagca

SEQ ID NO: 54 - DNA, 35 bp, synthetic construct
  source 1..35: mol_type = other DNA
  misc_feature 1..35: note = GHL 20
ttcctaagactgtgccgggataccactaacaacat

SEQ ID NO: 55 - DNA, 35 bp, synthetic construct
  source 1..35: mol_type = other DNA
  misc_feature 1..35: note = GHL 21
tgtccgggacgtaactcgatatgcggtgccctgga

SEQ ID NO: 56 - DNA, 42 bp, synthetic construct
  source 1..42: mol_type = other DNA
  misc_feature 1..42: note = GHL 22
agtaatcaattacggggtcattagttcatagcccatatatgg

SEQ ID NO: 57 - DNA, 49 bp, synthetic construct
  source 1..49: mol_type = other DNA
  misc_feature 1..49: note = GHL 23
agccacaggcatcagagcaaagaccccacgatcacatggtccgttgttt

SEQ ID NO: 58 - DNA, 43 bp, synthetic construct
  source 1..43: mol_type = other DNA
  misc_feature 1..43: note = GHL 24
gaccgcccaacgacccccgcccaatgacgtcaataatgacgta

SEQ ID NO: 59 - DNA, 23 bp, synthetic construct
  source 1..23: mol_type = other DNA
  misc_feature 1..23: note = GHL 25
agaaccagctggggacggccaca

SEQ ID NO: 60 - DNA, 46 bp, synthetic construct
  source 1..46: mol_type = other DNA
  misc_feature 1..46: note = GHL 26
agatccgcggccgctaatacgactcactatagggagagccgccacc

SEQ ID NO: 61 - DNA, 43 bp, synthetic construct
  source 1..43: mol_type = other DNA
  misc_feature 1..43: note = GHL 27
tcaagtgtatcatatgccaagtacgccccctattgacgtcaat

SEQ ID NO: 62 - DNA, 35 bp, synthetic construct
  source 1..35: mol_type = other DNA
  misc_feature 1..35: note = GHL 28
gacggcaggcagcggctgctgcccgacacactctc

SEQ ID NO: 63 - DNA, 42 bp, synthetic construct
  source 1..42: mol_type = other DNA
  misc_feature 1..42: note = GHL 29
agtctccaccccattgacgtcaatgggagtttgttttggcac

SEQ ID NO: 64 - DNA, 28 bp, synthetic construct
  source 1..28: mol_type = other DNA
  misc_feature 1..28: note = GHL 30
cgtgcagccgcgccgaggtgacaaggag

SEQ ID NO: 65 - DNA, 42 bp, synthetic construct
  source 1..42: mol_type = other DNA
  misc_feature 1..42: note = GHL 31
acaagcagaagaacacgtctaacgtccattcaagtttcagcg

SEQ ID NO: 66 - DNA, 42 bp, synthetic construct
  source 1..42: mol_type = other DNA
  misc_feature 1..42: note = GHL 32
gacggtaaatggcccgcctggcattatgcccagtacatgacc

SEQ ID NO: 67 - DNA, 42 bp, synthetic construct
  source 1..42: mol_type = other DNA
  misc_feature 1..42: note = GHL 33
atcaatgggcgtggatagcggtttgactcacggggatttcca

SEQ ID NO: 68 - DNA, 42 bp, synthetic construct
  source 1..42: mol_type = other DNA
  misc_feature 1..42: note = GHL 34
ttatgggactttcctacttggcagtacatctacgtattagtc

SEQ ID NO: 69 - DNA, 17 bp, synthetic construct
  source 1..17: mol_type = other DNA
  misc_feature 1..17: note = GHL 35
ccattgacgcaaatggg

SEQ ID NO: 70 - DNA, 35 bp, synthetic construct
  source 1..35: mol_type = other DNA
  misc_feature 1..35: note = GHL 36
ccctgaactgttcagtcatagcaaggggaggaaat

SEQ ID NO: 71 - DNA, 35 bp, synthetic construct
  source 1..35: mol_type = other DNA
  misc_feature 1..35: note = GHL 37
tgcatcgttaaaccacgagctgccccgttgcagct

SEQ ID NO: 72 - DNA, 28 bp, synthetic construct
  source 1..28: mol_type = other DNA
  misc_feature 1..28: note = GHL 38
cgacttcggagcgcaccatctctacaac

SEQ ID NO: 73 - DNA, 49 bp, synthetic construct
  source 1..49: mol_type = other DNA
  misc_feature 1..49: note = GHL 39
ggcatggcgctgatcagcctctaaaatggaggattgggaagattggcgt

SEQ ID NO: 74 - DNA, 49 bp, synthetic construct
  source 1..49: mol_type = other DNA
  misc_feature 1..49: note = GHL 40
accctggcgagctgaagggcacactaccccccatcggcgacggtacaag

SEQ ID NO: 75 - DNA, 33 bp, synthetic construct
  source 1..33: mol_type = other DNA
  misc_feature 1..33: note = GHL 41
gcccctctccttgaggctctatggcttctgagg

SEQ ID NO: 76 - DNA, 42 bp, synthetic construct
  source 1..42: mol_type = other DNA
  misc_feature 1..42: note = GHL 42
caaaatcaacgggactttccaaaatgtcgtaacaactccgcc

SEQ ID NO: 77 - DNA, 35 bp, synthetic construct
  source 1..35: mol_type = other DNA
  misc_feature 1..35: note = GHL 43
tgaccggcatcaccattgagtcattgtcgtggggc

SEQ ID NO: 78 - DNA, 49 bp, synthetic construct
  source 1..49: mol_type = other DNA
  misc_feature 1..49: note = GHL 44
accgccgccttctagttgccaactcccaaggcatgctggggaccgtcga

SEQ ID NO: 79 - DNA, 42 bp, synthetic construct
  source 1..42: mol_type = other DNA
  misc_feature 1..42: note = GHL 45
ccggcaaatggtgaaaattgtgggtgggtgagtaggtgtcat

SEQ ID NO: 80 - DNA, 35 bp, synthetic construct
  source 1..35: mol_type = other DNA
  misc_feature 1..35: note = GHL BP 1
gcagcactgtccgggacgtaactcgatatgcggtg

SEQ ID NO: 81 - DNA, 21 bp, synthetic construct
  source 1..21: mol_type = other DNA
  misc_feature 1..21: note = GHL BP 2
aagctgagtgcagtggacgac

SEQ ID NO: 82 - DNA, 35 bp, synthetic construct
  source 1..35: mol_type = other DNA
  misc_feature 1..35: note = GHL BP 3
ctgtgtggcaagggatagggaagggcatggcagcg

SEQ ID NO: 83 - DNA, 43 bp, synthetic construct
  source 1..43: mol_type = other DNA
  misc_feature 1..43: note = GHL BP 4
ccaagtctccaccccattgacgtcaatgggagtttgttttggc

SEQ ID NO: 84 - DNA, 21 bp, synthetic construct
  source 1..21: mol_type = other DNA
  misc_feature 1..21: note = GHL BP 5
tgcaccagtgaccaccgcgcc

SEQ ID NO: 85 - DNA, 46 bp, synthetic construct
  source 1..46: mol_type = other DNA
  misc_feature 1..46: note = GHL BP 6
gtaacgccaatagggactttccattgacgtcaatgggtggagtatt

SEQ ID NO: 86 - DNA, 35 bp, synthetic construct
  source 1..35: mol_type = other DNA
  misc_feature 1..35: note = GHL BP 7
gaggatttcctaatactgtgccgggatcccactac

SEQ ID NO: 87 - DNA, 46 bp, synthetic construct
  source 1..46: mol_type = other DNA
  misc_feature 1..46: note = GHL BP 8
acatctacgtattagtcatcgctattaccatggtgatgcggttttg

SEQ ID NO: 88 - DNA, 47 bp, synthetic construct
  source 1..47: mol_type = other DNA
  misc_feature 1..47: note = GHL BP 9
accaaaatcaacgggactttccaaaatgtcgtaacaactccgcccca

SEQ ID NO: 89 - DNA, 35 bp, synthetic construct
  source 1..35: mol_type = other DNA
  misc_feature 1..35: note = GHL BP 10
ggcaactaaggaggtaatacgagcctcgaaaatga

SEQ ID NO: 90 - DNA, 35 bp, synthetic construct
  source 1..35: mol_type = other DNA
  misc_feature 1..35: note = GHL BP 11
caccctcccggcaaatggtgaaaattgtgggtggg

SEQ ID NO: 91 - DNA, 46 bp, synthetic construct
  source 1..46: mol_type = other DNA
  misc_feature 1..46: note = GHL BP 12
aagtacgccccctattgacgtcaatgacggtaaatggcccgcctgg

SEQ ID NO: 92 - DNA, 35 bp, synthetic construct
  source 1..35: mol_type = other DNA
  misc_feature 1..35: note = GHL BP 13
gtggggcgcatcgctaaaccccgagctgccccgtg

SEQ ID NO: 93 - DNA, 35 bp, synthetic construct
  source 1..35: mol_type = other DNA
  misc_feature 1..35: note = GHL BP 14
gcatggagctgatcactcactcgaggaggttcatc

SEQ ID NO: 94 - DNA, 21 bp, synthetic construct
  source 1..21: mol_type = other DNA
  misc_feature 1..21: note = GHL BP 15
ggaaattaggacagctgtttc

SEQ ID NO: 95 - DNA, 46 bp, synthetic construct
  source 1..46: mol_type = other DNA
  misc_feature 1..46: note = GHL BP 16
ttgacgcaaatgggcggtaggcgtgtacggtgggaggtctatataa

SEQ ID NO: 96 - DNA, 21 bp, synthetic construct
  source 1..21: mol_type = other DNA
  misc_feature 1..21: note = GHL BP 17
gagggcgcccgaccggagcgc

SEQ ID NO: 97 - DNA, 43 bp, synthetic construct
  source 1..43: mol_type = other DNA
  misc_feature 1..43: note = GHL BP 18
agtaatcaattacggggtcattagttcatagcccatatatgga

SEQ ID NO: 98 - DNA, 21 bp, synthetic construct
  source 1..21: mol_type = other DNA
  misc_feature 1..21: note = GHL BP 19
cctggaactggggaccgtcga

SEQ ID NO: 99 - DNA, 42 bp, synthetic construct; identical to SEQ ID NO: 45
  source 1..42: mol_type = other DNA
  misc_feature 1..42: note = GHL BP 20

SEQ ID NO: 100 - DNA, 35 bp, synthetic construct
  source 1..35: mol_type = other DNA
  misc_feature 1..35: note = GHL BP 21
tggtcctttgtttggtgaaccggacggccgagggc

SEQ ID NO: 101 - DNA, 35 bp, synthetic construct
  source 1..35: mol_type = other DNA
  misc_feature 1..35: note = GHL BP 22
ccgctacatgccacatcctggctagagccaatagc

SEQ ID NO: 102 - DNA, 35 bp, synthetic construct
  source 1..35: mol_type = other DNA
  misc_feature 1..35: note = GHL BP 23
gaaggcttacaacatggtttacccctccccttgac

SEQ ID NO: 103 - DNA, 18 bp, synthetic construct
  source 1..18: mol_type = other DNA
  misc_feature 1..18: note = GHL BP 24
cctggtgaacactaccag

SEQ ID NO: 104 - DNA, 35 bp, synthetic construct
  source 1..35: mol_type = other DNA
  misc_feature 1..35: note = GHL BP 25
ccgccgccttctagagatccgggtgcccctacggc

SEQ ID NO: 105 - DNA, 47 bp, synthetic construct
  source 1..47: mol_type = other DNA
  misc_feature 1..47: note = GHL BP 26
gttccgcgttacataacttacggtaaatggcccgcctggctgaccgc

SEQ ID NO: 106 - DNA, 35 bp, synthetic construct
  source 1..35: mol_type = other DNA
  misc_feature 1..35: note = GHL BP 27
ctgagcacttcaagcatcctgtcttcaagcttcag

SEQ ID NO: 107 - DNA, 21 bp, synthetic construct
  source 1..21: mol_type = other DNA
  misc_feature 1..21: note = GHL BP 28
acaacatccgacaaactctcg

SEQ ID NO: 108 - DNA, 35 bp, synthetic construct
  source 1..35: mol_type = other DNA
  misc_feature 1..35: note = GHL BP 29
cctctagtcgagctgtcagatagctggaggcatca

SEQ ID NO: 109 - DNA, 35 bp, synthetic construct
  source 1..35: mol_type = other DNA
  misc_feature 1..35: note = GHL BP 30
aatcatgccggggtcggccgcacggcaaatccgcc

SEQ ID NO: 110 - DNA, 35 bp, synthetic construct
  source 1..35: mol_type = other DNA
  misc_feature 1..35: note = GHL BP 31
ctacggcccctgaactgttcagtcatagcaagggg

SEQ ID NO: 111 - DNA, 21 bp, synthetic construct
  source 1..21: mol_type = other DNA
  misc_feature 1..21: note = GHL BP 32
ttcagcggacttctcatgccc

SEQ ID NO: 112 - DNA, 21 bp, synthetic construct
  source 1..21: mol_type = other DNA
  misc_feature 1..21: note = GHL BP 33
ggctctatggcttcagaacca

SEQ ID NO: 113 - DNA, 46 bp, synthetic construct
  source 1..46: mol_type = other DNA
  misc_feature 1..46: note = GHL BP 34
gcagtacatcaatgggcgtggatagcggtttgactcacggggattt

SEQ ID NO: 114 - DNA, 42 bp, synthetic construct
  source 1..42: mol_type = other DNA
  misc_feature 1..42: note = GHL BP 35
gaacacccccatcgcgccgacccgcatcagttcgacctggcc

SEQ ID NO: 115 - DNA, 47 bp, synthetic construct
  source 1..47: mol_type = other DNA
  misc_feature 1..47: note = GHL BP 36
ccaacgacccccgcccaatgacgtcaataatgacgtatgttcccata

SEQ ID NO: 116 - DNA, 21 bp, synthetic construct
  source 1..21: mol_type = other DNA
  misc_feature 1..21: note = GHL BP 37
tgtccttgggaagattggcgt

SEQ ID NO: 117 - DNA, 24 bp, synthetic construct
  source 1..24: mol_type = other DNA
  misc_feature 1..24: note = GHL BP 38
gaccggtcatcatcaccatcacca

SEQ ID NO: 118 - DNA, 35 bp, synthetic construct
  source 1..35: mol_type = other DNA
  misc_feature 1..35: note = GHL BP 39
ctgctgccgaggaccgacttcacaagacccctgac

SEQ ID NO: 119 - DNA, 46 bp, synthetic construct
  source 1..46: mol_type = other DNA
  misc_feature 1..46: note = GHL BP 40
cattatgcccagtacatgaccttatgggactttcctacttggcagt

SEQ ID NO: 120 - DNA, 35 bp, synthetic construct
  source 1..35: mol_type = other DNA
  misc_feature 1..35: note = GHL BP 41
agcaaaggaagaacgtacaacacgtccaacatgaa

SEQ ID NO: 121 - DNA, 35 bp, synthetic construct
  source 1..35: mol_type = other DNA
  misc_feature 1..35: note = GHL BP 42
accatctgggcacaccgctagttgccagctcccac

SEQ ID NO: 122 - DNA, 49 bp, synthetic construct
  source 1..49: mol_type = other DNA
  misc_feature 1..49: note = GHL BP 43
gaggtgagagctgagagccgcttgagttattgtctgagtaggttctggg

SEQ ID NO: 123 - DNA, 46 bp, synthetic construct
  source 1..46: mol_type = other DNA
  misc_feature 1..46: note = GHL BP 44
tacggtaaactgcccacttggcagtacatcaagtgtatcatatgcc

SEQ ID NO: 124 - DNA, 35 bp, synthetic construct
  source 1..35: mol_type = other DNA
  misc_feature 1..35: note = GHL BP 45
gctggggacggccagcagagcgccacaaatggccg

SEQ ID NO: 125 - DNA, 35 bp, synthetic construct
  source 1..35: mol_type = other DNA
  misc_feature 1..35: note = GHL BP 46
aggcatgggtgccaccatctggctggagcgccctg

SEQ ID NO: 126 - DNA, 21 bp, synthetic construct
  source 1..21: mol_type = other DNA
  misc_feature 1..21: note = GHL BP 47
tgcagctgcgacggtacaagt

SEQ ID NO: 127 - DNA, 21 bp, synthetic construct
  source 1..21: mol_type = other DNA
  misc_feature 1..21: note = GHL BP 48
acaagcaaccccaagatcaca

SEQ ID NO: 128 - DNA, 21 bp, synthetic construct
  source 1..21: mol_type = other DNA
  misc_feature 1..21: note = GHL BP 49
aggtgaacccagtcttcgtga

SEQ ID NO: 129 - DNA, 32 bp, synthetic construct
  source 1..32: mol_type = other DNA
  misc_feature 1..32: note = GHL FSC 1
tattctggggggtgggtgagtaggtgtcattc

SEQ ID NO: 130 - DNA, 50 bp, synthetic construct
  source 1..50: mol_type = other DNA
  misc_feature 1..50: note = GHL FSC 2
gctgcccgtgccctggcccaccctcgtgaccgggcgacaccctggtgaac

SEQ ID NO: 131 - DNA, 38 bp, synthetic construct
  source 1..38: mol_type = other DNA
  misc_feature 1..38: note = GHL FSC 3
aagcgcgatcacatggtccgttgtttgcccctcccccg

SEQ ID NO: 132 - DNA, 18 bp, synthetic construct
  source 1..18: mol_type = other DNA
  misc_feature 1..18: note = GHL FSC 4
ccgaaggcttatcatggc

SEQ ID NO: 133 - DNA, 44 bp, synthetic construct
  source 1..44: mol_type = other DNA
  misc_feature 1..44: note = GHL FSC 5
agttcagcgtgtccgggacgtaactcgatatgcggtgccctgga

SEQ ID NO: 134 - DNA, 31 bp, synthetic construct
  source 1..31: mol_type = other DNA
  misc_feature 1..31: note = GHL FSC 6
cgcatcgagctgaagggcacactaccagcag

SEQ ID NO: 135 - DNA, 33 bp, synthetic construct
  source 1..33: mol_type = other DNA
  misc_feature 1..33: note = GHL FSC 7
agccacaggcatcagagcaaagaccccaacgag

SEQ ID NO: 136 - DNA, 28 bp, synthetic construct
  source 1..28: mol_type = other DNA
  misc_feature 1..28: note = GHL FSC 8
cggaaagaaccagctggggacggccaca

SEQ ID NO: 137 - DNA, 26 bp, synthetic construct
  source 1..26: mol_type = other DNA
  misc_feature 1..26: note = GHL FSC 9
ccggcaaatggtgaaaattgtatata

SEQ ID NO: 138 - DNA, 46 bp, synthetic construct
  source 1..46: mol_type = other DNA
  misc_feature 1..46: note = GHL FSC 10
cgacaagcagaagaacacgtctaacgtccattcaagtccgccatgc

SEQ ID NO: 139 - DNA, 38 bp, synthetic construct
  source 1..38: mol_type = other DNA
  misc_feature 1..38: note = GHL FSC 11
aacacccccatcggcgacggtacaagtgaccggtcatc

SEQ ID NO: 140 - DNA, 31 bp, synthetic construct
  source 1..31: mol_type = other DNA
  misc_feature 1..31: note = GHL FSC 12
tgccttccttgaggctctatggcttctgagg

SEQ ID NO: 141 - DNA, 33 bp, synthetic construct
  source 1..33: mol_type = other DNA
  misc_feature 1..33: note = GHL FSC 13
atcaccatcaccattgagtcattgtcgtggggc

SEQ ID NO: 142 - DNA, 27 bp, synthetic construct
  source 1..27: mol_type = other DNA
  misc_feature 1..27: note = GHH 1
cgggactttccaaaatgtcgtaacaac

SEQ ID NO: 143 - DNA, 29 bp, synthetic construct
  source 1..29: mol_type = other DNA
  misc_feature 1..29: note = GHH 2
tgacgtcaataatgacgtatgttcccata

SEQ ID NO: 144 - DNA, 32 bp, synthetic construct
  source 1..32: mol_type = other DNA
  misc_feature 1..32: note = GHH 3
cagtacatcaatgggcggtgatgcggttttgg

SEQ ID NO: 145 - DNA, 28 bp, synthetic construct
  source 1..28: mol_type = other DNA
  misc_feature 1..28: note = GHH 4
catgaccttatgggactttcccccgcat

SEQ ID NO: 146 - DNA, 28 bp, synthetic construct
  source 1..28: mol_type = other DNA
  misc_feature 1..28: note = GHH 5
ccgcggcgacattgccatatatggagtt

SEQ ID NO: 147 - DNA, 37 bp, synthetic construct
  source 1..37: mol_type = other DNA
  misc_feature 1..37: note = GHH 6
tggcatcagtacatcaagtgcaatagggactttccat

SEQ ID NO: 148 - DNA, 31 bp, synthetic construct
  source 1..31: mol_type = other DNA
  misc_feature 1..31: note = GHH 7
tattgacgtcaatgacggtaaatggcccgcc

SEQ ID NO: 149 - DNA, 30 bp, synthetic construct
  source 1..30: mol_type = other DNA
  misc_feature 1..30: note = GHH 8
gtaacgctatcatatgccaagtacgccccc

SEQ ID NO: 150 - DNA, 44 bp, synthetic construct
  source 1..44: mol_type = other DNA
  misc_feature 1..44: note = GHH 9
atgggcggtaggcgtgtacggtgactatagggagagccgccacc

SEQ ID NO: 151 - DNA, 37 bp, synthetic construct
  source 1..37: mol_type = other DNA
  misc_feature 1..37: note = GHH 10
tgaacattgacgtcaatgggagtttgttttgacgcaa

SEQ ID NO: 152 - DNA, 35 bp, synthetic construct
  source 1..35: mol_type = other DNA
  misc_feature 1..35: note = GHH 11
ccgcgttcagatatacgcgttcgctaatacgactc

SEQ ID NO: 153 - DNA, 39 bp, synthetic construct
  source 1..39: mol_type = other DNA
  misc_feature 1..39: note = GHH 12
tcatctttacggtaaactgcccacttggtatgcccagta

SEQ ID NO: 154 - DNA, 25 bp, synthetic construct
  source 1..25: mol_type = other DNA
  misc_feature 1..25: note = GHH 13
tccgccccattggcaccaaaatcaa

SEQ ID NO: 155 - DNA, 30 bp, synthetic construct
  source 1..30: mol_type = other DNA
  misc_feature 1..30: note = GHH 14
ttgactcacggggatttccaagtctccacc

SEQ ID NO: 156 - DNA, 25 bp, synthetic construct
  source 1..25: mol_type = other DNA
  misc_feature 1..25: note = GHH 15
tacttggcagtacatctacgtatta

SEQ ID NO: 157 - DNA, 25 bp, synthetic construct
  source 1..25: mol_type = other DNA
  misc_feature 1..25: note = GHH 16
tgacgtcaatgggtggagtaatcac

SEQ ID NO: 158 - DNA, 19 bp, synthetic construct
  source 1..19: mol_type = other DNA
  misc_feature 1..19: note = GHH 17
caattacggggtcattagt

SEQ ID NO: 159 - DNA, 21 bp, synthetic construct
  source 1..21: mol_type = other DNA
  misc_feature 1..21: note = GHH 18
ggaggtctatataagcagagc

SEQ ID NO: 160 - DNA, 29 bp, synthetic construct
  source 1..29: mol_type = other DNA
  misc_feature 1..29: note = GHH 19
gtcatcgctattaccatgtggatagcggt

SEQ ID NO: 161 - DNA, 33 bp, synthetic construct
  source 1..33: mol_type = other DNA
  misc_feature 1..33: note = GHH 20
tcatagcattattgactagttattaatagtaat

SEQ ID NO: 162 - DNA, 25 bp, synthetic construct
  source 1..25: mol_type = other DNA
  misc_feature 1..25: note = GHH 21
tgaccgcccaacgacccccgcccaa

SEQ ID NO: 163 - DNA, 30 bp, synthetic construct
  source 1..30: mol_type = other DNA
  misc_feature 1..30: note = GHH 22
acataacttacggtaaatggcccgcctggc

SEQ ID NO: 164 - DNA, 32 bp, synthetic construct
  source 1..32: mol_type = other DNA
  misc_feature 1..32: note = GHH 23
ctagagattggtttagtgaaccgtcagatccg

SEQ ID NO: 165 - DNA, 34 bp, synthetic construct
  source 1..34: mol_type = other DNA
  misc_feature 1..34: note = GHH LB 1
tattctggggggtggggtggggcaggacagcaag

SEQ ID NO: 166 - DNA, 33 bp, synthetic construct
  source 1..33: mol_type = other DNA
  misc_feature 1..33: note = GHH LB 2
cgcatcgagctgaagggcatcgacttcaaggag

SEQ ID NO: 167 - DNA, 33 bp, synthetic construct
  source 1..33: mol_type = other DNA
  misc_feature 1..33: note = GHH LB 3
tcaaggacgacggcaactacaagacccgcgccg

SEQ ID NO: 168 - DNA, 27 bp, synthetic construct
  source 1..27: mol_type = other DNA
  misc_feature 1..27: note = GHH LB 4
tgccttccttgaccctggaaggtgcca

SEQ ID NO: 169 - DNA, 35 bp, synthetic construct
  source 1..35: mol_type = other DNA
  misc_feature 1..35: note = GHH LB 5
ggggaggattgggaagacaatagcaggcatgctgg

SEQ ID NO: 170 - DNA, 29 bp, synthetic construct
  source 1..29: mol_type = other DNA
  misc_feature 1..29: note = GHH LB 6
atggtgagcaagggcgaggagctgttcac

SEQ ID NO: 171 - DNA, 32 bp, synthetic construct
  source 1..32: mol_type = other DNA
  misc_feature 1..32: note = GHH LB 7
cggcgtgcagtgcttcagccgctaccccgacc

SEQ ID NO: 172 - DNA, 31 bp, synthetic construct
  source 1..31: mol_type = other DNA
  misc_feature 1..31: note = GHH LB 8
cgacaagcagaagaacggcatcaaggtgaac

SEQ ID NO: 173 - DNA, 30 bp, synthetic construct
  source 1..30: mol_type = other DNA
  misc_feature 1..30: note = GHH LB 9
ctggcccaccctcgtgaccaccctgaccta

SEQ ID NO: 174 - DNA, 30 bp, synthetic construct
  source 1..30: mol_type = other DNA
  misc_feature 1..30: note = GHH LB 10
atcaccatcaccattgagtttaaacccgct

SEQ ID NO: 175 - DNA, 27 bp, synthetic construct
  source 1..27: mol_type = other DNA
  misc_feature 1..27: note = GHH LB 11
aagcgcgatcacatggtcctgctggag

SEQ ID NO: 176 - DNA, 36 bp, synthetic construct
  source 1..36: mol_type = other DNA
  misc_feature 1..36: note = GHH LB 12
acatgaagcagcacgacttcttcaagtccgccatgc

SEQ ID NO: 177 - DNA, 28 bp, synthetic construct
  source 1..28: mol_type = other DNA
  misc_feature 1..28: note = GHH LB 13
ggacgagctgtacaagtgaccggtcatc

SEQ ID NO: 178 - DNA, 29 bp, synthetic construct
  source 1..29: mol_type = other DNA
  misc_feature 1..29: note = GHH LB 14
aacacccccatcggcgacggccccgtgct

SEQ ID NO: 179 - DNA, 28 bp, synthetic construct
  source 1..28: mol_type = other DNA
  misc_feature 1..28: note = GHH LB 15
cgtgcagctcgccgaccactaccagcag

SEQ ID NO: 180 - DNA, 32 bp, synthetic construct
  source 1..32: mol_type = other DNA
  misc_feature 1..32: note = GHH LB 16
ttcaagatccgccacaacatcgaggacggcag

SEQ ID NO: 181 - DNA, 32 bp, synthetic construct
  source 1..32: mol_type = other DNA
  misc_feature 1..32: note = GHH LB 17
aggtgaagttcgagggcgacaccctggtgaac

SEQ ID NO: 182 - DNA, 31 bp, synthetic construct
  source 1..31: mol_type = other DNA
  misc_feature 1..31: note = GHH LB 18
actacaacagccacaacgtctatatcatggc

SEQ ID NO: 183 - DNA, 30 bp, synthetic construct
  source 1..30: mol_type = other DNA
  misc_feature 1..30: note = GHH LB 19
cggaaagaaccagctggggctcgataccgt

SEQ ID NO: 184 - DNA, 32 bp, synthetic construct
  source 1..32: mol_type = other DNA
  misc_feature 1..32: note = GHH LB 20
ttcgtgaccgccgccgggatcactctcggcat

SEQ ID NO: 185 - DNA, 29 bp, synthetic construct
  source 1..29: mol_type = other DNA
  misc_feature 1..29: note = GHH LB 21
ggatgcggtgggctctatggcttctgagg

SEQ ID NO: 186 - DNA, 31 bp, synthetic construct
  source 1..31: mol_type = other DNA
  misc_feature 1..31: note = GHH LB 22
ccacctacggcaagctgaccctgaagttcat

SEQ ID NO: 187 - DNA, 36 bp, synthetic construct
  source 1..36: mol_type = other DNA
  misc_feature 1..36: note = GHH LB 23
ctcccactgtcctttcctaataaaatgaggaaattg

SEQ ID NO: 188 - DNA, 28 bp, synthetic construct
  source 1..28: mol_type = other DNA
  misc_feature 1..28: note = GHH LB 24
catcgcattgtctgagtaggtgtcattc

SEQ ID NO: 189 - DNA, 34 bp, synthetic construct
  source 1..34: mol_type = other DNA
  misc_feature 1..34: note = GHH LB 25
gacggcaacatcctggggcacaagctggagtaca

SEQ ID NO: 190 - DNA, 24 bp, synthetic construct
  source 1..24: mol_type = other DNA
  misc_feature 1..24: note = GHH LB 26
tggacggcgacgtaaacggccaca

SEQ ID NO: 191 - DNA, 27 bp, synthetic construct
  source 1..27: mol_type = other DNA
  misc_feature 1..27: note = GHH LB 27
tccgccctgagcaaagaccccaacgag

SEQ ID NO: 192 - DNA, 27 bp, synthetic construct
  source 1..27: mol_type = other DNA
  misc_feature 1..27: note = GHH LB 28
ctgcaccaccggcaagctgcccgtgcc

SEQ ID NO: 193 - DNA, 26 bp, synthetic construct
  source 1..26: mol_type = other DNA
  misc_feature 1..26: note = GHH LB 29
gccatctgttgtttgcccctcccccg

SEQ ID NO: 194 - DNA, 33 bp, synthetic construct
  source 1..33: mol_type = other DNA
  misc_feature 1..33: note = GHH LB 30
ccgaaggctacgtccaggagcgcaccatcttct

SEQ ID NO: 195 - DNA, 33 bp, synthetic construct
  source 1..33: mol_type = other DNA
  misc_feature 1..33: note = GHH LB 31
cgacctctagctagagcttggcgtaatcatggt

SEQ ID NO: 196 - DNA, 31 bp, synthetic construct
  source 1..31: mol_type = other DNA
  misc_feature 1..31: note = GHH LB 32
catagctgtttcctgtgtgaaattgtatata

SEQ ID NO: 197 - DNA, 33 bp, synthetic construct
  source 1..33: mol_type = other DNA
  misc_feature 1..33: note = GHH LB 33
agttcagcgtgtccggcgagggcgagggcgatg

SEQ ID NO: 198 - DNA, 26 bp, synthetic construct
  source 1..26: mol_type = other DNA
  misc_feature 1..26: note = GHH LB 34
cggggtggtgcccatcctggtcgagc

SEQ ID NO: 199 - DNA, 31 bp, synthetic construct
  source 1..31: mol_type = other DNA
  misc_feature 1..31: note = GHH LB 35
gctgcccgacaaccactacctgagcacccag

SEQ ID NO: 200 - DNA, 31 bp, synthetic construct
  source 1..31: mol_type = other DNA
  misc_feature 1..31: note = GHH LB 36
gatcagcctcgactgtgccttctagttgcca

SEQ ID NO: 201 - DNA, 21 bp, synthetic construct
  source 1..21: mol_type = other DNA
  misc_feature 1..21: note = GHH CP 1
gactcacattagtcggcccgc

SEQ ID NO: 202 - DNA, 35 bp, synthetic construct
  source 1..35: mol_type = other DNA
  misc_feature 1..35: note = GHH CP 2
cctggctcattgaccacttggtatgccccagtaca

SEQ ID NO: 203 - DNA, 49 bp, synthetic construct
  source 1..49: mol_type = other DNA
  misc_feature 1..49: note = GHH CP 3
gaccttatgaacacagcagggactttcccccgcattgaacattgacgtc

SEQ ID NO: 204 - DNA, 49 bp, synthetic construct
  source 1..49: mol_type = other DNA
  misc_feature 1..49: note = GHH CP 4
ctggcatcagtacatcaagtgcaatagggactttcgaccgcctggagtt

SEQ ID NO: 205 - DNA, 37 bp, synthetic construct
  source 1..37: mol_type = other DNA
  misc_feature 1..37: note = GHH CP 5
tattgacgtcaatgactgccaagtacgccccgtatgt

SEQ ID NO: 206 - DNA, 35 bp, synthetic construct
  source 1..35: mol_type = other DNA
  misc_feature 1..35: note = GHH CP 6
tctacgtggggattgtttgttttgacgcaaatggg

SEQ ID NO: 207 - DNA, 35 bp, synthetic construct
  source 1..35: mol_type = other DNA
  misc_feature 1..35: note = GHH CP 7
aatgggatccaagtctccacctacttggagtacat

SEQ ID NO: 208 - DNA, 35 bp, synthetic construct
  source 1..35: mol_type = other DNA
  misc_feature 1..35: note = GHH CP 8
ccccgccgtaacgctatcataggtaaatatcgcta

SEQ ID NO: 209 - DNA, 30 bp, synthetic construct
  source 1..30: mol_type = other DNA
  misc_feature 1..30: note = GHH CP 10
tccgccccattggcaccaaaatcgcggttt

SEQ ID NO: 210 - DNA, 24 bp, synthetic construct
  source 1..24: mol_type = other DNA
  misc_feature 1..24: note = GHH CP 13
acataacttacggtaaatggcccg

SEQ ID NO: 211 - DNA, 28 bp, synthetic construct
  source 1..28: mol_type = other DNA
  misc_feature 1..28: note = GHH CP 14
ccgcggcgacattgccatatacaacgac

SEQ ID NO: 212 - DNA, 42 bp, synthetic construct
  source 1..42: mol_type = other DNA
  misc_feature 1..42: note = GHH CP 15
gtaaactgccgtcaatgggtggagtaatcactcatctttacg

SEQ ID NO: 213 - DNA, 23 bp, synthetic construct
  source 1..23: mol_type = other DNA
  misc_feature 1..23: note = GHH CP 16
attaataacggggtgtcaataat

SEQ ID NO: 214 - DNA, 21 bp, synthetic construct
  source 1..21: mol_type = other DNA
  misc_feature 1..21: note = GHH CP 18
tcccatacaatgaccattagt

SEQ ID NO: 215 - DNA, 49 bp, synthetic construct
  source 1..49: mol_type = other DNA
  misc_feature 1..49: note = GHH CP 19
cggtaggcgctggcgtgcctgtacggtgactatagggagagccgccacc

SEQ ID NO: 216 - DNA, 33 bp, synthetic construct
  source 1..33: mol_type = other DNA
  misc_feature 1..3: note = GHH CP 20
ttaccatgtggataaacgggatgtcgtaacaac

SEQ ID NO: 217 - DNA, 21 bp, synthetic construct
  source 1..21: mol_type = other DNA
  misc_feature 1..21: note = GHH CP 21
tcatagcattattgactagtt